(19) United States
(12) Patent Application Publication
(10) Pub. No.: US 2024/0035045 A1
(43) Pub. Date: Feb. 1, 2024

(54) ENGINEERED VIRAL CAPSIDS AND METHODS OF USE

(71) Applicant: Avirmax Biopharma Inc., Hayward, CA (US)

(72) Inventors: Shengjiang LIU, Lafayette, CA (US); Haifeng CHEN, Hayward, CA (US); Xiaoming GONG, Hayward, CA (US)

(21) Appl. No.: 18/315,312

(22) Filed: May 10, 2023

Related U.S. Application Data
(63) Continuation of application No. PCT/US2021/058650, filed on Nov. 9, 2021.
(60) Provisional application No. 63/111,739, filed on Nov. 10, 2020.

(51) Int. Cl. C12N 15/86; C07K 14/005; A61K 48/00

(52) U.S. Cl. CPC C12N 15/86; C07K 14/005; A61K 48/0058; C12N 2750/14122; C12N 2750/14143; C12N 2750/14145; C12N 2750/14171

(57) ABSTRACT

Provided herein are compositions and methods comprising modified adeno-associated virus (AAV) capsids. Also provided are methods of utilizing the provided compositions and methods as ocular therapeutics.

### CROSS REFERENCE

This application is a continuation of International Application No. PCT/US2021/058650 filed Nov. 9, 2021, which claims the benefit of U.S. Provisional Application No. 63/111,739 filed Nov. 10, 2020, each of which is incorporated herein by reference its entirety.

### SEQUENCE LISTING

The instant application contains a Sequence Listing which has been submitted electronically in XML format and is hereby incorporated by reference in its entirety. Said XML copy, created on May 10, 2023, is named 59561-701_601_SL.xml and is 202,448 bytes in size.

### BACKGROUND

Adeno-associated virus (AAV) is a small, single-stranded DNA-containing, non-pathogenic parvovirus with a non-enveloped protein capsid that has gained significant attention as an efficient and safe vector for gene transfer. Recombinant AAV vectors have been or are currently being used in 176 phase I, II, and III clinical trials. AAV serotype 2 (AAV2) vectors have shown clinical efficacy in three human diseases: Leber's congenital amaurosis (LCA), aromatic L-amino acid decarboxylase deficiency (AADC), and choroideremia.

In the past decade, at least 12 additional AAV serotype vectors, some derived from non-human primates, have also become available. AAV1 vectors have been successfully used in gene therapy for lipoprotein lipase deficiency, and AAV8 vectors have shown clinical efficacy in potential gene therapy for hemophilia B. More recently, AAV5 vectors have been reported as being effective in hemophilia A. AAV9 vectors have been successfully used in gene therapy for Pompe disease and showed impressive efficacy in gene therapy for spinal muscular atrophy. The AAV1-LPL vector was approved as a drug designated alipogene tiparvovec and marketed under the trade name Glybera in Europe in 2012. In 2017, an AAV2 vector expressing retinal pigment epithelium-specific 65 kDa protein (RPE65) was approved by the Food and Drug Administration as the drug voretigene neparvovec (Luxturna), in the United States. A number of additional phase I and II clinical trials have been or are currently being pursued with AAV1, AAV2, AAV3, AAV5, AAV6, AAV8, AAV9, and AAV10 vectors for potential gene therapy for a wide variety of human diseases.

Despite these remarkable achievements, it has become increasingly clear that the full potential of this technology will only be realized after AAV vectors have been modified for improved cargo delivery.

### SUMMARY

Provided herein is a modified adeno-associated virus (AAV) capsid that comprises an exogenous polypeptide sequence in a VP domain of the AAV capsid as compared to an otherwise comparable unmodified AAV capsid. In an aspect, the exogenous polypeptide sequence comprises a sequence of formula 1: X0-X1-X2-X1-X3-X1-X1-X4-X5 (SEQ ID NO: 93), wherein X0 is Valine (V), Isoleucine (I), Leucine (L), Phenylalanine (F), Tryptophan (W), Tyrosine (Y) or Methionine (M), wherein X1 is Alanine (A), Asparagine (N), Glutamine (Q), Serine (S), Threonine (T), Glutamic Acid (E), Aspartic Acid (D), Lysine (K), Arginine (R), or Histidine (H), wherein X2 is V, I, L, or M, wherein X3 is E, S, or Q, and wherein X4 is K, R, E, or A, optionally wherein formula 1 further comprises X5 being Proline (P) or R. In an embodiment, formula 1 comprises the X5, and wherein the X5 is the Proline (P) or R. In an embodiment, the capsid is of serotype AAV1, AAV2, AAV3, AAV4, AAV5, AAV6, AAV7, AAV8, AAV9, AAV10, AAV11, AAV12, AAV13, and any combination thereof. In an embodiment, the capsid comprises AAV2. In an embodiment, the capsid comprises at least two serotypes. In an embodiment, the at least two serotypes are selected from AAV1, AAV2, AAV3, AAV4, AAV5, AAV6, AAV8, AAV9, AAV11, AAV12, and AAV13. In an embodiment, formula 1 comprises: L-A-L-G-X3-X1-X1-X4 (SEQ ID NO: 94), L-K-L-G-X3-X1-X1-X4 (SEQ ID NO: 95), or V-K-L-G-X3-X1-X1-X4 (SEQ ID NO: 96). In an embodiment, formula 1 comprises the V-K-L-G-X3-X1-X1-X4 (SEQ ID NO: 96), and wherein the V-K-L-G-X3-X1-X1-X4 (SEQ ID NO: 96) comprises: V-K-L-G-X3-X1-T-X4 (SEQ ID NO: 97) or V-K-L-G-X3-X1-X1-K (SEQ ID NO: 98). In an embodiment, formula 1 comprises the L-A-L-G-X3-X1-X1-X4 (SEQ ID NO: 94), and wherein the L-A-L-G-X3-X1-X1-X4 (SEQ ID NO: 94) comprises: L-A-L-G-X3-X1-T-X4 (SEQ ID NO: 99) or L-A-L-G-X3-X1-S-X4 (SEQ ID NO: 100). In an embodiment the L-A-L-G-X3-X1-T-X4 (SEQ ID NO: 99) comprises: a) L-A-L-G-X3-X1-T-R (SEQ ID NO: 101); b) L-A-L-G-X3-X1-T-K (SEQ ID NO: 102); c) L-A-L-G-X3-X1-T-E (SEQ ID NO: 103); or d) L-A-L-G-X3-X1-T-A (SEQ ID NO: 104). In an embodiment, the L-A-L-G-X3-X1-S-X4 (SEQ ID NO: 100) comprises L-A-L-G-X3-X1-S-K (SEQ ID NO: 105). In an embodiment, formula 1 comprises the L-K-L-G-X3-X1-X1-X4 (SEQ ID NO: 95), and wherein the L-K-L-G-X3-X1-X1-X4 (SEQ ID NO: 95) comprises L-K-L-G-X3-X1-T-X4 (SEQ ID NO: 106). In an embodiment, the L-K-L-G-X3-X1-T-X4 (SEQ ID NO: 106) comprises L-K-L-G-X3-X1-T-K (SEQ ID NO: 107). In an embodiment, formula 1 comprises a polypeptide sequence having at least 60%, 70%, 80%, 90%, 95%, 98%, or 99% identity with a sequence of Table 2. In an embodiment, formula 1 comprises a polypeptide sequence of Table 2. In an embodiment, the VP domain of the AAV capsid is VP1. In an embodiment, the VP domain of the AAV capsid is VP2. In an embodiment, the VP domain of the AAV capsid is VP3. In an embodiment, the AAV capsid further comprises a mutation. In an embodiment, the mutation is in a VP1 or VP2 region. In an embodiment, the mutation is in a VP1 or VP3 region. In an embodiment, the mutation is in a VP2 or VP3 region. In an embodiment, the mutation is in VP1, VP2, and VP3 regions. In an embodiment, the mutation is a point mutation, missense mutation, nonsense mutation, deletion, duplication, frameshift, or repeat expansion. In an embodiment, the mutation is a point mutation. In an embodiment, the point mutation comprises a conservative mutation. In an embodiment, the conservative mutation is selected from the group consisting of: a nonpolar aliphatic amino acid to a nonpolar aliphatic amino acid, a polar amino acid to a polar amino acid, a positively charged amino acid to a positively charged amino acid, a negatively charged amino acid to a negatively charged amino acid, and an aromatic amino acid to an aromatic amino acid. In an embodiment, the point mutation comprises a change from a charged amino acid residue to a polar or non-polar amino acid residue. In an embodiment, the charged amino acid is positively charged. In an embodiment, the charged amino acid is negatively charged. In an embodiment, the mutation is in a residue of SEQ ID NO: 1. In an embodiment, the mutation is in a residue selected from the group consisting of: 452, 453, 466, 467, 468, 471, 585, 586, 587, and 588 of SEQ ID NO: 1. In an embodiment, the point mutation is R to A at position 585 or 588 of SEQ ID NO: 1. In an embodiment, the AAV capsid further comprises at least a second exogenous polypeptide sequence in the VP1 domain of the AAV capsid. In an embodiment, the exogenous polypeptide sequence and the second exogenous polypeptide sequence are each independently in a loop of the AAV capsid. In an embodiment, the exogenous polypeptide sequence and the second exogenous polypeptide sequence are each independently in loop 3 and/or loop 4 of the VP1 domain of the AAV capsid.

Provided herein is an adeno-associated virus (AAV) vector that comprises: (a) a modified capsid that comprises an exogenous sequence in at least two loops of a VP domain as compared to an otherwise comparable AAV capsid sequence that lacks the exogenous sequence; and (b) a transgene, wherein said vector, upon contacting with a plurality of cells, has at least 3 fold increased expression of the transgene post the contacting in said plurality of cells as compared to contacting the plurality of cells with an otherwise comparable AAV vector that lacks (a). In an embodiment, the exogenous sequence encodes for a polypeptide that comprises formula 1: X0-X1-X2-X1-X3-X1-X1-X4 (SEQ ID NO: 108), wherein X0 is Valine (V), Isoleucine (I), Leucine (L), Phenylalanine (F), Tryptophan (W), Tyrosine (Y) or Methionine (M), wherein X1 is Alanine (A), Asparagine (N), Glutamine (Q), Serine (S), Threonine (T), Glutamic Acid (E), Aspartic Acid (D), Lysine (K), Arginine (R), or Histidine (H), wherein X2 is V, I, L, or M, wherein X3 is E, S, or Q, and wherein X4 is K, R, E, or A, optionally wherein formula 1 further comprises X5 being Proline (P) or R. In an aspect, formula 1 comprises the X₅, and wherein the X₅ is Proline (P) or R. In an embodiment, the capsid is of serotype AAV1, AAV2, AAV3, AAV4, AAV5, AAV6, AAV7, AAV8, AAV9, AAV10, AAV11, AAV12, AAV13, and any combination thereof. In an embodiment, the capsid comprises AAV2. In an aspect, the capsid comprises at least two serotypes. In an embodiment, the at least two serotypes are AAV2 and AAV5, AAV2 and AAV6, AAV2 and AAV8, AAV2 and AAV9, AAV2 and AAV1, and AAV2 and AAV12. In an embodiment, formula 1 comprises: L-A-L-G-X3-X1-X1-X4 (SEQ ID NO: 94), L-K-L-G-X3-X1-X1-X4 (SEQ ID NO: 95), or V-K-L-G-X3-X1-X1-X4 (SEQ ID NO: 96). In an embodiment, formula 1 comprises the V-K-L-G-X3-X1-X1-X4 (SEQ ID NO: 96), and wherein the V-K-L-G-X3-X1-X1-X4 (SEQ ID NO: 96) comprises: V-K-L-G-X3-X1-T-X4 (SEQ ID NO: 97) or V-K-L-G-X3-X1-X1-K (SEQ ID NO: 98). In an embodiment, formula 1 comprises the L-A-L-G-X3-X1-X1-X4 (SEQ ID NO: 94), and wherein the L-A-L-G-X3-X1-X1-X4 (SEQ ID NO: 94) comprises: L-A-L-G-X3-X1-T-X4 (SEQ ID NO: 99) or L-A-L-G-X3-X1-S-X4 (SEQ ID NO: 100). In an embodiment, the L-A-L-G-X3-X1-T-X4 (SEQ ID NO: 99) comprises: a) L-A-L-G-X3-X1-T-R (SEQ ID NO: 101); b) L-A-L-G-X3-X1-T-K (SEQ ID NO: 102); c) L-A-L-G-X3-X1-T-E (SEQ ID NO: 103); or d) L-A-L-G-X3-X1-T-A (SEQ ID NO: 104). In an embodiment, the L-A-L-G-X3-X1-S-X4 (SEQ ID NO: 100) comprises L-A-L-G-X3-X1-S-K (SEQ ID NO: 105). In an embodiment, formula 1 comprises the L-K-L-G-X3-X1-X1-X4 (SEQ ID NO: 95), and wherein the L-K-L-G-X3-X1-X1-X4 (SEQ ID NO: 95) comprises L-K-L-G-X3-X1-T-X4 (SEQ ID NO: 106). In an embodiment, the L-K-L-G-X3-X1-T-X4 (SEQ ID NO: 106) comprises L-K-L-G-X3-X1-T-K (SEQ ID NO: 107). In an embodiment, formula 1 comprises a polypeptide sequence having at least 60%, 70%, 80%, 90%, 95%, 98%, or 99% identity with a sequence of Table 2. In an embodiment, formula 1 comprises a polypeptide sequence of Table 2. In an embodiment, the VP is VP1. In an embodiment, the VP is VP2. In an embodiment, the VP is VP3. In an embodiment, the at least two loops are loop 3 and loop 4 of the VP1 domain. In an embodiment, the transgene encodes for an ocular therapeutic. In an embodiment, the ocular therapeutic is effective to: reduce at least a symptom of a retinal disease, treat a retinal disease, or eliminate a retinal disease. In an embodiment, the ocular therapeutic is selected from the group consisting of: an antibody or biologically active fragment thereof and a biologic. In an embodiment, the therapeutic is the biologic, and wherein the biologic comprises a polypeptide selected from: Lipoprotein Lipase, Retinoid Isomerohydrolase RPE65, or complement H. In an embodiment, the therapeutic is the antibody or biologically active fragment thereof, and wherein the antibody or biologically active fragment thereof is selected from: anti-VEGF, anti-VEGFL, anti-thrombospondin-1, anti-CD47, anti-TNF-alpha, anti-CD20, anti-CD52, and anti-CD11a, anti-complement 5, and anti-complement 3. In an embodiment, the retinal disease is selected from the group consisting of: Achromatopsia, neovascularization related retinal disorder such as Age-related macular degeneration (AMD), wet-Age-related macular degeneration (wAMD), Geographic atrophy (GA), Diabetic retinopathy (DR), Diabetic macular edema (DME), Glaucoma, Bardet-Biedl Syndrome, Best Disease, Choroideremia, Leber Congenital Amaurosis, Leber Hereditary Optic Neuropathy (LHON), Macular degeneration, Polypoidal choroidal vasculopathy (PCV), Retinitis pigmentosa, Refsum disease, Stargardt disease, Usher syndrome, X-linked retinoschisis (XLRS), Inherited Retinal Disease (IRD), Rod-cone dystrophy, Cone-rod dystrophy, Oguchi disease, Malattia Leventinese (Familial Dominant Drusen), Blue-cone monochromacy, retina vein occlusion (RVO), and Uveitic Macular Oedema (UMO). In an embodiment, the retinal disease is AMD. In an embodiment, the AMD is wet AMD. In an embodiment, the AMD is dry AMD. In an embodiment, the vector further comprises a sequence encoding Rep. In an embodiment, the Rep is modified, and wherein the modification is in at least one of Rep 78, Rep 68, Rep 52 or Rep 40. In an embodiment, the Rep is of a different AAV serotype than the capsid. In an embodiment, the VP is VP1. In an embodiment, the VP is VP2. In an embodiment, the VP is VP3. In an embodiment, the increased expression comprises at least a 5-fold, 10-fold, 20-fold, 50-fold, 100-fold, 200-fold, or 500-fold increase as compared to the contacting the plurality of cells with the otherwise comparable AAV vector that lacks (a). In an embodiment, the modified capsid comprises at least 60%, 70%, 80%, 85%, 90%, 95%, 96%, 97%, 98%, or 99% identity to a sequence that comprises SEQ ID NO: 28-SEQ ID NO:47. In an embodiment, the vector comprises the modified capsid of SEQ ID NO: 34.

Provided herein is an engineered adeno-associated virus (AAV) virion that comprises a modified adeno-associated virus (AAV) capsid.

Provided herein is also a composition that comprises a plurality of AAV virions.

Provided herein is also an engineered cell generated by transfecting a cell with a vector, or an engineered virion.

Provided herein is a plurality of adeno-associated viral (AAV) particles isolated from an engineered cell.

Provided herein is a composition that comprises adeno-associated viral particles in unit dosage form. In an embodiment, a composition is cryopreserved.

Provided is a container that comprises: a) a modified adeno-associated virus (AAV) capsid; b) a vector; or c) an engineered virion. In an embodiment, the container is a vial, syringe, or needle. In an embodiment, the container is configured for ocular delivery.

Provided is a pharmaceutical composition that comprises a) a modified adeno-associated virus (AAV) capsid; b) a vector; or c) an engineered virion. In an embodiment, the pharmaceutical composition is in unit dose form.

Provided is also a method of making engineered cells comprising contacting a plurality of cells with a vector or an engineered virion.

Provided is a modified adeno-associated virus (AAV) capsid that comprises an exogenous sequence from Table 2. In an aspect, the capsid is of a serotype selected from the group consisting of: AAV1, AAV2, AAV5, AAV8, AAV9, and combinations thereof.

Provided is a method of making a modified adeno-associated virus (AAV) capsid, the method comprising introducing a polynucleic acid that encodes a sequence of Table 2 into a sequence encoding an AAV capsid, thereby generating a modified AAV capsid. In an aspect, the AAV capsid is of a serotype selected from the group consisting of: AAV1, AAV2, AAV5, AAV8, AAV9, and combinations thereof.

Provided is a method for treating a disease or condition in a subject in need thereof, the method comprising administering a therapeutically effective amount of a pharmaceutical composition that comprises an adeno-associated virus (AAV) vector that comprises a modified capsid that comprises an exogenous polypeptide sequence in at least two loops of a VP domain as compared to an otherwise comparable AAV capsid sequence that lacks the exogenous polypeptide sequence, wherein the exogenous polypeptide sequence comprises a sequence of Table 2. In an aspect, the AAV vector further comprises a sequence that comprises a transgene.

Provided is a method for treating a disease or condition in a subject in need thereof, the method comprising administering a therapeutically effective amount of a pharmaceutical composition that comprises an adeno-associated virus (AAV) vector that comprises: (a) a modified capsid that comprises an exogenous sequence in at least two loops of a VP domain as compared to an otherwise comparable AAV capsid sequence that lacks the exogenous sequence; and (b) a transgene, wherein the vector, upon contacting with a plurality of cells, has at least 3 fold increased expression of the transgene post transfection in the plurality of cells as compared to contacting the plurality of cells with an otherwise comparable AAV vector that lacks (a). In an aspect, the increased expression comprises at least a 5-fold, 10-fold, 20-fold, 50-fold, 100-fold, 200-fold, or 500-fold increase as compared to the contacting the plurality of cells with the otherwise comparable AAV vector that lacks (a).

Provided is also a method for treating a disease or condition in a subject in need thereof, the method comprising administering a therapeutically effective amount of a pharmaceutical composition that comprises an adeno-associated virus (AAV) vector that comprises a modified capsid that comprises an exogenous polypeptide sequence in a VP domain of the AAV capsid as compared to an otherwise comparable unmodified AAV capsid, the exogenous polypeptide sequence comprising a sequence of formula 1: X0-X1-X2-X1-X3-X1-X1-X4 (SEQ ID NO: 108), wherein X0 is Valine (V), Isoleucine (I), Leucine (L), Phenylalanine (F), Tryptophan (W), Tyrosine (Y) or Methionine (M), wherein X1 is Alanine (A), Asparagine (N), Glutamine (Q), Serine (S), Threonine (T), Glutamic Acid (E), Aspartic Acid (D), Lysine (K), Arginine (R), or Histidine (H), wherein X2 is V, I, L, or M, wherein X3 is E, S, or Q, and wherein X4 is K, R, E, or A, optionally wherein formula 1 further comprises X5 being Proline (P) or R. In an aspect, formula 1 comprises the X5, and wherein the X5 is Proline (P) or R. In an aspect, the capsid is of serotype AAV1, AAV2, AAV3, AAV4, AAV5, AAV6, AAV7, AAV8, AAV9, AAV10, AAV11, AAV12, AAV13, and any combination thereof. In an aspect, the capsid comprises AAV2. In an embodiment, the capsid comprises at least two serotypes. In an embodiment, the at least two serotypes are selected from AAV1, AAV2, AAV3, AAV4, AAV5, AAV6, AAV8, AAV9, AAV11, AAV12, and AAV13. In an embodiment, formula 1 comprises: L-A-L-G-X3-X1-X1-X4 (SEQ ID NO: 94), L-K-L-G-X3-X1-X1-X4 (SEQ ID NO: 95), or V-K-L-G-X3-X1-X1-X4 (SEQ ID NO: 96). In an embodiment, formula 1 comprises the V-K-L-G-X3-X1-X1-X4 (SEQ ID NO: 96), and wherein the V-K-L-G-X3-X1-X1-X4 (SEQ ID NO: 96) comprises: V-K-L-G-X3-X1-T-X4 (SEQ ID NO: 97) or V-K-L-G-X3-X1-X1-K (SEQ ID NO: 98). In an embodiment, formula 1 comprises the L-A-L-G-X3-X1-X1-X4 (SEQ ID NO: 94), and wherein the L-A-L-G-X3-X1-X1-X4 (SEQ ID NO: 94) comprises: L-A-L-G-X3-X1-T-X4 (SEQ ID NO: 99) or L-A-L-G-X3-X1-S-X4 (SEQ ID NO: 100). In an embodiment, the L-A-L-G-X3-X1-T-X4 (SEQ ID NO: 99) comprises: a) L-A-L-G-X3-X1-T-R (SEQ ID NO: 101); b) L-A-L-G-X3-X1-T-K (SEQ ID NO: 102); c) L-A-L-G-X3-X1-T-E (SEQ ID NO: 103); or d) L-A-L-G-X3-X1-T-A (SEQ ID NO: 104). In an embodiment, the L-A-L-G-X3-X1-S-X4 (SEQ ID NO: 100) comprises L-A-L-G-X3-X1-S-K (SEQ ID NO: 105). In an embodiment, formula 1 comprises the L-K-L-G-X3-X1-X1-X4 (SEQ ID NO: 95), and wherein the L-K-L-G-X3-X1-X1-X4 (SEQ ID NO: 95) comprises L-K-L-G-X3-X1-T-X4 (SEQ ID NO: 106). In an embodiment, the L-K-L-G-X3-X1-T-X4 (SEQ ID NO: 106) comprises L-K-L-G-X3-X1-T-K (SEQ ID NO: 107). In an embodiment, formula 1 comprises a polypeptide sequence having at least 60%, 70%, 80%, 90%, 95%, 98%, or 99% identity with a sequence of Table 2. In an embodiment, formula 1 comprises a polypeptide sequence of Table 2. In an embodiment, the administering is via intravitreal injection, subretinal injection, microinjection, or super ocular injection. In an embodiment, the administering is via intravitreal injection. In an embodiment, the disease or condition is ocular. In an embodiment, the disease or condition is non-ocular. In an embodiment, the ocular disease or condition is of the retina. In an embodiment, the ocular disease or condition is selected from the group consisting of: Achromatopsia, neovascularization related retinal disorder such as Age-related macular degeneration (AMD), wet-Age-related macular degeneration (wAMD), Geographic atrophy (GA), Diabetic retinopathy (DR), Diabetic macular edema (DME), Glaucoma, Bardet-Biedl Syndrome, Best Disease, Choroideremia, Leber Congenital Amaurosis, Leber Hereditary Optic Neuropathy (LHON), Macular degeneration, Polypoidal choroidal vasculopathy (PCV), Retinitis pigmentosa, Refsum disease, Stargardt disease, Usher syndrome, X-linked retinoschisis (XLRS), Inherited Retinal Disease (IRD), Rod-cone dystrophy, Cone-rod dystrophy, Oguchi disease, Malattia Leventinese (Familial Dominant Drusen), Blue-cone monochromacy, retina vein occlusion (RVO), and Uveitic Macular Oedema (UMO). In an embodiment, the ocular disease or condition is AMD. In an embodiment, the AMD is wet AMD. In an embodiment, the AMD is dry AMD. In an embodiment, the administering is sufficient to reduce at least a symptom of the disease or condition, treat the disease or condition, and/or eliminate the disease or condition. In an embodiment, the vector further comprises a transgene that codes for a therapeutic polypeptide. In an embodiment, the therapeutic is selected from the group consisting of: an antibody or biologically active fragment thereof or a biologic. In an embodiment, the therapeutic is the biologic, and wherein the biologic comprises a polypeptide selected from: Lipoprotein Lipase, Retinoid Isomerohydrolase RPE65, complement H. In an embodiment, the therapeutic is the antibody or biologically active fragment thereof, and wherein the antibody or biologically active fragment thereof is selected from: anti-VEGF, anti-VEGFL, anti-thrombospondin-1, anti-CD47, anti-TNF-alpha, anti-CD20, anti-CD52, and anti-CD11a. In an embodiment, prior to the administering the subject undergoes genetic testing. In an embodiment, the genetic testing detects a mutation in a gene selected from: RPE65, CRB1, AIPL1, CFH, or RPGRIP. In an embodiment, the administering comprises delivering a dosage of the vector of about 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, or 1.0×10¹⁵ vg. In an embodiment, the administering is repeated. In an embodiment, the administering is performed: twice daily, every other day, twice a week, bimonthly, trimonthly, once a month, every other month, semiannually, annually, or biannually. In an embodiment, the method further comprises administering a secondary therapy. In an embodiment, the VP comprises VP1. In an embodiment, the VP comprises VP2. In an embodiment, the VP comprises VP3.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows structural modeling of an AAV2 capsid and its variants. Left panel shows a 60-mer of AAV2 capsid colored gray with reference monomer colored dark gray and a different color for the insertion on each monomer. Right panel shows a monomer VP of AAV2 with the peptide insertion at loop3 and loop4.

FIG. 2 shows SDS-PAGE showing the purity of AAV2 vectors. M, protein ladders; lanes 1 to 10, AMI051-AMI057, V467, 7m8 control vector, and Wildtype (WT) control AAV2. Each lane was loaded with 1e+11 vg.

FIG. 3A shows transduction efficiency of modified AAV2 capsid variants in human cell lines. HEK293 and ARPE-19 cells were transduced with the wild-type (wt., AAV2-V104) and modified capsid variants (AAV2-V466, AAV2-V467, and AAV2-V471) of AAV2-CMV-GFP vectors at a MOI of 1e+5 vg/cell. FIG. 3B shows transduction efficiency of modified AAV2 capsid variants in human cell lines. HEK293 transgene expression was analyzed by fluorescence microscopy 72 hours post-transduction. FIG. 3C and FIG. 3D show ELISA measurement of VEGF-Trap expression in human HEK293 cells (FIG. 3C) and in human ARPE-19 cells (FIG. 3D) transduced by the modified AAV described herein: modified AAV1 comprising insertion of LALGQTTKPA exogenous polypeptide (SEQ ID NO: 14) after amino acid residue position 588 (asparagine, N); modified AAV2 comprising S588 insertion of LALGQTTKPA exogenous polypeptide (SEQ ID NO: 14) after amino acid residue position 588 (serine, S); and modified AAV6 comprising modified AAV2 comprising S588 insertion of LALGQTTKPA exogenous polypeptide (SEQ ID NO: 14) after amino acid residue position 588 (serine, S).

FIG. 4 are representative flat mount images of GFP expression on Day 28. GFP expression was examined on mouse retinal flat mounts following wholemount staining. GFP expression in Groups 2 and 3 was significantly more robust, thus images were acquired at a lower gain setting so that subcellular GFP could be discerned.

FIG. 5 are representative images from the immunohistochemical analysis of Day 28 eyes. Three different antibody cocktails were utilized to evaluate the expressed GFP localization. The GFP was so abundant in Group 3 eyes that the primary and secondary antibodies did not fully capture all of it, resulting in detection of endogenous GFP in addition to the immunolabeled GFP (Group 3, column 1).

FIG. 6 is a graphical representation of GFP expression across all three antibody cocktails and eyes imaged. GFP expression was scored across all retinal and extra-retinal layers and across all antibody cocktails used, for a survey of ocular expression across all eyes; each eye is represented by a single row. Gray cells indicate where positive GFP expression was observed; white cells indicate absent GFP expression. Abbreviations: GCL/ILM—ganglion cell layer/Inner limiting membrane; IPL—inner plexiform layer; INL—inner nuclear layer; OPL—outer plexiform layer; ONL—outer nuclear layer; IS—inner segment; OS—outer segment; RPE—retinal pigmented epithelium; C—choroid; S—sclera; M—muscle; ONH—optic nerve head.

FIG. 7 illustrates in vivo fundus imaging and immunohistochemical (IHC) staining of the eye, where wild type (wt) AAV2 was administered intravitreally (IVT) to the eye of a mouse. The Wt AAV2-GFP did not lead to enough GFP expression as shown both the fundus imaging and IHC section. Figure discloses SEQ ID NO: 133.

FIG. 8 illustrates in vivo fundus imaging and IHC staining of the eye, where the modified AAV2 described herein (AAV2-AMI053-GFP comprising modified AAV2 with LKLGQTTKPA (SEQ ID NO: 13) inserted after amino acid residue 587) was administered via IVT route to the eye of a mouse. The modified AAV2 exhibited GFP expression in fundus imaging and IHC sections with GFP signal detected in multiple layers of cells. Figure discloses SEQ ID NO: 116.

FIG. 9 illustrates in vivo fundus imaging and IHC staining of the eye, where the modified AAV2 described herein (AAV2-AMI054-GFP comprising modified AAV2 with LALGQTTKPA (SEQ ID NO: 14) inserted after amino acid residue 587) was administered via IVT route to the eye of a mouse. The modified AAV2 exhibited GFP expression in fundus imaging and IHC sections with GFP signal detected in multiple layers of cells. Figure discloses SEQ ID NO: 117.

FIG. 10 illustrates in vivo fundus imaging and IHC staining of the eye, where the modified AAV2 described herein (AAV2-V466-GFP comprising modified AAV2 with LALGETTRPA (SEQ ID NO: 6) inserted after amino acid residue 453) was administered via IVT route to the eye of a mouse. The modified AAV2 exhibited GFP expression in fundus imaging and IHC sections with GFP signal detected in multiple layers of cells. Figure discloses SEQ ID NO: 110.

FIG. 11 illustrates AAV retina transduction index (ARCTI) illustrating AAV transduction in the different optic fiber layers of the eye transduced with either the wild type (Wt) or the modified AAV described herein.

FIG. 12 illustrates results mouse laser-induced choroidal neovascularization (LCNV) study, where modified AAV was administered to the mouse prior to the LCNV. AAV1, AAV2, or AAV6 was modified with insertion of LALGQTTKPA (SEQ ID NO: 14) in the VP1 capsid and carried Aflibercept (VEGF-Trap) as the transgene for delivery to the eye of the mouse. Modified AAV1 (Group 5, AAV1. N54-Aflibercept) comprises LALGQTTKPA (SEQ ID NO: 14) inserted after S588 amino acid residue of the VP1 capsid. AAV2 (Group 3, AAV2. N54-Aflibercept) comprises LALGQTTKPA (SEQ ID NO: 14) inserted after N587 amino acid residue of the VP1 capsid. AAV6 (Group 4, AAV6. N54-Aflibercept) comprises LALGQTTKPA (SEQ ID NO: 14) inserted after S588 amino acid residue of the VP1 capsid.

FIG. 13A illustrates area of choroidal neovascularization in of the eye from LCNV study of FIG. 12. ΔAflibercept was a sham control, where AAV comprising disruption in open reading frame was administered to the eye of the mouse. Compared to the vehicle group, all three modified AAV groups showed significant decrease in area of choroidal neovascularization in the eye (AAV6.N54-Aflibercept, p<0.001; AAV1.N54-Aflibercept, p<0.01; and AAV2.N54-Aflibercept, p<0.05). FIG. 13B illustrates that the group administered with AAV6.N54-Aflibercept continued to show significant decrease in in area of choroidal neovascularization in the eye (AAV6.N54-Aflibercept, p<0.05).

FIG. 14 illustrates aflibercept concentration in ocular (left and middle panels) and serum (right panel) samples obtained from the LCNV study mouse from FIG. 12.

FIG. 15 illustrates fluorescein angiography (FA) analysis of retina Aflibercept protective effect against retina damage induced by LCNV in the mouse from FIG. 12. Correlation of Aflibercept concentration in eye cup versus retina injury caused by lasering was inversely correlated.

FIG. 16 illustrates Day 21 Fundus images. The posterior section of the eye was imaged in both color and cobalt blue channels. Imaging acquisition settings for GFP expression were calibrated on Group 6 animals and levels were kept consistent across the study.

FIG. 17 illustrates representative images from the immunohistochemical analysis of day 28 eyes. Cryosections (14 μm) were evaluated for the localization of GFP, RPE65, phalloidin and DAPI. Merged images are shown on the left and pullouts of the GFP channel are shown on the right.

FIG. 18 illustrates representative images from the Immunohistochemical Analysis of Day 28 Eyes Using Confocal Microscopy. Cryosections (14 μm) were evaluated for the localization of GFP, RPE65, phalloidin, and DAPI. Merged images are shown on the left and the GFP are shown on the right.

FIG. 19A illustrates intravitreous delivery of AAV2.AMI054-GFP (modified AAV2 comprising the LALGQTTKPA (SEQ ID NO: 14) insertion) in to eye of a pig. IHC staining showed GFP fluorescence in retina layers. FIG. 19B illustrates GFP expression in each retinal layer of the eye administered with AAV2.AMI054-GFP.

FIG. 20 illustrates a comparison of GFP expression in pig retina between eye administered with AAV2.AMI054-GFP or with AAV2.7M8-GFP (control). The retina transduced with AAV2.AMI054-GFP showed GFP expression in all retinal layers as opposed to the retina transduced with AAV2.7M8-GFP. The GFP fluorescent in the AAV2.AMI054-GFP transduced retina was twice as intense as the AAV2.7M8-GFP transduced retina.

### DETAILED DESCRIPTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

### Definitions

The terms “AAV,” “AAV construct,” or “recombinant AAV” or “AAV” refer to adeno-associated virus of any of the known serotypes, including AAV-1, AAV-2, AAV-3, AAV-4, AAV-5, AAV-6, AAV-7, AAV-8, AAV-9, AAV-10, AAV-11, AAV-12, AAV-13, or scAAV, rh10, chimeric or hybrid AAV, or any combination, derivative, or variant thereof. AAV is a small non-enveloped single-stranded DNA virus. They are non-pathogenic parvoviruses and can require helper viruses, such as adenovirus, herpes simplex virus, vaccinia virus, and CMV, for replication. Wild-type AAV is common in the general population, and is not associated with any known pathologies. A hybrid AAV is an AAV comprising a capsid protein of one AAV serotype and genomic material from another AAV serotype. A chimeric AAV comprises genetic and/or protein sequences derived from two or more AAV serotypes, and can include mutations made to the genetic sequences of those two or more AAV serotypes. An exemplary chimeric AAV can comprise a chimeric AAV capsid, for example, a capsid protein with one or more regions of amino acids derived from two or more AAV serotypes. An AAV variant is an AAV comprising one or more amino acid mutations in its genome or proteins as compared to its parental AAV, e.g., one or more amino acid mutations in its capsid protein as compared to its parental AAV. AAV, as used herein, includes avian AAV, bovine AAV, canine AAV, equine AAV, primate AAV, non-primate AAV, and ovine AAV, wherein primate AAV refers to AAV that infect non-primates, and wherein non-primate AAV refers to AAV that infect non-primate animals, such as avian AAV that infects avian animals. In some cases, the wild-type AAV contains rep and cap genes, wherein the rep gene is required for viral replication and the cap gene is required for the synthesis of capsid proteins. As used herein, the terms “recombinant AAV” and “rAAV” are interchangeable.

The terms “recombinant AAV vector” or “AAV vector” or “AAV vector” refer to a vector derived from any of the AAV serotypes mentioned above. In some cases, an AAV vector can comprise one or more of the AAV wild-type genes deleted in whole or part, such as the rep and/or cap genes, but contains functional elements that are required for packaging and use of AAV virus for gene therapy. For example, functional inverted terminal repeats or ITR sequences that flank an open reading frame or exogenous sequences cloned in are known to be important for replication and packaging of an AAV virion, but the ITR sequences can be modified from the wild-type nucleotide sequences, including insertions, deletions, or substitutions of nucleotides, so that the AAV is suitable for use for the embodiments described herein, such as a gene therapy or gene delivery system. In some aspects, a self-complementary vector (sc) can be used, such as a self-complementary AAV vector, which can bypass the requirement for viral second-strand DNA synthesis and can lead to higher rate of expression of a transgene protein, as described in Wu, Hum Gene Ther. 2007, 18(2):171-82, incorporated by reference herein. In some aspects, AAV vectors can be generated to allow selection of an optimal serotype, promoter, and transgene. In some cases, the vector can be targeted vector or a modified vector that selectively binds or infects immune cells.

The terms “AAV virion” or “AAV virion” refer to a virus particle comprising a capsid comprising at least one AAV capsid protein that encapsidates an AAV vector as described herein, wherein the vector can further comprise a heterologous polynucleotide sequence or a transgene in some embodiments. A virion can be an engineered virion.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “subject,” “host,” “individual,” and “patient” are as used interchangeably herein to refer to animals, typically mammalian animals. Any suitable mammal can be administered a composition as described herein (such as an engineered guide RNA) or treated by a method as described herein. A subject can be a vertebrate or an invertebrate. A subject can be a laboratory animal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

The term “protein”, “peptide”, and “polypeptide” are used interchangeably and in their broadest sense to refer to a compound of two or more subunit amino acids, amino acid analogs or peptidomimetics. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains which may compromise their respective functions.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

### Overview

Provided herein are modified adeno-associated virus (AAV) capsid-containing compositions and methods of using the same. A modified AAV capsid can comprise exogenous sequences as compared to an otherwise comparable unmodified AAV capsid. Exogenous sequences can refer to exogenous polypeptide sequences. AAV capsids can be modified to confer upon them, and any compositions and/or methods in which they are utilized, improved functionality thereby resulting in better therapeutics, particularly for ocular use.

The AAV wild-type (WT) genome contains at least three genes: rep, cap, and X. The X gene, first described in 1999, is located at the 3′ end of the genome (nucleotides 3929-4393 in AAV2) and seems to code for a protein with supportive function in genome replication. Significantly more information is available for rep and cap. The rep gene is located in the first half of the AAV WT genome and codes for a family of non-structural proteins (Rep proteins) required for viral transcription control and replication as well as packaging of viral genomes into the newly produced, pre-assembled capsids. The second half of the AAV genome contains the cap gene, which codes for the viral proteins (VPs) VP1, VP2, and VP3, and the assembly-activating protein (AAP). Transcription of all VPs, which are the capsid monomers, is controlled by a single promoter (p40 in case of AAV2) resulting in a single mRNA. Splicing (VP1) and an unusual translational start codon (VP2) are responsible for an approximately 10 times lower presence of VP1 and VP2 compared with VP3. As expected, when encoded by a single gene, AAV VPs share most of their amino acids. Specifically, the entire VP3 sequence is also contained within VP2 and VP1 (“common VP3 region”), and also VP2 and VP1 share approximately 65 amino acids (“common VP1/VP2 region”). Only VP1 contains a unique sequence at its N terminus (approximately 138 amino acids, VP1 unique). AAP was identified in 2010 as a 23 kD protein encoded in an alternative cap ORF. It is used for stabilizing and transporting newly produced VP proteins from the cytoplasm into the cell nucleus. Interestingly, while AAV serotypes 1-3, 6-9, and rh10 failed to produce capsids in the absence of AAP, a low but detectable capsid production was reported for AAV4 and AAV5.

In an aspect, an AAV can comprise a modification. A modification can be of a rep, cap, and/or X coding polypeptide sequence of an AAV. In some cases, the modification can be of a cap polypeptide. A cap polypeptide can be modified in any one of the VP domains, for example VP1, VP2, and/or VP3. In some cases, VP1 is modified. In some cases, VP2 is modified. In some cases, VP3 is modified. In some aspects, two or all of the VP domains can be modified. In some cases, VP1 and VP2 are modified. In some cases, VP1 and VP3 are modified. Additionally, VP2 and VP3 can be modified or VP1, VP2, and VP3 are modified. Other combinations are contemplated, such as modifications in Rep and Cap, Cap and X, Rep and X, and/or Rep, Cap, and X. Any combination of domains can be modified such as any one of the aforementioned VP modifications in conjunction with a Rep and/or X modification. In some cases, Rep and VP1 and/or VP2 are modified. In some aspects, a subject Rep is modified. A rep modification can comprise a modification as provided herein and can be in at least one of Rep 78, Rep 68, Rep 52 or Rep 40. In some cases, a Rep is of a different AAV serotype than a subject capsid.

In some cases, a modification is of an AAV capsid. Capsids of AAV serotypes are assembled from 60 VP monomers with approximately 50 copies of VP3, 5 copies of VP2, and 5 copies of VP1. Topological prominent capsid surface structures are pores or “channel-like-structures” at each fivefold, depressions at each twofold, and three protrusions surrounding each threefold axis of symmetry. The pores allow exchange between the capsid interior and the outside. The depressions, more precisely the floor at each twofold axis, are the thinnest part of the viral capsid. The protrusions around the threefold axis harbor five of the nine so-called variable regions (VRs). Specifically, VR-IV, -V, and -VIII form loops (loop 1-4) at the top of the protrusions, while VR-VI and -VII are found at their base. VRs differ between serotypes and are responsible for serotype-specific variations in antibody and receptor binding. Because of their exposed positions and their function in receptor binding, VRs forming loops of the protrusions are ideal positions for capsid modifications aiming to re-direct or expand AAV tropism (cell surface targeting). While a re-directed tropism (vector retargeting) combines ablation of natural receptor binding, for example by site-directed mutagenesis, with insertion of a ligand that mediates transduction through a novel non-natural AAV receptor, AAV vectors with tropism expansion gain the ability to transduce cells through an extra receptor while maintaining their natural receptor binding abilities.

In some aspects, a modification of an AAV capsid, can refer to an insertion of an exogenous polypeptide sequence. In other aspects, a modification can refer to a deletion in a polypeptide sequence. A modification can also refer to a modification of at least one amino acid residue, canonical or non-canonical, in a polypeptide sequence.

An insertion can comprise inserting at least 1 exogenous amino acid residue into a sequence coding an AAV capsid. The amino acid can refer to a canonical amino acid or a non-canonical amino acid. Any number of amino acid residues can be inserted. In some cases, an insertion site can be in the GH loop, or loop IV, of the AAV capsid protein, e.g., in a solvent-accessible portion of the GH loop, or loop IV, of the AAV capsid protein. For the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955.

In some cases, a modification comprises insertion of an exogenous polypeptide sequence that comprises a sequence of Formula 1:

(SEQ ID NO: 108)
X0-X1-X2-X1-X3-X1-X1-X4

In some cases, X0 is Valine (V), Isoleucine (I), Leucine (L), Phenylalanine (F), Tryptophan (W), Tyrosine (Y) or Methionine (M). In some cases, X1 is Alanine (A), Asparagine (N), Glutamine (Q), Serine (S), Threonine (T), Glutamic Acid (E), Aspartic Acid (D), Lysine (K), Arginine (R), or Histidine (H). In some cases, X2 is V, I, L, or M, wherein X3 is E, S, or Q. In some cases, X4 is K, R, E, or A. In some cases, Formula 1 further comprises X5. X5 can be Proline (P) or R.

In some cases, Formula 1 comprises: L-A-L-G-X3-X1-X1-X4 (SEQ ID NO: 94), L-K-L-G-X3-X1-X1-X4 (SEQ ID NO: 95), or V-K-L-G-X3-X1-X1-X4 (SEQ ID NO: 96). In some cases, Formula 1 comprises V-K-L-G-X3-X1-X1-X4 (SEQ ID NO: 96). In some cases, an exogenous polypeptide is V-K-L-G-X3-X1-T-X4 (SEQ ID NO: 97) and/or V-K-L-G-X3-X1-X1-K (SEQ ID NO: 98). In some cases, an exogenous polypeptide comprises L-A-L-G-X3-X1-X1-X4 (SEQ ID NO: 94). In some cases, an exogenous polypeptide comprises L-A-L-G-X3-X1-T-X4 (SEQ ID NO: 99) and/or L-A-L-G-X3-X1-S-X4 (SEQ ID NO: 100). In some cases, an exogenous polypeptide comprises: L-A-L-G-X3-X1-T-R (SEQ ID NO: 101), L-A-L-G-X3-X1-T-K (SEQ ID NO: 102), L-A-L-G-X3-X1-T-E (SEQ ID NO: 103), and/or L-A-L-G-X3-X1-T-A (SEQ ID NO: 104). In some cases, an exogenous polypeptide comprises L-A-L-G-X3-X1-S-K (SEQ ID NO: 105). In some cases, an exogenous polypeptide comprises L-K-L-G-X3-X1-X1-X4 (SEQ ID NO: 95). In some cases, an exogenous polypeptide comprises: L-K-L-G-X3-X1-T-X4 (SEQ ID NO: 106). In some cases, an exogenous polypeptide comprises: L-K-L-G-X3-X1-T-K (SEQ ID NO: 107).

In some cases, an exogenous polypeptide comprises a sequence of Formula 1. In some cases, a sequence of Formula I comprises a polypeptide sequence having at least 60%, 62%, 64%, 66%, 68%, 70%, 72%, 74%, 76%, 78%, 80%, 82%, 84%, 86%, 88%, 90%, 92%, 94%, 96%, 98%, or up to about 100% identity with a sequence of Table 2. In some cases, an exogenous polypeptide is one of Table 2 with 0-2 modifications to a residue. In some cases, an exogenous polypeptide comprises a sequence having at least 90%, 95%, 97%, or up to about 99% sequence identity to SEQ ID NO: 13. In some cases, an exogenous polypeptide comprises SEQ ID NO: 13 or a polypeptide having from 0-2 substitutions to SEQ ID NO: 13.

In some cases, at least 2 of the exogenous polypeptides, such as those described by Formula 1, are inserted into a capsid sequence of an AAV provided herein. The at least 2 exogenous polypeptides can be inserted into the same location or at different locations. In an aspect, any one of the exogenous polypeptide sequences provided in Table 2 can be inserted into an unmodified AAV capsid sequence, such as those wildtype sequences provided in Table 1, to generate a modified AAV capsid.

Similarly, a deletion can comprise deleting at least 1 amino acid residue in a sequence that codes for an AAV capsid. Any number of amino acids can be deleted.

In some cases, at least, or at most: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or up to about 50 exogenous amino acid residues can be inserted and/or deleted in a polypeptide sequence that codes for an AAV capsid. In some cases, at least or at most: 1-5, 5-10, 10-15-20, or combinations thereof of exogenous amino acid residues can be inserted and/or deleted in a polypeptide sequence that codes for an AAV capsid. In some cases, from about or up to about: 5 amino acids to about 11 amino acids are inserted in an insertion site in the GH loop or loop IV of the capsid protein relative to a corresponding unmodified AAV capsid protein. For example, the insertion site can be between amino acids 587 and 588 of AAV2, or the corresponding positions of the capsid subunit of another AAV serotype. It should be noted that the insertion site 587-588 is based on an AAV2 capsid protein. From about 5 amino acids to about 11 amino acids can be inserted in a corresponding site in an AAV serotype other than AAV2 (e.g., AAV5, AAV6, AAV8, AAV9, etc.).

In some embodiments, the insertion site is a single insertion site between two adjacent amino acids located between amino acids 570-614 of VP1 of any AAV serotype, e.g., the insertion site is between two adjacent amino acids located in amino acids 570-610, amino acids 580-600, amino acids 570-575, amino acids 575-580, amino acids 580-585, amino acids 585-590, amino acids 590-600, or amino acids 600-614, of VP1 of any AAV serotype or variant. For example, the insertion site can be between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600.

In some aspects, an insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 588 and 589 of AAV10.

As another example, the insertion site can be between amino acids 450 and 460 of an AAV capsid protein, as shown in Table 1. For example, the insertion site can be at (e.g., immediately N-terminal to) amino acid 453 of AAV2, at amino acid 454 of AAV1, at amino acid 454 of AAV6, at amino acid 456 of AAV7, at amino acid 456 of AAV8, at amino acid 454 of AAV9, or at amino acid 456 of AAV10.

In some embodiments, a subject capsid protein includes a GH loop comprising an amino acid sequence having at least about 85%, at least about 90%, at least about 95%, at least about 98%, at least about 99%, or 100%, amino acid sequence identity to an amino acid sequence set forth in Table 1. Those skilled in the art would know, based on a comparison of the amino acid sequences of capsid proteins of various AAV serotypes, where an insertion site “corresponding to amino acids 587-588 of AAV2” would be in a capsid protein of any given AAV serotype.

In some cases, an exogenous polypeptide can have from 0 to 4 spacer amino acids (Yi-Y4) at the amino terminus and/or at the carboxyl terminus of any one of the exemplary polypeptides of Table 2 or Formula 1. Suitable spacer amino acids include, but are not limited to, leucine, alanine, glycine, and/or serine.

A modification of an AAV capsid can comprise a modification of at least one amino acid residue in a polypeptide sequence. In some cases, a modification can be made at any AAV capsid position, as described herein, and can include any number of modifications. In some cases, a modification can comprise a mutation. A mutation can comprise: a point mutation, missense mutation, nonsense mutation, deletion, duplication, frameshift, and/or repeat expansion.

In an aspect, an amino acid can be a non-polar, aliphatic residue such as glycine, alanine, valine, leucine, isoleucine, or proline. In an aspect, an amino acid residue is aromatic and is phenylalanine, tyrosine, or tryptophan. In an aspect, an amino acid residue is polar, non-charged and is serine, threonine, cysteine, methionine, asparagine, or glutamine. In an aspect, an amino acid is positively charged and is lysine, arginine, or histidine. In an aspect, an amino acid is negatively charged and is aspartate or glutamate.

In some cases, a mutation is a point mutation. A point mutation comprises a change from a charged amino acid residue to a polar or non-polar amino acid residue. In some cases, the charged amino acid is positively charged. In some cases, the charged amino acid is negatively charged.

A point mutation can be a conservative mutation. Non-limiting examples of conservative mutations comprise: a nonpolar aliphatic amino acid to a nonpolar aliphatic amino acid, a polar amino acid to a polar amino acid, a positively charged amino acid to a positively charged amino acid, a negatively charged amino acid to a negatively charged amino acid, and an aromatic amino acid to an aromatic amino acid. For example, 20 naturally occurring amino acids can share similar characteristics. Aliphatic amino acids can be: glycine, alanine, valine, leucine, or isoleucine. Hydroxyl or sulfur/selenium-containing amino acids can be: Serine, cysteine, selenocysteine, threonine, or methionine. A cyclic amino acid can be proline. An aromatic amino acid can be phenylalanine, tyrosine, or tryptophan. A basic amino acid can be histidine, lysine, and arginine. An acidic amino acid can be aspartate, glutamate, asparagine, or glutamine. A conservative mutation can be, serine to glycine, serine to alanine, serine to serine, serine to threonine, serine to proline. A conservative mutation can be arginine to asparagine, arginine to lysine, arginine to glutamine, arginine to arginine, arginine to histidine. A conservative mutation can be Leucine to phenylalanine, leucine to isoleucine, leucine to valine, leucine to leucine, leucine to methionine. A conservative mutation can be proline to glycine, proline to alanine, proline to serine, proline to threonine, proline to proline. A conservative mutation can be threonine to glycine, threonine to alanine, threonine to serine, threonine to threonine, threonine to proline. A conservative mutation can be alanine to glycine, alanine to threonine, alanine to proline, alanine to alanine, alanine to serine. A conservative mutation can be valine to methionine, valine to phenylalanine, valine to isoleucine, valine to leucine, valine to valine. A conservative mutation can be glycine to alanine, glycine to threonine, glycine to proline, glycine to serine, glycine to glycine. A conservative mutation can be Isoleucine to phenylalanine, isoleucine to isoleucine, isoleucine to valine, isoleucine to leucine, isoleucine to methionine. A conservative mutation can be phenylalanine to tryptophan, phenylalanine to phenylalanine, phenylalanine to tyrosine. A conservative mutation can be tyrosine to tryptophan, tyrosine to phenylalanine, tyrosine to tyrosine. A conservative mutation can be cysteine to serine, cysteine to threonine, cysteine to cysteine. A conservative mutation can be histidine to asparagine, histidine to lysine, histidine to glutamine, histidine to arginine, histidine to histidine. A conservative mutation can be glutamine to glutamic acid, glutamine to asparagine, glutamine to aspartic acid, glutamine to glutamine. A conservative mutation can be asparagine to glutamic acid, asparagine to asparagine, asparagine to aspartic acid, asparagine to glutamine. A conservative mutation can be lysine to asparagine, lysine to lysine, lysine to glutamine, lysine to arginine, lysine to histidine. A conservative mutation can be aspartic acid to glutamic acid, aspartic acid to asparagine, aspartic acid to aspartic acid, aspartic acid to glutamine. A conservative mutation can be glutamine to glutamine, glutamine to asparagine, glutamine to aspartic acid, glutamine to glutamine. A conservative mutation can be methionine to phenylalanine, methionine to isoleucine, methionine to valine, methionine to leucine, methionine to methionine. A conservative mutation can be tryptophan to tryptophan, tryptophan to phenylalanine, tryptophan to tyrosine.

Non-limiting examples of additional amino acid mutations can be: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

Any one of the aforementioned modifications, insertions, deletions, and/or mutations, can be made at any residue in an AAV sequence. The sequence may be a capsid sequence. In other cases, the sequence may not be a capsid sequence but rather a Rep and/or X sequence. The sequence may be in a VP1, VP2, and/or VP3 as previously described. In some cases, the sequence modification is of a loop of a capsid sequence, such as loop 3 and/or loop 4. In some cases, the modification is of a residue of a sequence in Table 1.

TABLE 1
Exemplary wildtype AAV capsid polypeptide sequences

| SEQ | AAV | |
|---|---|---|
| ID NO | Serotype | WT Polypeptide Sequence |
| 1 | AAV2 | MAADGYLPDWLEDTLSEGIRQWWKLKPGPPPPKPAERHKDDSR |
| | | GLVLPGYKYLGPFNGLDKGEPVNEADAAALEHDKAYDRQLDS |
| | | GDNPYLKYNHADAEFQERLKEDTSFGGNLGRAVFQAKKRVLEP |
| | | LGLVEEPVKTAPGKKRPVEHSPVEPDSSSGTGKAGQQPARKRLN |
| | | FGQTGDADSVPDPQPLGQPPAAPSGLGTNTMATGSGAPMADNN |
| | | EGADGVGNSSGNWHCDSTWMGDRVITTSTRTWALPTYNNHLY |
| | | KQISSQSGASNDNHYFGYSTPWGYFDFNRFHCHFSPRDWQRLIN |
| | | NNWGFRPKRLNFKLFNIQVKEVTQNDGTTTIANNLTSTVQVFTD |
| | | SEYQLPYVLGSAHQGCLPPFPADVFMVPQYGYLTLNNGSQAVG |
| | | RSSFYCLEYFPSQMLRTGNNFTFSYTFEDVPFHSSYAHSQSLDRL |
| | | MNPLIDQYLYYLSRTNTPSGTTTQSRLQFSQAGASDIRDQSRNW |
| | | LPGPCYRQQRVSKTSADNNNSEYSWTGATKYHLNGRDSLVNPG |
| | | PAMASHKDDEEKFFPQSGVLIFGKQGSEKTNVDIEKVMITDEEEI |
| | | RTTNPVATEQYGSVSTNLQRGNRQAATADVNTQGVLPGMVWQ |
| | | DRDVYLQGPIWAKIPHTDGHFHPSPLMGGFGLKHPPPQILIKNTP |
| | | VPANPSTTFSAAKFASFITQYSTGQVSVEIEWELQKENSKRWNPE |
| | | IQYTSNYNKSVNVDFTVDTNGVYSEPRPIGTRYLTRNL |
| 2 | AAV4 | MTDGYLPDWLEDNLSEGVREWWALQPGAPKPKANQQHQDNA |
| | | RGLVLPGYKYLGPGNGLDKGEPVNAADAAALEHDKAYDQQLK |
| | | AGDNPYLKYNHADAEFQQRLQGDTSFGGNLGRAVFQAKKRVL |
| | | EPLGLVEQAGETAPGKKRPLIESPQQPDSSTGIGKKGKQPAKKK |
| | | LVFEDETGAGDGPPEGSTSGAMSDDSEMRAAAGGAAVEGGQG |
| | | ADGVGNASGDWHCDSTWSEGHVTTTSTRTWVLPTYNNHLYKR |
| | | LGESLQSNTYNGFSTPWGYFDFNRFHCHFSPRDWQRLINNNWG |
| | | MRPKAMRVKIFNIQVKEVTTSNGETTVANNLTSTVQIFADSSYE |
| | | LPYVMDAGQEGSLPPFPNDVFMVPQYGYCGLVTGNTSQQQTDR |
| | | NAFYCLEYFPSQMLRTGNNFEITYSFEKVPFHSMYAHSQSLDRL |
| | | MNPLIDQYLWGLQSTTTGTTLNAGTATTNFTKLRPTNFSNFKKN |
| | | WLPGPSIKQQGFSKTANQNYKIPATGSDSLIKYETHSTLDGRWS |
| | | ALTPGPPMATAGPADSKFSNSQLIFAGPKQNGNTATVPGTLIFTS |
| | | EEELAATNATDTDMWGNLPGGDQSNSNLPTVDRLTALGAVPG |
| | | MVWQNRDIYYQGPIWAKIPHTDGHFHPSPLIGGFGLKHPPPQIFI |
| | | KNTPVPANPATTFSSTPVNSFITQYSTGQVSVQIDWEIQKERSKR |
| | | WNPEVQFTSNYGQQNSLLWAPDAAGKYTEPRAIGTRYLTHHL |
| 3 | AAV5 | MSFVDHPPDWLEEVGEGLREFLGLEAGPPKPKPNQQHQDQARG |
| | | LVLPGYNYLGPGNGLDRGEPVNRADEVAREHDISYNEQLEAGD |
| | | NPYLKYNHADAEFQEKLADDTSFGGNLGKAVFQAKKRVLEPFG |
| | | LVEEGAKTAPTGKRIDDHFPKRKKARTEEDSKPSTSSDAEAGPS |
| | | GSQQLQIPAQPASSLGADTMSAGGGGPLGDNNQGADGVGNAS |
| | | GDWHCDSTWMGDRVVTKSTRTWVLPSYNNHQYREIKSGSVDG |
| | | SNANAYFGYSTPWGYFDFNRFHSHWSPRDWQRLINNYWGFRP |
| | | RSLRVKIFNIQVKEVTVQDSTTTIANNLTSTVQVFTDDDYQLPYV |
| | | VGNGTEGCLPAFPPQVFTLPQYGYATLNRDNTENPTERSSFFCLE |
| | | YFPSKMLRTGNNFEFTYNFEEVPFHSSFAPSQNLFKLANPLVDQ |
| | | YLYRFVSTNNTGGVQFNKNLAGRYANTYKNWFPGPMGRTQG |
| | | WNLGSGVNRASVSAFATTNRMELEGASYQVPPQPNGMTNNLQ |
| | | GSNTYALENTMIFNSQPANPGTTATYLEGNMLITSESETQPVNR |
| | | VAYNVGGQMATNNQSSTTAPATGTYNLQEIVPGSVWMERDVY |
| | | LQGPIWAKIPETGAHFHPSPAMGGFGLKHPPPMMLIKNTPVPGNI |
| | | TSFSDVPVSSFITQYSTGQVTVEMEWELKKENSKRWNPEIQYTN |
| | | NYNDPQFVDFAPDSTGEYRTTRPIGTRYLTRPL |
| 4 | AAV6 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPFGL VEEGAKTAPGKKRPVEQSPQEPDSSSGIGKTGQQPAKK |
| | | RLNFGQTGDSESVPDPQPLGEPPATPAAVGPTTMASGGGAPMA |
| | | DNNEGADGVGNASGNWHCDSTWLGDRVITTSTRTWALPTYNN |
| | | HLYKQISSASTGASNDNHYFGYSTPWGYFDFNRFHCHFSPRDW |
| | | QRLINNNWGFRPKRLNFKLFNIQVKEVTTNDGVTTIANNLTSTV |
| | | QVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQ |
| | | SLDRLMNPLIDQYLYYLNRTQNQSGSAQNKDLLFSRGSPAGMS |
| | | VQPKNWLPGPCYRQQRVSKTKTDNNNSNFTWTGASKYNLNGR |
| | | ESIINPGTAMASHKDDKDKFFPMSGVMIFGKESAGASNTALDNV |
| | | MITDEEEIKATNPVATERFGTVAVNLQSSSTDPATGDVHVMGAL |
| | | PGMVWQDRDVYLQGPIWAKIPHTDGHFHPSPLMGGFGLKHPPP |
| | | QILIKNTPVPANPPAEFSATKFASFITQYSTGQVSVEIEWELQKEN |
| | | SKRWNPEVQYTSNYAKSANVDFTVDNNGLYTEPRPIGTRYLTR |
| | | PL |
| 5 | AAV11 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPLGLVEEGAKTAPGKKRPLESPQEPDSSSGIGKKGKQPARKR |
| | | LNFEEDTGAGDGPPEGSDTSAMSSDIEMRAAPGGNAVDAGQGS |
| | | DGVGNASGDWHCDSTWSEGKVTTTSTRTWVLPTYNNHLYLRL |
| | | GTTSSSNTYNGFSTPWGYFDFNRFHCHFSPRDWQRLINNNWGL |
| | | RPKAMRVKIFNIQVKEVTTSNGETTVANNLTSTVQIFADSSYELP |
| | | YVMDAGQEGSLPPFPNDVFMVPQYGYCGIVTGENQNQTDRNAF |
| | | YCLEYFPSQMLRTGNNFEMAYNFEKVPFHSMYAHSQSLDRLMN |
| | | PLLDQYLWHLQSTTSGETLNQGNAATTFGKIRSGDFAFYRKNW |
| | | LPGPCVKQQRFSKTASQNYKIPASGGNALLKYDTHYTLNNRWS |
| | | NIAPGPPMATAGPSDGDFSNAQLIFPGPSVTGNTTTSANNLLFTS |
| | | EEEIAATNPRDTDMFGQIADNNQNATTAPITGNVTAMGVLPGM |
| | | VWQNRDIYYQGPIWAKIPHADGHFHPSPLIGGFGLKHPPPQIFIK |
| | | NTPVPANPATTFTAARVDSFITQYSTGQVAVQIEWEIEKERSKR |
| | | WNPEVQFTSNYGNQSSMLWAPDTTGKYTEPRVIGSRYLTNHL |
| 92 | AAV12 | MAADGYLPDWLEDNLSEGIREWWALKPGAPQPKANQQHQDN |
| | | GRGLVLPGYKYLGPFNGLDKGEPVNEADAAALEHDKAYDKQL |
| | | EQGDNPYLKYNHADAEFQQRLATDTSFGGNLGRAVFQAKKRIL |
| | | EPLGLVEEGVKTAPGKKRPLEKTPNRPTNPDSGKAPAKKKQKD |
| | | GEPADSARRTLDFEDSGAGDGPPEGSSSGEMSHDAEMRAAPGG |
| | | NAVEAGQGADGVGNASGDWHCDSTWSEGRVTTTSTRTWVLPT |
| | | YNNHLYLRIGTTANSNTYNGFSTPWGYFDFNRFHCHFSPRDWQ |
| | | RLINNNWGLRPKSMRVKIFNIQVKEVTTSNGETTVANNLTSTVQ |
| | | IFADSTYELPYVMDAGQEGSFPPFPNDVFMVPQYGYCGVVTGK |
| | | NQNQTDRNAFYCLEYFPSQMLRTGNNFEVSYQFEKVPFHSMYA |
| | | HSQSLDRMMNPLLDQYLWHLQSTTTGNSLNQGTATTTYGKITT |
| | | GDFAYYRKNWLPGACIKQQKFSKNANQNYKIPASGGDALLKY |
| | | DTHTTLNGRWSNMAPGPPMATAGAGDSDFSNSQLIFAGPNPSG |
| | | NTTTSSNNLLFTSEEEIATTNPRDTDMFGQIADNNQNATTAPHIA |
| | | NLDAMGIVPGMVWQNRDIYYQGPIWAKVPHTDGHFHPSPLMG |
| | | GFGLKHPPPQIFIKNTPVPANPNTTFSAARINSFLTQYSTGQVAV |
| | | QIDWEIQKEHSKRWNPEVQFTSNYGTQNSMLWAPDNAGNYHE |
| | | LRAIGSRFLTHHL |

In some cases, a modification, such as insertion, deletion, and/or mutation is of a residue of a capsid polypeptide sequence in Table 1. In some cases, a modification is from 1-100, 100-200, 200-300, 300-400, 400-500, 500-600, 600-700, 700-800, or combinations thereof. In some cases, a modification is in a residue at position 200-300, 300-400, 400-500, 500-600 or combinations thereof. In some cases, a modification is in a residue at position 300-500 or combinations thereof. In an aspect, an insertion site is in the GH loop, or loop IV, of the AAV capsid protein, e.g., in a solvent-accessible portion of the GH loop, or loop IV, of the AAV capsid protein. For the GH loop, see, e.g., van Vliet et al. (2006) Mol. Ther. 14:809; Padron et al. (2005) J. Virol. 79:5047; and Shen et al. (2007) Mol. Ther. 15:1955. For example, the insertion site is within amino acids 570-611 of AAV2, within amino acids 571-612 of AAV1, within amino acids 560-601 of AAV5, within amino acids 571 to 612 of AAV6, within amino acids 572 to 613 of AAV7, within amino acids 573 to 614 of AAV8, within amino acids 571 to 612 of AAV9, or within amino acids 573 to 614 of AAV10.

For example, the insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 589 and 590 of AAV10. In some cases, a modification is at position 452, 453, 466, 467, 468, 471, 585, 586, 587, and/or 588 of AAV2. In some cases, a modification is at position 452 or 453 of AAV2. In some cases, a modification is at position 587 or 588 of AAV2. In some cases, a modification is an insertion at position 452, 453, 466, 467, 468, 471, 585, 586, 587, and/or 588 of SEQ ID NO: 1. In some cases, a modification is a mutation and the mutation is R585A or R588A of SEQ ID NO: 1.

In some embodiments, a subject modified AAV capsid does not include any other amino acid modifications mutations, substitutions, insertions, or deletions, other than an insertion of from about 5 amino acids to about 11 amino acids in a loop (loop 3 and/or 4) relative to a corresponding unmodified AAV capsid protein. In other embodiments, a subject variant AAV capsid includes from 1 to about 25 amino acid insertions, deletions, or substitutions, compared to an unmodified AAV capsid protein, in addition to an insertion of from about 5 amino acids to about 11 amino acids in the loop 3 and/or loop 4 relative to an unmodified AAV capsid protein. In an embodiment, a subject AAV virion capsid does not include any other amino acid substitutions, insertions, or deletions, other than an insertion of from about 7 amino acids to about 10 amino acids in a GH loop or loop IV relative to a corresponding parental AAV capsid protein. In other embodiments, a subject AAV virion capsid includes from 1 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein. For example, in some embodiments, a subject AAV virion capsid includes from 1 to about 5, from about 5 to about 10, from about 10 to about 15, from about 15 to about 20, or from about 20 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein.

In some cases, a chimeric AAV capsid is provided herein. A chimeric capsid comprises a polypeptide sequence from at least 2 AAV serotypes. A chimeric capsid can comprise a mix of sequences selected from serotypes AAV1, AAV2, AAV5, AAV4, AAV5, AAV6, AAV7, AAV8, AAV9, AAV10, AAV11, and/or AAV12. In some cases, the chimeric serotypes are different between VP1, VP2, and/or VP3. In some cases, a chimeric capsid comprises sequences from at least 2 serotypes selected from: AAV4 and AAV6, AAV5 and AAV6, AAV11 and AAV6, AAV12 and AAV6, and any combination thereof. In some cases, a first AAV serotype can be AAV4 and a second serotype can be AAV6. In some cases, a first AAV serotype and a second AAV serotype of a chimeric AAV vector can be AAV11 and AAV6. In some cases, a first AAV serotype and a second AAV serotype of a chimeric AAV vector can be AAV12 and AAV6. In some cases, a chimeric capsid comprises sequences from: AAV2 and AAV5 or AAV2 and AAV6. In some cases, a chimeric capsid comprises sequences from: AAV2 and AAV5, AAV2 and AAV6, AAV2 and AAV8, AAV2 and AAV9, AAV2 and AAV1, and AAV2 and AAV12.

The modifications to an AAV provided herein can confer enhanced activity to the modified AAV as compared to an otherwise unmodified or wildtype AAV. Modifications provided herein can improve cell transduction, tropism, and/or reduce immunogenicity associated with the capsid.

In some cases, a modification provided herein enhances cellular transduction. Cellular transduction can refer to the ability of an AAV to infect a cell (in vivo or in vitro) and/or deliver a transgene into the cell.

In some cases, a modification provided herein enhances tropism. Enhanced tropism refers to gaining the ability to transduce cells through an extra receptor, as compared to an otherwise unmodified AAV. In some aspects, enhanced tropism can improve infectivity of an ocular cell, thereby improving gene therapy by way utilization of the modified AAV. In some cases, a modification provided herein can improve tropism to an ocular cell selected from: bipolar, retinal ganglion, horizontal, amacrine, epithelial, retinal pigment, photoreceptor, or any combination thereof. In some cases, a modification improves tropism to a retinal cell.

Also provided herein are AAV vectors. AAV vectors comprise: inverted terminal repeats (ITRs), Rep, Cap, AAP, and X sequences. Typically, the AAV viral genome is flanked by the ITRs, which serve as packaging signal and origin of replication. The rep gene encodes a family of multifunctional proteins (Rep proteins) responsible for controlling viral transcription, replication, packaging, and integration in AAVS1. For AAV2, four Rep proteins are described. Expression of Rep78 and Rep68 is controlled by the AAV2-specific p5 promoter, while p19 controls expression of the smaller Rep proteins (Rep52 and Rep40). Rep68 and Rep40 are splice variants of Rep78 and Rep52, respectively. Numbers indicate the molecular weight. Expression of AAP and the viral capsid proteins VP1 (90 kDa), VP2 (72 kDa), and VP3 (60 kDa), all encoded in the cap gene, is controlled by the p40 promoter. The X gene is located at the 3′ end of the genome within a region shared with the cap gene and possesses its own promoter (p81). While the X protein seems to enhance viral replication, AAP is essential for capsid assembly. The three different VPs contribute in a 1 (VP1):1 (VP2):10 (VP3) ratio to the icosahedral AAV2 capsid.

A modified capsid protein disclosed herein can be isolated, e.g., purified. In some embodiments, a modified capsid disclosed herein is included in an AAV vector or an AAV virion (for example recombinant AAV virion rAAV). In other embodiments, such modified AAV vectors and/or AAV variant virions are used in an in vivo or ex vivo method of treating ocular disease in a primate retina, for example human retina.

Provided herein are also vectors that comprise modified AAV capsids. Any one of the previously described modifications can be encompassed in a vector provided herein. In some cases, an AAV vector comprises a modified capsid that comprises an exogenous sequence in at least two loops of a VP domain as compared to an otherwise comparable AAV capsid sequence that lacks the exogenous sequence. In some aspects, vectors provided herein can further comprise a transgene sequence.

Disclosed herein are also methods of modifying an AAV capsid from a WT capsid protein. An exemplary method of making a modified capsid is described in Example 2 herein. In some cases, a method of making a modified capsid that comprises a mutation comprises: subjecting a nucleic acid that comprises a nucleotide sequence encoding a WT capsid protein to a type of mutagenesis selected from the group consisting of: polymerase chain reaction mutagenesis, oligonucleotide-directed mutagenesis, saturation mutagenesis, loop-swapping mutagenesis, fragment shuffling mutagenesis, and a combination thereof. In some aspects, mutations in a subject AAV cap are generated using any known method. Suitable methods for mutagenesis of an AAV cap gene include, but are not limited to, a polymerase chain reaction (PCR)-based method, oligonucleotide-directed mutagenesis, and the like. Methods for generating mutations are well described in the art. See, e.g., Zhao et al. (1998) Nat. Biotechnol. 16:234-235; U.S. Pat. Nos. 6,579,678; 6,573,098; and 6,582,914.

In an aspect, mutant capsids disclosed herein can be generated through use of an AAV library and/or libraries. Such an AAV library or libraries is/are generated by mutating the cap gene, the gene which encodes the structural proteins of the AAV capsid, by a range of directed evolution methods, see e.g., Bartel et al. Am. Soc. Gene Cell Ther. 15th Annu. Meet. 20, S140 (2012); Bowles, D. et al. J. Virol. 77, 423-432 (2003); Gray et al. Mol. Ther. 18, 570-578 (2010); Grimm, D. et al. J. Virol. 82, 5887-5911; Koerber, J. T. et al. Mol. Ther. 16, 1703-1709 (2008); Li W. et al. Mol. Ther. 16, 1252-1260 (2008); Koerber, J. T. et al. Methods Mol. Biol. 434, 161-170 (2008); Koerber, J. T. et al. Hum. Gene Ther. 18, 367-378 (2007); and Koerber, J. T. et al. Mol. Ther. 17, 2088-2095 (2009). Such techniques, without limitation, are as follows: i) Error-prone PCR to introduce random point mutations into the AAV cap open reading frame (ORF) at a predetermined, modifiable rate; ii) In vitro or in vivo viral recombination or “DNA shuffling” to generate random chimeras of AAV cap genes to yield a gene library with multiple AAV serotypes; iii) Random peptide insertions at defined sites of the capsid by ligation of degenerate oligonucleotides in the cap ORF; iv) Defined insertions of peptide-encoding sequences into random locations of the AAV cap ORF using transposon mutagenesis; v) Replacing surface loops of AAV capsids with libraries of peptide sequences bioinformationally designed based on the level of conservation of each amino acid position among natural AAV serotypes and variants to generate “loop-swap” libraries; vi) Random amino acid substitution at positions of degeneracy between AAV serotypes to generate libraries of ancestral variants (Santiago-Ortiz et al, 2015); and a combination of such techniques thereof.

In some embodiments, a modified capsid can be generated using a staggered extension process. The staggered extension process involves amplification of the cap gene using a PCR-based method. The template cap gene is primed using specific PCR primers, followed by repeated cycles of denaturation and very short annealing/polymerase-catalyzed extension. In each cycle, the growing fragments anneal to different templates based on sequence complementarity and extend further. The cycles of denaturation, annealing, and extension are repeated until full-length sequences form. The resulting full-length sequences include at least one mutation in the cap gene compared to a wild-type AAV cap gene. The PCR products comprising AAV cap sequences that include one or more mutations are inserted into a plasmid containing a wild-type AAV genome. The result is a library of AAV cap mutants. Thus, the disclosure provides a mutant AAV cap gene library comprising from about 10 to about 10¹⁰ members, and comprising mutations in the AAV cap gene. A given member of the library has from about one to about 50 mutations in the AAV cap gene. A subject library comprises from 10 to about 10⁹ distinct members, each having a different mutation(s) in the AAV cap gene. Once a cap mutant library is generated, viral particles are produced that can then be selected on the basis of altered capsid properties. Library plasmid DNA is transfected into a suitable subject host cell (e.g., 293 cells and/or ARPE-19 cells), followed by introduction into the cell of helper virus. Viral particles produced by the transfected host cells (“AAV library particles) are collected.

In an aspect, once an AAV library or libraries is/are generated, virions are then packaged, such that each AAV particle is comprised of a modified capsid surrounding a cap gene encoding that capsid, and purified. In some cases, variants, those comprising a modification, of the library are then subjected to in vitro and/or in vivo selective pressure techniques known by and readily available to the skilled artisan in the field of AAV. See e.g., Maheshri, N. et al. Nature Biotech. 24, 198-204 (2006); Dalkara, D. et al. Sci. Transl. Med. 5, 189ra76 (2013); Lisowski, L. et al. Nature. 506, 382-286 (2013); Yang, L. et al. PNAS. 106, 3946-3951 (2009); Gao, G. et al. Mol. Ther. 13, 77-87 (2006); and Bell, P. et al. Hum. Gene. Ther. 22, 985-997 (2011). For example, without limitation, modified AAVs can be selected using i) affinity columns in which elution of different fractions yields variants with altered binding properties; ii) primary cells—isolated from tissue samples or immortal cell lines that mimic the behavior of cells in the human body—which yield AAV variants with increased efficiency and/or tissue specificity; iii) animal models—which mimic a clinical gene therapy environment—which yield AAV variants that have successfully infected target tissue; iv) human xenograft models which yield AAV variants comprising subject modifications that have infected grafted human cells; and/or a combination of selection techniques thereof. Once virions are selected, they may be recovered by known techniques such as, without limitation, adenovirus-mediated replication, PCR amplification, Next Generation sequencing and cloning, and the like. Virus clones are then enriched through repeated rounds of the selection techniques and AAV DNA is isolated to recover selected variant cap genes of interest. Such selected variants can be subjected to further modification or mutation and as such serve as a new starting point for further selection steps to iteratively increase AAV viral fitness. However, in certain instances, successful capsids have been generated without additional mutation.

Subject modified AAVs disclosed herein can be generated through the use of in vivo directed evolution involving the use of primate retinal screens following intravitreal administration. In some embodiments, modified capsid proteins disclosed herein, when present in an AAV virion, confer increased transduction of a retinal cell compared to the transduction of the retinal cell by an AAV virion comprising the corresponding parental AAV capsid protein or wild-type AAV. For example, in some embodiments, the variant capsid proteins disclosed herein, when present in an AAV virion, confer more efficient transduction of primate retinal cells than AAV virions comprising the corresponding parental AAV capsid protein or wild-type AAV capsid protein, e.g. the retinal cells take up more AAV virions comprising the subject variant AAV capsid protein than AAV virions comprising the parental AAV capsid protein or wild-type AAV.

Provided herein is also a method for treating a disease or condition in a subject in need thereof. Subject methods can comprise administering a therapeutically effective amount of a pharmaceutical composition that comprises an adeno-associated virus (AAV) vector that comprises a modified capsid that comprises an exogenous polypeptide sequence in a VP domain of the AAV capsid as compared to an otherwise comparable unmodified AAV capsid, the exogenous polypeptide sequence comprising a sequence of formula 1: X0-X1-X2-X1-X3-X1-X1-X4 (SEQ ID NO: 108), wherein X0 is Valine (V), Isoleucine (I), Leucine (L), Phenylalanine (F), Tryptophan (W), Tyrosine (Y) or Methionine (M), wherein X1 is Alanine (A), Asparagine (N), Glutamine (Q), Serine (S), Threonine (T), Glutamic Acid (E), Aspartic Acid (D), Lysine (K), Arginine (R), or Histidine (H), wherein X2 is V, I, L, or M, wherein X3 is E, S, or Q, and wherein X4 is K, R, E, or A, optionally wherein formula 1 further comprises X5 being Proline (P) or R.

In some cases, a subject AAV vector comprises a sequence that comprises at least 60%, 70%, 80%, 85%, 90%, 95%, 96%, 97%, 98%, or 99% identity to SEQ ID NO: 28-SEQ ID NO: 47. In an embodiment, the AAV vector comprises the modified capsid of SEQ ID NO: 34.

Also provided herein are engineered AAV virions that comprise a modified AAV capsid. Engineered AAV virions can be generated utilizing a host or “producer” cell for rAAV vector replication and packaging. Such a producer cell (usually a mammalian host cell) generally comprises or is modified to comprise several different types of components for rAAV production. The first component is a recombinant adeno-associated viral (rAAV) vector genome (or “engineered AAV”) that can be replicated and packaged into vector particles by the host packaging cell. The rAAV pro-vector can comprise a subject transgene with which it is desired to genetically alter another cell in the context of gene therapy (since the packaging of such a transgene into rAAV vector particles can be effectively used to deliver the transgene to a variety of mammalian cells). The transgene is generally flanked by two AAV inverted terminal repeats (ITRs) which comprise sequences that are recognized during excision, replication and packaging of the AAV vector, as well as during integration of the vector into a host cell genome.

Another component can be a helper virus that can provide helper functions for AAV replication. Although adenovirus is commonly employed, other helper viruses can also be used as is known in the art. Alternatively, the requisite helper virus functions can be isolated genetically from a helper virus and the encoding genes can be used to provide helper virus functions in trans. The AAV vector elements and the helper virus (or helper virus functions) can be introduced into the host cell either simultaneously or sequentially in any order.

Additional components for AAV production to be provided in the producer cell are “AAV packaging genes” such as AAV rep and cap genes that provide replication and encapsidation proteins, respectively. Several different versions of AAV packaging genes can be provided (including rep-cap cassettes and separate rep and/or cap cassettes in which the rep and/or cap genes can be left under the control of the native promoters or operably linked to heterologous promoters. Such AAV packaging genes can be introduced either transiently or stably into the host packaging cell, as is known in the art and described in more detail below.

The disclosure herein further provides host cells such as, without limitation, isolated (genetically modified) host cells comprising a subject nucleic acid. A host cell according to the invention disclosed herein, can be an isolated cell, such as a cell from an in vitro cell culture. Such a host cell is useful for producing a subject modified AAV virion, as described herein. In one embodiment, such a host cell is stably genetically modified with a nucleic acid. In other embodiments, a host cell is transiently genetically modified with a nucleic acid. Such a nucleic acid is introduced stably or transiently into a host cell, using established techniques, including, but not limited to, electroporation, calcium phosphate precipitation, liposome-mediated transfection, and the like. For stable transformation, a nucleic acid will generally further include a selectable marker, e.g., any of several well-known selectable markers such as neomycin resistance, and the like. Such a host cell is generated by introducing a nucleic acid into any of a variety of cells, e.g., mammalian cells, including, e.g., murine cells, and primate cells (e.g., human cells). Exemplary mammalian cells include, but are not limited to, primary cells and cell lines, where exemplary cell lines include, but are not limited to, 293 cells, COS cells, HeLa cells, Vero cells, 3T3 mouse fibroblasts, C3H10T1/2 fibroblasts, CHO cells, and the like. Exemplary host cells include, without limitation, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. A host cell can also be made using a baculovirus to infect insect cells such as Sf9 cells, which produce AAV (see, e.g., U.S. Pat. No. 7,271,002; U.S. patent application Ser. No. 12/297,958). In some embodiments, a genetically modified host cell includes, in addition to a nucleic acid comprising a nucleotide sequence encoding a variant AAV capsid protein, as described above, a nucleic acid that comprises a nucleotide sequence encoding one or more AAV rep proteins. In other embodiments, a host cell further comprises subject vector. A subject modified virion can be generated using such host cells. Methods of generating virions are described in, e.g., U.S. Patent Publication No. 2005/0053922 and U.S. Patent Publication No. 2009/0202490.

A subject engineered AAV virion, which in some cases comprises a transgene, can be produced using methodology, known to those of skill in the art. The methods generally involve the steps of (1) introducing a subject engineered AAV vector into a host cell; (2) introducing an AAV helper construct into the host cell, where the helper construct includes AAV coding regions capable of being expressed in the host cell to complement AAV helper functions missing from the AAV vector; (3) introducing one or more helper viruses and/or accessory function vectors into the host cell, wherein the helper virus and/or accessory function vectors provide accessory functions capable of supporting efficient recombinant AAV (“rAAV”) virion production in the host cell; and (4) culturing the host cell to produce engineered AAV virions. The AAV expression vector, AAV helper construct and the helper virus or accessory function vector(s) can be introduced into the host cell, either simultaneously or serially, using standard transfection techniques. AAV expression vectors are constructed using known techniques to at least provide as operatively linked components in the direction of transcription, control elements including a transcriptional initiation region, the DNA of interest and a transcriptional termination region. The control elements can be selected to be functional in a mammalian muscle cell. The resulting construct which contains the operatively linked components cab be bounded (5′ and 3′) with functional AAV ITR sequences.

In some cases, known nucleotide sequences of AAV ITRs can be utilized, See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. In some aspects, AAV ITRs used in the compositions and methods provided herein need not have a wild-type nucleotide sequence, and may be modified, e.g., by the insertion, deletion or substitution of nucleotides. Additionally, AAV ITRs may be derived from any of several AAV serotypes, provided herein, such as including without limitation, AAV-1, AAV-2, AAV-3, AAV-4, AAV-5, AAV-7, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell. ITRs allow replication of the vector sequence in the presence of an appropriate mixture of Rep proteins. ITRs also allow for the incorporation of the vector sequence into the capsid to generate an AAV particle.

The present disclosure provides adeno-associated virus (AAV) virions with altered capsid protein, where the AAV virions exhibit greater infectivity of an ocular cell, as compared to an unmodified AAV, such as a wild-type AAV, when administered via intravitreal injection. The present disclosure further provides methods of delivering a transgene to a cell, such as a retinal cell, and methods of treating ocular disease. The retinal cell can be a photoreceptor (e.g., rods; cones), a retinal ganglion cell (RGC), a Müller cell (a Müller glial cell), a bipolar cell, an amacrine cell, a horizontal cell, or a retinal pigmented epithelium (RPE) cell.

Provided herein is a method for treating a disease or condition in a subject in need thereof, the method comprising administering a therapeutically effective amount of a pharmaceutical composition that comprises an adeno-associated virus (AAV) vector that comprises a modified capsid. In some cases, the modified capsid comprises an exogenous polypeptide sequence in at least two loops of a VP domain as compared to an otherwise comparable AAV capsid sequence that lacks the exogenous polypeptide sequence. In some aspects, the exogenous polypeptide sequence comprises a sequence of Table 2. In some cases, a subject AAV vector further comprises a sequence that comprises a transgene. Also provided is a method for treating a disease or condition in a subject in need thereof, the method comprising administering a therapeutically effective amount of a pharmaceutical composition that comprises an adeno-associated virus (AAV) vector. A subject AAV vector can comprise: (a) a modified capsid that comprises an exogenous sequence in at least two loops of a VP domain as compared to an otherwise comparable AAV capsid sequence that lacks the exogenous sequence; and/or (b) a transgene. In some cases, the vector, upon contacting with a plurality of cells, has at least 3 fold, 4-fold, 5-fold, 10-fold, 15-fold, 30-fold, 60-fold, or 100-fold increased expression of the transgene post transfection in the plurality of cells as compared to contacting the plurality of cells with an otherwise comparable AAV vector that lacks (a). In some cases, the increased expression comprises at least a 5-fold, 10-fold, 20-fold, 50-fold, 100-fold, 200-fold, or 500-fold increase as compared to the contacting the plurality of cells with the otherwise comparable AAV vector that lacks (a).

Provided herein is also a composition that comprises a plurality of a subject AAV virion. Also provided is an engineered cell generated by transfecting a cell with a subject AAV vector of or a subject AAV virion. In some aspects, viral particles can be isolated from a subject engineered cell. Provided can also be a plurality of adeno-associated viral (AAV) particles isolated from the engineered cell. Additionally, a composition is provided that comprises the adeno-associated viral particles in unit dosage form. In some cases, a subject composition can be cryopreserved.

Provided herein are also transgenes. Suitable transgenes include, but are not limited to, those encoding proteins used for the treatment of disease. In some aspects a disease is an ocular disease. In another example, a suitable disease can be a retinal disease. In some aspects, a transgene can encode for a therapeutic, such as an ocular therapeutic. Ocular therapeutics can be effective to reduce at least a symptom of a disease, such as an ocular disease. In some aspects, a therapeutic can be effective to reduce at least a symptom of a retinal disease, treat a retinal disease, or eliminate a retinal disease. Suitable ocular therapeutics can refer to antibodies or biologically active fragments thereof and/or biologics. Targets of antibodies and biologics can comprise PDGF-BB, C5 complement, C3 complement, TNF-alpha, VEGF-A, VEGFR01, DDIT4, KSP, PEDF, VEGF, VEGFL, thrombospondin-1, CD47, alpha 5 beta 1 integrin, endostatis angiostatin, pathologic blood vessels, or any combination thereof.

In some cases, an ocular therapeutic is an antibody or the biologically active fragment thereof. An antibody can be a monoclonal antibody. In some cases, an antibody can be fully human or humanized. Suitable antibodies can be anti-VEGF, anti-VEGFL, anti-thrombospondin-1, anti-CD47, anti-TNF-alpha, anti-CD20, anti-CD52, and anti-CD11a, anti-complement 5, and/or anti-complement 3. In some cases, an antibody or biologically active fragment comprises: rituximab, infliximab, ranibizumab, bevacizumab, JSM6427, and/or conbercept.

In another embodiment, an antibody is a single-chain version of Ranibizumab (sc-Ranibizumab). Ranibizumab is a monoclonal IgG1 antibody fragment (Fab) that binds to and blocks all isoforms of VEGF-A. Ranibizumab is expressed in bacteria as two separate chains (light and heavy) which are joined by a disulfide bond between the constant light (CL) and constant heavy 1 (CH1) domain. The approved dose of intravitreal Ranibizumab is either 0.3 or 0.5 mg in 0.05 mL depending on the indication. Ranibizumab is approved for the treatment of wet age-related macular degeneration, macular edema following retinal vein occlusion, diabetic macular edema and diabetic retinopathy.

In some cases, an ocular therapeutic is a biologic. In some aspects, a biologic is selected from macromolecules such as a protein, peptide, aptamer, and/or non-translated RNAs, such as an antisense RNA, a ribozyme, an RNAi and an siRNA. In some aspects, a composition provided herein, such as an AAV virion that comprises a modified capsid, comprises a transgene encoding a therapeutic. In some embodiments, the therapeutic is an interfering RNA. In some embodiments, the therapeutic is an aptamer. In some embodiments, the therapeutic is a polypeptide. In some embodiments, the therapeutic is a site-specific nuclease that provide for site-specific knock-down of gene function.

In some cases, a biologic is a DNA aptamer, RNA aptamer, dual siRNA, gene, polypeptide, or protein scaffold. In some cases, a biologic is selected from: Lipoprotein Lipase, Retinoid Isomerohydrolase RPE65, or complement H. In some cases, a biologic comprises a siRNA that targets VEGF-A, for example Bevasiranib. In some cases, a biologic is an RNA aptamer, such as Pegaptanib. In some cases, a biologic is a polypeptide such as a fusion protein, such as aflibercept. In some cases, a biologic is a DNA aptamer, such as Fovista. In some cases, a biologic is a protein scaffold such as DARPins.

In an aspect, a biologic is aflibercept. Aflibercept can be a recombinant fusion protein comprising extracellular domains of human VEGF receptors 1 and 2 fused to the Fc portion of human IgG1. Aflibercept acts as a soluble decoy receptor that binds VEGF-A and PDGF with greater affinity than the native receptors. The approved dose of intravitreal Aflibercept injection is 2.0 mg, the dosing of which varies according to indication. Aflibercept is indicated for the treatment of neovascular (wet) age-related macular degeneration, macular edema following retinal vein occlusion, diabetic macular edema and diabetic retinopathy.

In some aspects, a biologic therapeutic is an aptamer, exemplary aptamers of interest include an aptamer against vascular endothelial growth factor (VEGF). See, e.g., Ng et al. (2006) Nat. Rev. Drug Discovery 5:123; and Lee et al. (2005) Proc. Natl. Acad. Sci. USA 102:18902. For example, a VEGF aptamer can comprise the nucleotide sequence 5′-cgcaaucagugaaugcuuauacauccg-3′ (SEQ ID NO: 109). Also suitable for use is a PDGF-specific aptamer, e.g., E10030; see, e.g., Ni and Hui (2009) Ophthalmologica 223:401; and Akiyama et al. (2006) J. Cell Physiol. 207:407).

In some aspects, a biologic therapeutic is an interfering RNA (RNAi), suitable RNAi include RNAi that can reduce a level of an apoptotic or angiogenic factor in a cell. For example, an RNAi can be an shRNA or siRNA that reduces the level of a gene product that induces or promotes apoptosis in a cell. Genes whose gene products induce or promote apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730. Interfering RNAs could also be against an angiogenic product, for example VEGF (e.g., Cand5; see, e.g., U.S. Patent Publication No. 2011/0143400; U.S. Patent Publication No. 2008/0188437; and Reich et al. (2003) Mol. Vis. 9:210), VEGFR1 (e.g., Sirna-027; see, e.g., Kaiser et al. (2010) Am. J. Ophthalmol. 150:33; and Shen et al. (2006) Gene Ther. 13:225), or VEGFR2 (Kou et al. (2005) Biochem. 44:15064). See also, U.S. Pat. Nos. 6,649,596, 6,399,586, 5,661,135, 5,639,872, and 5,639,736; and 7,947,659 and 7,919,473.

In some cases, a biologic comprises a polypeptide. A polypeptide can enhance function of a retinal cell, e.g., the function of a rod or cone photoreceptor cell, a retinal ganglion cell, a Müller cell, a bipolar cell, an amacrine cell, a horizontal cell, or a retinal pigmented epithelial cell. Exemplary polypeptides include neuroprotective polypeptides (e.g., GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); endostatin; tumstatin; angiostatin; a soluble Flt polypeptide (Lai et al. (2005) Mol. Ther. 12:659); an Fc fusion protein comprising a soluble Flt polypeptide (see, e.g., Pechan et al. (2009) Gene Ther. 16:10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-X1); and the like. Suitable polypeptides include, but are not limited to, glial derived neurotrophic factor (GDNF); fibroblast growth factor 2; neurturin (NTN); ciliary neurotrophic factor (CNTF); nerve growth factor (NGF); neurotrophin-4 (NT4); brain derived neurotrophic factor (BDNF; epidermal growth factor; rhodopsin; X-linked inhibitor of apoptosis; and Sonic hedgehog.

In some cases, a polypeptide can comprise retinoschisin, retinitis pigmentosa GTPase regulator (RGPR)-interacting protein-1 (see, e.g., GenBank Accession Nos. Q96KN7, Q9EPQ2, and Q9GLM3, peripherin-2 (Prph2) (see, e.g., GenBank Accession No. NP_000313, peripherin, a retinal pigment epithelium-specific protein (RPE65), (see, e.g., GenBank AAC39660; and Morimura et al. (1998) Proc. Natl. Acad. Sci. USA 95:3088), CHM (choroidermia (Rab escort protein 1)), a polypeptide that, when defective or missing, causes choroideremia (see, e.g., Donnelly et al. (1994) Hum. Mol. Genet. 3:1017; and van Bokhoven et al. (1994) Hum. Mol. Genet. 3:1041); and Crumbs homolog 1 (CRB1), a polypeptide that, when defective or missing, causes Leber congenital amaurosis and retinitis pigmentosa (see, e.g., den Hollander et al. (1999) Nat. Genet. 23:217; and GenBank Accession No. CAM23328). Suitable polypeptides also include polypeptides that, when defective or missing, lead to achromotopsia, where such polypeptides include, e.g., cone photoreceptor cGMP-gated channel subunit alpha (CNGA3) (see, e.g., GenBank Accession No. NP_001289; and Booij et al. (2011) Ophthalmology 118:160-167); cone photoreceptor cGMP-gated cation channel beta-subunit (CNGB3) (see, e.g., Kohl et al. (2005) Eur J Hum Genet. 13(3):302); guanine nucleotide binding protein (G protein), alpha transducing activity polypeptide 2 (GNAT2) (ACHM4); and ACHM5; and polypeptides that, when defective or lacking, lead to various forms of color blindness.

In some cases, a biologic comprises a site-specific endonuclease that provide for site-specific knock-down of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., a subject AAV virion can be used to deliver both a site-specific endonuclease that knocks out a defective allele, and can be used to deliver a functional copy of the defective allele, resulting in repair of the defective allele, thereby providing for production of a functional retinal protein (e.g., functional retinoschisin, functional RPE65, functional peripherin, etc.). See, e.g., Li et al. (2011) Nature 475:217. In some embodiments, a subject AAV virion comprises a transgene that encodes a site-specific endonuclease; and a heterologous nucleotide sequence that encodes a functional copy of a defective allele, where the functional copy encodes a functional retinal protein. Functional retinal proteins include, e.g., retinoschisin, RPE65, retinitis pigmentosa GTPase regulator (RGPR)-interacting protein-1, peripherin, peripherin-2, and the like. Site-specific endonucleases that are suitable for use include, e.g., CRISPR, zinc finger nucleases (ZFNs); and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073.

In some embodiments, cell type-specific or a tissue-specific promoter can be operably linked to a transgene encoding for a subject therapeutic, such that the gene product is produced selectively or preferentially in a particular cell type(s) or tissue(s). In some embodiments, an inducible promoter will be operably linked to a transgene sequence. In some cases, a promoter can be operably linked to a photoreceptor-specific regulatory element (e.g., a photoreceptor-specific promoter), e.g., a regulatory element that confers selective expression of the operably linked gene in a photoreceptor cell. Suitable photoreceptor-specific regulatory elements include, e.g., a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225) and the like.

In some embodiments, a biologic delivered by a subject modified AAV can act to inhibit angiogenesis. In certain preferred embodiments, the biologic delivered by a subject modified AAV can act to inhibit the activity of one or more mammalian VEGF proteins selected from the group consisting of VEGF-A, VEGF-B, VEGF-C, VEGF-D and PDGF. In particularly preferred embodiments, the biologic delivered by the subject AAV variants inhibit the activity of VEGF-A. VEGF-A has 9 isoforms generated by alternative splicing, the most physiologically relevant of which is VEGF 165. VEGF-A levels have been found to be elevated in the vitreous of patients with wet age-related macular degeneration, diabetic macular edema and retinal vein occlusion. Gene product(s) which inhibit the activity of VEGF-A in the eye and which are therefore effective to treat patients with elevated vitreous VEGF-A include, but are not limited to, Aflibercept, Ranibizumab, Brolucizumab, Bevacizumab, and soluble fms-like tyrosine kinase 1 (sFLT1) (GenBank Acc. No. U01134). In some embodiments, an infectious AAV virion is provided comprising (i) a variant AAV capsid protein as herein described and (ii) a transgene comprising a VEGF inhibitor. In an embodiment, a transgene comprises multiple sequences, each of which encodes a distinct VEGF-A inhibitor. In an embodiment, the transgene can be Aflibercept.

Provided compositions and methods herein can be sufficient to enhance delivery of subject transgene by at least about 3%, 5%, 10%, 15%, 20%, 25%, 30%, 35%, 40%, 45%, 50%, 55%, 60%, 65%, 70%, 75%, 80%, 85% 90%, 91%, 92%, 93%, 94%, 95%, 96%, 97%, 98%, 99% or up to 100% more than an otherwise comparable unmodified AAV, AAV capsid for instance. In some cases, modifications can be sufficient to enhance delivery of subject transgenes by at least about 1-fold, 6-fold, 11-fold, 16-fold, 21-fold, 26-fold, 31-fold, 36-fold, 41-fold, 46-fold, 51-fold, 56-fold, 61-fold, 66-fold, 71-fold, 76-fold, 81-fold, 86-fold, 91-fold, 96-fold, 101-fold, 106-fold, 111-fold, 116-fold, 121-fold, 126-fold, 131-fold, 136-fold, 141-fold, 146-fold, 151-fold, 156-fold, 161-fold, 166-fold, 171-fold, 176-fold, 181-fold, 186-fold, 191-fold, 196-fold, 201-fold, 206-fold, 211-fold, 216-fold, 221-fold, 226-fold, 231-fold, 236-fold, 241-fold, 246-fold, 251-fold, 256-fold, 261-fold, 266-fold, 271-fold, 276-fold, 281-fold, 286-fold, 291-fold, 296-fold, 301-fold, 306-fold, 311-fold, 316-fold, 321-fold, 326-fold, 331-fold, 336-fold, 341-fold, 346-fold, or 350-fold more than an otherwise comparable unmodified AAV, for example AAV capsid.

A subject AAV virion can exhibit at least 1-fold, 6-fold, 10-fold, 15-fold, 20-fold, 25-fold, at least 50-fold, or more than 50-fold, increased infectivity of a retinal cell, compared to the infectivity of the retinal cell (photoreceptor, ganglion cell, RPE cell, amacrine cell, horizontal cell, muller cell, and the like) by an AAV virion comprising an otherwise comparable WT AAV capsid protein.

In some embodiments, a subject modified composition, such as an AAV virion, selectively infects a retinal cell, with 10-fold, 15-fold, 20-fold, 25-fold, 50-fold, or more than 50-fold, specificity than a non-retinal cell, e.g., a cell outside the eye. For example, in some embodiments, a subject AAV virion selectively infects a retinal cell, e.g., a subject engineered AAV virion infects a photoreceptor cell with 10-fold, 15-fold, 20-fold, 25-fold, 50-fold, or more than 50-fold, specificity than a non-retinal cell, e.g., a cell outside the eye. In an embodiment, a subject AAV virion exhibits at least 10-fold, at least 15-fold, at least 20-fold, at least 25-fold, at least 50-fold, or more than 50-fold, increased infectivity of a retinal cell, compared to the infectivity of the retinal cell by an AAV virion comprising an otherwise comparable AAV capsid protein. In some cases, a subject AAV virion exhibits at least at least 15-fold, at least 20-fold, at least 25-fold, at least 50-fold, or more than 50-fold, increased infectivity of a retinal cell, when administered via intravitreal injection, compared to the infectivity of the retinal cell by an otherwise comparable unmodified AAV virion comprising the corresponding parental AAV capsid protein, when administered via intravitreal injection. In an embodiment, a subject AAV virion exhibits at least 10-fold, at least 15-fold, at least 20-fold, at least at least 50-fold, or more than 50-fold, increased infectivity of a photoreceptor (rod or cone) cell, compared to the infectivity of the photoreceptor cell by an AAV virion comprising the corresponding parental AAV capsid protein. In some embodiments, a subject AAV virion exhibits at least 10-fold, at least 15-fold, at least 20-fold, at least 25-fold, at least 50-fold, or more than 50-fold, increased infectivity of a photoreceptor (rod or cone) cell, when administered via intravitreal injection, compared to the infectivity of the photoreceptor cell by an AAV virion comprising the corresponding parental AAV capsid protein, when administered via intravitreal injection. In some embodiments, a subject AAV virion exhibits at least 10-fold, at least 15-fold, at least 20-fold, at least 25-fold, at least 50-fold, or more than 50-fold, increased infectivity of an RGC, compared to the infectivity of the RGC by an AAV virion comprising the corresponding parental AAV capsid protein. In some embodiments, a subject AAV virion exhibits at least 10-fold, at least 15-fold, at least 20-fold, at least 25-fold, at least 50-fold, or more than 50-fold, increased infectivity of an RGC, when administered via intravitreal injection, compared to the infectivity of the RGC by an AAV virion comprising the corresponding parental AAV capsid protein, when administered via intravitreal injection.

In an aspect, transduction, infectivity, and/or tropism is detected by determining the presence of a transgene in an infected cell in vitro. An increase in transduction of a retinal cell, e.g. increased efficiency of transduction, broader transduction, more preferential transduction, etc. may be readily assessed in vitro or in vivo by any number of methods in the art for measuring gene expression. For example, the AAV may be packaged with a genome comprising an expression cassette comprising a reporter gene, e.g. a fluorescent protein, under the control of a ubiquitous or tissue specific promoter, and the extent of transduction assessed by detecting the fluorescent protein by, e.g., fluorescence microscopy. As another example, the AAV may be packaged with a genome comprising a bar coded nucleic acid sequence, and the extent of transduction assessed by detecting the nucleic acid sequence by, e.g., PCR. As another example, the AAV may be packaged with a genome comprising an expression cassette comprising a therapeutic gene for the treatment of a retinal disease, and the extent of transduction assessed by detecting the treatment of the retinal disease in an afflicted patient that was administered the AAV. For example, cells can be transduced utilizing a modified capsid AAV composition described herein and the presence of the transgene can be determined via microscopy, flow cytometry, PCR-based assays, ELISA, histology, or any combination thereof.

The present disclosure further provides methods of delivering a transgene to a subject in need thereof. The methods generally involve introducing a subject composition such as a composition comprising a modified AAV capsid, including but not limited to an AAV virion, an AAV vector, or combinations thereof to an individual.

Transduced cells and/or AAV virions that, for example that show transduction potential, can then be formulated into pharmaceutical compositions, described more fully below, and the composition introduced into the subject by various techniques, such as by intravitreally, intramuscular, intravenous, subcutaneous, and/or intraperitoneal injection.

For in vivo delivery, subject AAV virions can be formulated into pharmaceutical compositions and will generally be administered intravitreally or parenterally (e.g., administered via an intramuscular, subcutaneous, intratumoral, transdermal, intrathecal, etc., route of administration.)

The compositions described throughout can be formulated into a pharmaceutical composition. In some aspects, a pharmaceutical composition can be used to treat a subject such as a human or mammal, in need thereof. In some cases, a subject can be diagnosed with a disease, e.g., ocular disease. In some aspects, subject pharmaceutical compositions are co-administered with secondary therapies.

Provided herein can also be pharmaceutical compositions that comprise any of the previously described compositions. A pharmaceutical composition can be in unit dose form. In an aspect, a pharmaceutical composition provided herein comprises a modified AAV capsid.

In an aspect, the present disclosure provides a pharmaceutical composition comprising: a) a subject AAV virion, as described above; and b) a pharmaceutically acceptable carrier, diluent, excipient, or buffer. In some embodiments, the pharmaceutically acceptable carrier, diluent, excipient, or buffer is suitable for use in a human.

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

Pharmaceutical compositions provided herein can be utilized to prevent and/or treat a disease. In some aspects, a disease is an ocular disease. In some cases, an ocular disease is a juvenile disease. In some cases, an ocular disease is of advanced age. In some cases, an ocular disease is of the retina. Ocular diseases that can be prevented and/or treated with pharmaceutical compositions and methods provided herein are: Achromatopsia, neovascularization related retinal disorder such as Age-related macular degeneration (AMD), wet-Age-related macular degeneration (wAMD), Geographic atrophy (GA), Diabetic retinopathy (DR), Diabetic macular edema (DME), Glaucoma, Bardet-Biedl Syndrome, Best Disease, Choroideremia, Leber Congenital Amaurosis, Leber Hereditary Optic Neuropathy (LHON), Macular degeneration, Polypoidal choroidal vasculopathy (PCV), Retinitis pigmentosa, Refsum disease, Stargardt disease, Usher syndrome, X-linked retinoschisis (XLRS), Inherited Retinal Disease (IRD), Rod-cone dystrophy, Cone-rod dystrophy, Oguchi disease, Malattia Leventinese (Familial Dominant Drusen), Blue-cone monochromacy, retina vein occlusion (RVO), and Uveitic Macular Oedema (UMO). In some cases, a retinal disease is AMD. In some cases, a retinal disease is wet AMD. In some cases, a retinal disease is dry AMD. Additional ocular disease comprise: acute macular neuroretinopathy; Behcet's disease; choroidal neovascularization; diabetic uveitis; histoplasmosis; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy; photocoagulation, radiation retinopathy; epiretinal membrane disorders; branch retinal vein occlusion; anterior ischemic optic neuropathy; non-retinopathy diabetic retinal dysfunction; retinoschisis; retinitis pigmentosa; glaucoma; Usher syndrome, cone-rod dystrophy; Stargardt disease (fundus flavimaculatus); inherited macular degeneration; chorioretinal degeneration; Leber congenital amaurosis; congenital stationary night blindness; choroideremia; Bardet-Biedl syndrome; macular telangiectasia; Leber's hereditary optic neuropathy; retinopathy of prematurity; and disorders of color vision, including achromatopsia, protanopia, deuteranopia, and tritanopia.

In some cases, a subject that can be administered a subject composition is receiving, receives, or receives after an administration a secondary therapy. A secondary therapy can comprise any therapy for ocular use. In some cases, a secondary therapy comprises nutritional therapy, vitamins, laser treatment, such as laser photocoagulation, photodynamic therapy, Visudyne, anti-VEGF therapy, eye-wear, eye drops, numbing agents, Orthoptic vision therapy, Behavioral/perceptual vision therapy, and the like. In some aspects, any of the previously described biologics can be considered a secondary therapy.

The present disclosure provides a method of delivering a transgene to a retinal cell in an individual, the method comprising administering to the individual a subject AAV virion as described above. Delivering a transgene to a retinal cell can provide for treatment of a retinal disease. The retinal cell can be a photoreceptor, a retinal ganglion cell, a Müller cell, a bipolar cell, an amacrine cell, a horizontal cell, or a retinal pigmented epithelial cell. In some cases, the retinal cell is a photoreceptor cell, e.g., a rod or cone cell.

The present disclosure provides a method of treating a retinal disease, the method comprising administering to an individual in need thereof an effective amount of a subject AAV virion as previously described. A subject AAV virion can be administered via intraocular injection, by intravitreal injection, or by any other convenient mode or route of administration. Other convenient modes or routes of administration include, e.g., intravenous, intranasal, etc. When administered via intravitreal injection, a subject virion is able to move through the vitreous and traverse the internal limiting membrane (also referred to herein as an inner limiting membrane, or “ILM”; a thin, transparent acellular membrane on the surface of the retina forming the boundary between the retina and the vitreous body, formed by astrocytes and the end feet of Mueller cells), and/or moves through the layers of the retina more efficiently, compared to the capability of an AAV virion comprising the corresponding parental AAV capsid protein.

The disclosure herein also provides a method of treating a retinal disease, the method comprising administering to an individual in need thereof an effective amount of a modified AAV virion comprising a transgene of interest as described above and disclosed herein. One of ordinary skill in the art would be readily able to determine an effective amount of a subject virion and that the disease had been treated by testing for a change in one or more functional or anatomical parameters, e.g. visual acuity, visual field, electrophysiological responsiveness to light and dark, color vision, contrast sensitivity, anatomy, retinal health and vasculature, ocular motility, fixation preference, and stability.

Nonlimiting methods for assessing retinal function and changes thereof include assessing visual acuity (e.g. best-corrected visual acuity [BCVA], ambulation, navigation, object detection and discrimination), assessing visual field (e.g. static and kinetic visual field perimetry), performing a clinical examination (e.g. slit lamp examination of the anterior and posterior segments of the eye), assessing electrophysiological responsiveness to all wavelengths of light and dark (e.g. all forms of electroretinography (ERG) [full-field, multifocal and pattern], all forms of visual evoked potential (VEP), electrooculography (EOG), color vision, dark adaptation and/or contrast sensitivity). Nonlimiting methods for assessing anatomy and retinal health and changes thereof include Optical Conherence Tomography (OCT), fundus photography, adaptive optics scanning laser ophthalmoscopy (AO-SLO), fluorescence and/or autofluorescence; measuring ocular motility and eye movements (e.g. nystagmus, fixation preference, and stability), measuring reported outcomes (patient-reported changes in visual and non-visually-guided behaviors and activities, patient-reported outcomes [PRO], questionnaire-based assessments of quality-of-life, daily activities and measures of neurological function (e.g. functional Magnetic Resonance Imaging (MRI)).

In some embodiments, an effective amount of the subject rAAV virion results in a decrease in the rate of loss of retinal function, anatomical integrity, or retinal health, e.g. a 2-fold, 3-fold, 4-fold, or 5-fold or more decrease in the rate of loss and hence progression of disease, for example, a 10-fold decrease or more in the rate of loss and hence progression of disease. In some embodiments, the effective amount of the subject rAAV virion results in a gain in visual function, retinal function, an improvement in retinal anatomy or health, and/or an improvement in ocular motility and/or improvement in neurological function, e.g. a 2-fold, 3-fold, 4-fold or 5-fold improvement or more in retinal function, retinal anatomy or health, and/or improvement in ocular motility, e.g. a 10-fold improvement or more in retinal function, retinal anatomy or health, and/or improvement in ocular motility. As will be readily appreciated by the ordinarily skilled artisan, the dose required to achieve the desired treatment effect will typically be in the range of 1×10⁸ to about 1×10¹⁵ recombinant virions, typically referred to by the ordinarily skilled artisan as 1×10⁸ to about 1×10¹⁵ “vector genomes”.

In some aspects, compositions provided herein, such as pharmaceutical compositions are administered to a subject in need thereof. In some cases, an administration comprises delivering a dosage of an AAV vector of about vector 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, 1.5×10¹⁵ vg. For example, for in vivo injection, i.e., injection directly into the eye, a therapeutically effective dose can be on the order of from about 10⁶ to about 10¹⁵ of subject AAV virions, e.g., from about 10⁸ to 10¹² engineered AAV virions. For in vitro transduction, an effective amount of engineered AAV virions to be delivered to cells will be on the order of from about 10⁸ to about 10¹³ of the engineered AAV virions. Other effective dosages can be readily established by one of ordinary skill in the art through routine trials establishing dose response curves.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or super ocular injection.

In some aspects, a subject can be screened via genetic testing for a mutation before, during, and/or after administration of a pharmaceutical composition provided herein. Relevant genes that can be screened for mutations comprise: RPE65, CRB1, AIPL1, CM, or RPGRIP.

Also provided are kits comprising any of the compositions provided herein. Provided is also a container that comprises: a) a subject modified adeno-associated virus (AAV) capsid; b) a subject vector; or c) a subject engineered virion. In an aspect, the container is a vial, syringe, or needle. In some cases, the container is configured for ocular delivery.

Kits may comprise a suitably aliquoted composition. The components of the kits may be packaged either in aqueous media or in lyophilized form. The container means of the kits will generally include at least one vial, test tube, flask, bottle, syringe, or another container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

In some instances, a packaged product comprising a composition described herein can be properly labeled. In some instances, the pharmaceutical composition described herein can be manufactured according to good manufacturing practice (cGMP) and labeling regulations. In some cases, a pharmaceutical composition disclosed herein can be aseptic.

### EXAMPLES

### Example 1: Design of Modified AAV Capsid

A decanoyl-L-peptidyl fragment (10 amino acid residues) was partially randomized for initial analysis. The starting amino acid sequence of the initial peptide fragment is provided as formula 1: X0-X1-X2-X1-X3-X1-X1-X4 (SEQ ID NO: 108), wherein X0 is Valine (V), Isoleucine (I), Leucine (L), Phenylalanine (F), Tryptophan (W), Tyrosine (Y) or Methionine (M), wherein X1 is Alanine (A), Asparagine (N), Glutamine (Q), Serine (S), Threonine (T), Glutamic Acid (E), Aspartic Acid (D), Lysine (K), Arginine (R), or Histidine (H), wherein X2 is V, I, L, or M, wherein X3 is E, S, or Q, wherein X4 is K, R, E, or A, and wherein X5 is Proline (P) or R.

The modified peptide is partially inserted into loop 3 and/or loop 4 of an AAV2 (or other suitable AAV serotypes as provided herein) viral protein (VP1) and analyzed using public structural modeling system. Conformations having a similar phenotypic and/or structural appearance as compared to the wild type loop structures of an unmodified AAV2 capsid were selected for biological evaluation. Table 2: provides exemplary AAV2 Rep and modified AAV2 capsid regions.

TABLE 2
Exemplary exogenous polypeptide sequences that can
be inserted into AAV capsids.

| SEQ ID | | Modified Capsid | Exogenous |
|---|---|---|---|
| NO | Clone No. | Region of AAV2 | Polypeptide Sequence |
| 6 | V466 | 453 | LALGETTRPA |
| 7 | V467 | 587 | LALGETTKPA |
| 8 | V468 | 452 | ALGETTKP |
| 9 | V471 | 452 | ALGETTKP |
| 10 | V471 | 587 | LALGETTKPA |
| 11 | AMI051 | 587 | LKLGQTTKPK |
| 12 | AMI052 | 587 | LALGQTTKPK |
| 13 | AMI053 | 587 | LKLGQTTKPA |
| 14 | AMI054 | 587 | LALGQTTKPA |
| 15 | AMI097 | 453 | LALGQTTKPA |
| 16 | AMI098 | 587 | LALGQTTEPA |
| 17 | AMI099 | 588 | LALGQTTKPA |
| 18 | AMI100 | 585 | LALGQTTKPA |
| 19 | AMI101 | 587 | VALGQTTKPA |
| 20 | AMI102 | 586 | LALGESTARG |
| 21 | AMI103 | 586 | LALGETSKRA |
| 22 | AMI104 | 586 | LALGQSTKPA |
| 23 | AMI105 | 452 | LALGQTTKPA |
| 24 | AMI106 | 453 | LALGQTTKPA |
| 25 | AMI106 | 587 | LALGQTTKPA |
| 26 | AMI107 | 587 | LALGQTTKPALALGQTTKPA |
| 27 | AMI110 | 587 | VKLGQTTKPA |

| SEQ ID | | |
|---|---|---|
| NO | Clone | Modified AAV2 Capsid Sequence |
| 28 | V466 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGETTRPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 29 | V467 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGETTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 30 | V468 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSALGETTKPGTTTQSRLQFSQAGASDIR |
| | | DQSRNWLPGPCYRQQRVSKTSADNNNSEYSWTGATKYHL |
| | | NGRDSLVNPGPAMASHKDDEEKFFPQSGVLIFGKQGSEKT |
| | | NVDIEKVMITDEEEIRTTNPVATEQYGSVSTNLQRGNRQAA |
| | | TADVNTQGVLPGMVWQDRDVYLQGPIWAKIPHTDGHFHP |
| | | SPLMGGFGLKHPPPQILIKNTPVPANPSTTFSAAKFASFITQY |
| | | STGQVSVEIEWELQKENSKRWNPEIQYTSNYNKSVNVDFT |
| | | VDTNGVYSEPRPIGTRYLTRNL |
| 31 | V471ª | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSALGETTKPGTTTQSRLQFSQAGASDIR |
| | | DQSRNWLPGPCYRQQRVSKTSADNNNSEYSWTGATKYHL |
| | | NGRDSLVNPGPAMASHKDDEEKFFPQSGVLIFGKQGSEKT |
| | | NVDIEKVMITDEEEIRTTNPVATEQYGSVSTNLQRGNLALG |
| | | ETTKPARQAATADVNTQGVLPGMVWQDRDVYLQGPIWA |
| | | KIPHTDGHFHPSPLMGGFGLKHPPPQILIKNTPVPANPSTTFS |
| | | AAKFASFITQYSTGQVSVEIEWELQKENSKRWNPEIQYTSN |
| | | YNKSVNVDFTVDTNGVYSEPRPIGTRYLTRNL |
| 32 | AMI051 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLKLGQTTKPKR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 33 | AMI052 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPKR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 34 | AMI053 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLKLGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 35 | AMI054 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 36 | AMI097 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGQTTKPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 37 | AMI098 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTEPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 38 | AMI099 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNRLALGQTTKPA |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 39 | AMI100 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRLALGQTTKPAGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 40 | AMI101 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNVALGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 41 | AMI102 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGESTARGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 42 | AMI103 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGETSKRANR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 43 | AMI104 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGQSTKPANR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 44 | AMI105 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSLALGQTTKPAGTTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 45 | AMI106 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGQTTKPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNL |
| | | ALGQTTKPARQAATADVNTQGVLPGMVWQDRDVYLQGPI |
| | | WAKIPHTDGHFHPSPLMGGFGLKHPPPQILIKNTPVPANPST |
| | | TFSAAKFASFITQYSTGQVSVEIEWELQKENSKRWNPEIQY |
| | | TSNYNKSVNVDFTVDTNGVYSEPRPIGTRYLTRNL |
| 46 | AMI107 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPAL |
| | | ALGQTTKPARQAATADVNTQGVLPGMVWQDRDVYLQGPI |
| | | WAKIPHTDGHFHPSPLMGGFGLKHPPPQILIKNTPVPANPST |
| | | TFSAAKFASFITQYSTGQVSVEIEWELQKENSKRWNPEIQY |
| | | TSNYNKSVNVDFTVDTNGVYSEPRPIGTRYLTRNL |
| 47 | AMI110 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNVAKGQTTKPA |
| | | RQAATADVNTQGVLPGMVWQDRDVYLQGPIWAKIPHTD |
| | | GHFHPSPLMGGFGLKHPPPQILIKNTPVPANPSTTFSAAKFA |
| | | SFITQYSTGQVSVEIEWELQKENSKRWNPEIQYTSNYNKSV |
| | | NVDFTVDTNGVYSEPRPIGTRYLTRNL |

Exemplary insertion sites are shown for AAV2 but comparable locations of other AAV serotypes are also contemplated.
Clone V471 has double insertion of peptides at 452 and 587 positions for SEQ ID NO: 26.

### Example 2: Generation of Modified AAV Capsids

### Construction of pFB Shuttle Plasmids Carrying AAV2 Rep and Modified AAV2 Capsid Gene

PCR was utilized to insert the desired mutant peptide into the AAV2 capsid gene of the V449 plasmid backbone. Briefly, plasmid V449-pFB-inCap2-inRep-kozak-hr2 was cut with restriction enzymes BsiWI and XbaI (New England Biolabs, Ipswich, MA) to isolate the backbone fragment of 9113 bp. A 5′ PCR fragment and a 3′-PCR fragment which contain the DNA sequences of the desired mutant peptide was amplified respectively using V449-pFB-inCap2-inRep-kozak-hr2 as template and then joined together to form a single PCR fragment by a second PCR. The joined PCR fragments were respectively cloned into the BsiWI and XbaI sites of V449-pFB-inCap2-inRep-kozak-hr2 using the NEBuilder HiFi DNA Assembly kit (New England Biolabs) to create the desired clones. However, clone V471-pFB-inCap2-452_587RtoK-inRep-kozak-hr2 was created using V467-pFB-inCap2-587RtoK-inRep-kozak-hr2 as template for PCR reactions and as backbone for HiFi assembly. The resulted clones were verified with restriction digestion, and the mutation sites were confirmed by DNA sequencing analysis. The clone numbers and PCR primers are provided in Table 3.

TABLE 3
List of clone number and corresponding PCR primers used to generate
the PCR fragments during cloning. Note: a. A035 has the same DNA
sequence as 1874; b. A038 has the same DNA sequence as 1871.

| | 5′ PCR Fragment | | 3′ PCR Fragment | | Joined PCR Fragment | |
|---|---|---|---|---|---|---|
| Clone No. | F-primer | R-primer | F-primer | R-primer | F-primer | R-primer |
| V466 | 1874 | 6334 | 1871 | 6333 | 1874 | 1871 |
| V467 | 1874 | 6336 | 1871 | 6335 | 1874 | 1871 |
| V468 | 1874 | 6351 | 1871 | 6352 | 1874 | 1871 |
| V471 | 1874 | 6351 | 1871 | 6352 | 1874 | 1871 |
| AMI051 | A035 | A036 | A038 | A037 | A035 | A038 |
| AMI052 | A035 | A039 | A038 | A040 | A035 | A038 |
| AMI053 | A035 | A041 | A038 | A042 | A035 | A038 |
| AMI054 | A035 | A043 | A038 | A044 | A035 | A038 |
| AMI055 | A035 | A045 | A038 | A046 | A035 | A038 |
| AMI056 | A035 | A047 | A038 | A048 | A035 | A038 |
| AMI057 | A035 | A049 | A038 | A050 | A035 | A038 |
| AMI097 | A035 | A280 | A038 | A279 | A035 | A038 |
| AMI098 | A035 | A284 | A038 | A283 | A035 | A038 |
| AMI099 | A035 | A286 | A038 | A285 | A035 | A038 |
| AMI100 | A035 | A290 | A038 | A289 | A035 | A038 |
| AMI101 | A035 | A292 | A038 | A291 | A035 | A038 |
| AMI102 | A035 | A298 | A038 | A297 | A035 | A038 |
| AMI103 | A035 | A300 | A038 | A299 | A035 | A038 |
| AMI104 | A035 | A302 | A038 | A301 | A035 | A038 |

Sequences of primers used for PCR reactions and DNA sequencing analysis are provided in Table 4.

TABLE 4
DNA sequence of primers used for PCR reactions used to
generate modified AAV capsid.

| SEQ | | |
|---|---|---|
| ID | Primer | |
| NO | ID | DNA sequence (5′ to 3′) |
| 48 | 1871 | CGAGACTGCAGGCTCTAGATTACAG |
| 49 | 1874 | GGAGTACCAGCTCCCGTACGTCCTC |
| 50 | 6333 | CTAGCACTCGGCGAAACAACAAGACCTGCTACCACCACGCAGTCAAG |
| | | GC |
| 51 | 6334 | AGCAGGTCTTGTTGTTTCGCCGAGTGCTAGTCCACTTGGAGTGTTTGTT |
| 52 | 6335 | CTAGCACTCGGCGAAACAACAAAGCCTGCTAGGCAAGCAGCTACCGC |
| | | AG |
| 53 | 6336 | AGCAGGCTTTGTTGTTTCGCCGAGTGCTAGGTTGCCTCTCTGGAGGTT |
| | | G |
| 54 | 6351 | AGGCTTTGTTGTTTCGCCGAGTGCACTTGGAGTGTTTGTTCTGC |
| 55 | 6352 | GCACTCGGCGAAACAACAAAGCCTGGAACCACCACGCAGTCAAG |
| 56 | A035ª | GGAGTACCAGCTCCCGTACGTCCTC |
| 57 | A036 | CTTAGGCTTTGTTGTCTGGCCGAGCTTTAGGTTGCCTCTCTGGAGGTTG |
| 58 | A037 | CTAAAGCTCGGCCAGACAACAAAGCCTAAGAGGCAAGCAGCTACCGC |
| | | AG |
| 59 | A038^{b} | CGAGACTGCAGGCTCTAGATTACAG |
| 60 | A039 | CTTAGGCTTTGTTGTCTGGCCGAGTGCTAGGTTGCCTCTCTGGAGGTTG |
| 61 | A040 | CTAGCACTCGGCCAGACAACAAAGCCTAAGAGGCAAGCAGCTACCGC |
| | | AG |
| 62 | A041 | TGCAGGCTTTGTTGTCTGGCCGAGCTTTAGGTTGCCTCTCTGGAGGTTG |
| 63 | A042 | CTAAAGCTCGGCCAGACAACAAAGCCTGCAAGGCAAGCAGCTACCGC |
| | | AG |
| 64 | A043 | TGCAGGCTTTGTTGTCTGGCCGAGTGCTAGGTTGCCTCTCTGGAGGTT |
| | | G |
| 65 | A044 | CTAGCACTCGGCCAGACAACAAAGCCTGCAAGGCAAGCAGCTACCGC |
| | | AG |
| 66 | A045 | TAGCTGCTTGTGCGTTGCCTGCCTGGAGGTTGGTAGATACAGAAC |
| 67 | A046 | ACCTCCAGGCAGGCAACGCACAAGCAGCTACCGCAGATGTCAACA |
| 68 | A047 | TAGCTGCTTGCCTGTTGCCTGCCTGGAGGTTGGTAGATACAGAAC |
| 69 | A048 | ACCTCCAGGCAGGCAACAGGCAAGCAGCTACCGCAGATGTCAACA |
| 70 | A049 | TAGCTGCTTGTGCGTTGCCTCTCTGGAGGTTGGTAGATACAGAAC |
| 71 | A050 | ACCTCCAGAGAGGCAACGCACAAGCAGCTACCGCAGATGTCAACA |
| 72 | A279 | CTAGCACTCGGCCAGACAACAAAGCCTGCAACCACCACGCAGTCAAG |
| | | GC |
| 73 | A280 | TGCAGGCTTTGTTGTCTGGCCGAGTGCTAGTCCACTTGGAGTGTTTGTT |
| 74 | A281 | CTAGCACTCGGCCAGACAACAAAGCCTGCAGGAACCACCACGCAGTC |
| | | AA |
| 75 | A282 | TGCAGGCTTTGTTGTCTGGCCGAGTGCTAGACTTGGAGTGTTTGTTCTG |
| 76 | A283 | CTAGCACTCGGCCAGACAACAGAGCCTGCAAGGCAAGCAGCTACCGC |
| | | AG |
| 77 | A284 | TGCAGGCTCTGTTGTCTGGCCGAGTGCTAGGTTGCCTCTCTGGAGGTT |
| | | G |
| 78 | A285 | CTAGCACTCGGCCAGACAACAAAGCCTGCACAAGCAGCTACCGCAGA |
| | | TG |
| 79 | A286 | TGCAGGCTTTGTTGTCTGGCCGAGTGCTAGCCTGTTGCCTCTCTGGAG |
| | | G |
| 80 | A289 | CTAGCACTCGGCCAGACAACAAAGCCTGCAGGCAACAGGCAAGCAGC |
| | | TA |
| 81 | A290 | TGCAGGCTTTGTTGTCTGGCCGAGTGCTAGTCTCTGGAGGTTGGTAGA |
| | | T |
| 82 | A291 | GTGGCACTCGGCCAGACAACAAAGCCTGCAAGGCAAGCAGCTACCGC |
| | | AG |
| 83 | A292 | TGCAGGCTTTGTTGTCTGGCCGAGTGCCACGTTGCCTCTCTGGAGGTT |
| | | G |
| 84 | A293 | CTAGCACTCGGCCAGACAACAAAGCCTGCAGGAACCACCACGCAGTC |
| | | AA |
| 85 | A294 | TGCAGGCTTTGTTGTCTGGCCGAGTGCTAGACTTGGAGTGTTTGTTCTG |
| 86 | A297 | CTGGCACTCGGCGAGTCGACAGCGCGGGGCAACAGGCAAGCAGCTAC |
| | | CG |
| 87 | A298 | GCCCCGCGCTGTCGACTCGCCGAGTGCCAGGCCTCTCTGGAGGTTGGT |
| | | A |
| 88 | A299 | CTGGCACTCGGCGAGACAAGCAAGCGGGCAAACAGGCAAGCAGCTAC |
| | | CG |
| 89 | A300 | TGCCCGCTTGCTTGTCTCGCCGAGTGCCAGGCCTCTCTGGAGGTTGGT |
| | | A |
| 90 | A301 | CTAGCACTCGGCCAGAGCACAAAGCCTGCAAACAGGCAAGCAGCTAC |
| | | CG |
| 91 | A302 | TGCAGGCTTTGTGCTCTGGCCGAGTGCTAGGCCTCTCTGGAGGTTGGT |
| | | A |

### Generation of Recombinant Baculoviruses

Recombinant baculoviruses were generated using the Bac-to-Bac Baculovirus Expression System according to the manufacturer's instructions (Invitrogen, Carlsbad, CA). Briefly, the pFB shuttle plasmids containing the target genes as previously described were each diluted into 1 ng/1 μL in TE buffer, and 2 ng of each DNA was mixed with 20 μL of Δcath-DH10Bac competent bacteria containing a bacmid DNA molecule with the cathepsin gene deleted (Virovek, Hayward, CA) and incubated on ice for 30 min followed by heat-shock at 42° C. for 30 seconds. After incubation on ice for 2 min, the bacteria were cultured at 37° C. for 4 hours to recover and then plated on agar plates containing 50 μg/mL of kanamycin, 7 μg/mL of gentamycin, 10 μg/mL of tetracycline, 40 μg/mL of IPTG, and 100 μg/mL of X-gal. After 48 hours of incubation at 37° C., white colonies containing the recombinant bacmid DNAs were picked and miniprep bacmid DNAs purified under sterile condition. About 5 μg of each bacmid DNA and 10 μL of GeneJet Reagent (SignaGen Laboratories, Fredrick, MD) were respectively diluted in 100 uL ESFAF media (Expression Systems, Davis, CA) and then mixed together for about 30 min to form the transfection mixture. Sf9 cells were plated in the 6-well plate at 1.5e+6 cells/well in 2 mL ESFAF media at 28° C. for about 30 min. After removing the old media from the Sf9 cells, each transfection mixture was diluted in 800 μL ESFAF media and then added to the Sf9 cells. After incubation at 28° C. overnight, each well with Sf9 cells were added with additional 1 mL ESFAF media. After a total incubation time of 4 days, recombinant baculoviruses were collected without the cells and amplified at 1:200 ratio to generate sufficient quantity of recombinant baculoviruses ready for use in the AAV production process.

Sf9 cell cultures were maintained in ESF AF media (Expression Systems) containing 100 units/mL penicillin and 100 ug/mL streptomycin (Thermo Fisher Scientific, Pleasanton, CA) in Corning bottle with gentle shaking at 150 rpm and 28° C. Once cells grow to −1e+7 cells/mL, they were split 1:4 in fresh media into a new bottle and continuously cultured for maintenance purpose.

### Example 3: AAV Production and Purification

The recombinant baculoviruses respectively carrying the AAV2 Rep gene and the mutant AAV2 capsid gene, and the CMV-GFP, were used to co-infect Sf9 cells for AAV production. Briefly, 10 moi of rBV carrying the Rep and Cap genes and 5 moi of rBV carrying the CMV-GFP were used to co-infect the Sf9 cell line at density of ˜5e+6 cells/mL with 50% fresh ESFAF media for 3 days at 28° C. with shaking speed of 180 rpm in a shaker incubator. At the end of infection, cell pellets were collected by centrifugation at 3,000 rpm for 10 min. The cells were lysed in Sf9 lysis buffer containing Tris-HCl, pH8.0, 2 mM MgCl2, 1% sarkosyl, 1% Triton X-100, and 125 units/mL benzonase with vigorous vortex followed by shaking at 350 rpm, 37° C. for 1 hour. At the end of shaking, salt concentration was adjusted to 500 mM and the lysates were cleared by centrifugation at 8,000 rpm for min at 4° C. The cleared lysates were transferred to ultraclear centrifuge tubes for SW28 swing bucket rotor which contain 5 mL 1.50 g/cc and 10 mL 1.30 g/cc cesium chloride solutions. After centrifugation at 28,000 rpm, 15° C. for ˜18 hours, the AAV bands were collected with syringes and transferred to ultraclear centrifuge tubes for a 70 ti centrifuge rotor. The centrifuge tubes were filled with 1.38 g/cc cesium chloride solution and heat-sealed. The AAV samples were subjected to a second round of ultracentrifugation at 65,000 rpm, 15° C. for ˜18 hours and AAV bands were collected with syringes. The purified AAV samples were buffer-exchanged into PBS buffer containing 0.001% Pluronic F-68 and filter-sterilized with 0.22 μm syringe filters. The sterilized AAV samples were stored at 4° C. within a month and then transferred to −80° C. for long term storage. AAV titer was determined with real-time PCR method using the QuantStudio 7 Flex Real-Time PCR System (Invitrogen). In some aspects, the AAV titer can be determined by TCID50 (median tissue culture infectious dose) AAV vector infection titer assay. The TCID50 AAV titer assay can be measured first by culturing cells to be transduced into complete media comprising DMEM supplanted with 10% FBS and 1% penicillin/streptomycin solution. The cells can be cultured in 96-well plates and subsequently transduced by any one of the modified AAV described herein. The transduction can include diluting adenovirus to make a final concentration of 3.2e+8 adenovirus particles per milliliter. The modified AAV vector can be diluted by serial dilution for the titer assay. The cells can then be transduced by the different serially diluted modified AAV vector. DNA can be extracted from the transduced cells. The extracted DNA can then be assayed with Taqman probes under Taqman thermocycling condition to obtain amplification signal. The exponential phase of the amplification signal can correspond to the concentration obtained from the serial dilution of the modified AAV vector.

### Gel Electrophoresis

To visualize AAV capsid proteins, SDS-PAGE electrophoresis was performed. AAV samples were mixed with SDS-gel loading buffer, heat at 95° C. for 5 min and 1e+11vg/lane of AAV vectors were loaded and electrophoresed at 100V until the loading dye reached the gel bottom. AAV capsid proteins were stained with SimplyBlue SafeStain Kit (Thermo Fisher Scientific). Gel images were recorded with BIO-RAD Molecular Imager Gel-Doc XR+ (Bio-Rad, Hercules, CA). To visualize AAV genomes, agarose gel electrophoresis was performed. AAV samples were diluted in PBS buffer containing 0.1% SDS and heated at 80° C. for 10 min. After turned off the heater to slowly cool down to room temperature, 2e+11 vg/lane of AAV vectors were loaded on 1% agarose gel in TAE buffer containing SYBR Safe DNA gel Stain (Invitrogen, Carlsbad, CA) and electrophoresed at 100V for 1 hour. Images were recoded with the BIO-RAD Molecular Imager.

### Cell Culture and In Vitro Transduction ARPE19 and HEK293

ARPE-19 (ATCC CRL2302) is a suitable model for RPE cells because it expresses typical RPE markers such as cellular retinaldehyde-binding protein (CRALBP) and RPE-specific 65 kDa protein (RPE65). Cells were maintained in Dulbecco's modified Eagle's medium/F-12 nutrient medium (DMEM/F-12; Gibco BRL, Carlsbad, CA, USA) supplemented with 10% heat-inactivated fetal bovine serum (Gibco BRL) and 1% penicillin-streptomycin. Cells were kept in an incubator at 37° C. and 5% CO2 in a humidified atmosphere, and passaged with 0.25% Trypsin-EDTA (Life Technologies, Carlsbad, CA, USA) every 3-4 days. RPE cells within the first 10 passages were selected and placed into appropriate culture plates for the experiments.

HEK293 cells were obtained from ATCC (CRL-1573; Manassas, VA, USA) and cultured in high glucose Dulbecco's modified Eagle's medium (DMEM; Gibco BRL, Carlsbad, CA, USA). Media was supplemented with 10% fetal bovine serum (Gibco BRL) and 1% penicillin-streptomycin. Cells were kept in an incubator at 37° C. and 5% CO2 in a humidified atmosphere, and passaged with 0.25% Trypsin-EDTA (Life Technologies, Carlsbad, CA, USA) every 3-4 days.

### Example 4: In Vitro Transduction Assay of AAV2 Capsid Variants

ARPE-19 cells or HEK293 cells were seeded at 60% confluency in 12-well plates and grown to sub confluency. Cells were fed 2-4 h prior infection, media changed to 2% FBS and cells were exposed to wild-type or capsid modified AAV2-CMV-GFP in 1 ml for 1 h. Afterwards, complete medium was added, and incubation continued from 3 to 7 days. All infections were performed with the same number of vg (2.0e+13), which resulted in an estimate MOI of 1.0e+5 vg/cell. The expression of GFP was analyzed in an inverted fluorescence microscope (Olympus, Center Valley, PA, USA) equipped with a monochrome DP80 camera. All images were captured at the same non-saturated exposure time using digiCAMControl.

Transduction of ARPE-19 cells, HEK293 cells, or any other cell types with any one of the modified AAV vector can include utilizing the following materials and equipment: AAV2.N54. VEGF-Trap (AMI054-AMI120-scCMV-Aflibercept-GCRS), also known as AVMX-110, was purified and buffer exchanged to formulation buffer (concentration at 9.23e+12 vg/mL and was stored at −80° C.); Human Embryonic kidney (HEK293, ATCC, Cat #CRL-1573) cells were thawed and passage number was documented routinely; EMEM medium (ATCC, Cat #30-2003) with 10% FBS (ATCC, Cat #30-2020) was used to culture and transduce cells. Store the medium at 4° C.; Trypsin-EDTA (0.25%), phenol red (Thermofisher Scientific, Cat #25200056); 1×PBS (Phosphate buffered saline): Dilute 10×PBS (Fisher Scientific, Hyclone, Cat #SH3025802) with culture grade sterile water (Fisher Scientific, Hyclone, Cat #SH30529LS). Store at 4° C.; 6-well culture plate (Greiner bio-one/Cellstar, Cat #657 160); Biosafety cabinet (Labconco Purifier Class II); Incubator set at 37° C. and 5% CO2 (Therma forma, Series II water jacketed CO2 incubator); and V226-AMI067 (positive control, V226-pFB-inCap2-7m8-AMI067-scCMV-Aflibercept-GC). The transduction procedure can include: culturing HEK293 cells (or any other cell type) in T-75 flask with EMEM medium supplemented with 10% FBS keeping record of passage number and cell viability; taking the flask out of incubator and removing the old medium and rinse the flask with 10 mL of 1×PBS; discarding 1x PBS and add 0.5 mL of trypsin solution and let the flask rest for 1-2 minutes; adding 10 times v/v (5 mL) of medium containing serum to neutralize trypsin and allow cells being dissociated from the culture flask; transferring cell suspension into a 15 mL tube and centrifuge at 300 g for 3 minutes; adding 1 mL of EMEM medium to resuspend the cells. Count cells and adjust the cell number to 1.0e+06/mL by adding medium; adding 1 mL into each well of 6-well plate; putting the plate back into incubator for 24 hours to let the cells adhere to the bottom of wells; after 24 hours preparing AAV2.N54.VEGF-Trap and V226-AMI067 (positive control) at MOI 100,000 to total amount of viral particles at 1.0e+11 (=1.0e+06*100,000)/mL in fresh medium; adding 1 mL of AAV2.N54.VEGF-Trap, or positive control, or fresh medium (negative control) to the wells, in duplicate; placing the 6-well plate back into the incubator; after another 24 hours replacing medium with 1 mL/well of fresh medium in all wells and place the plate back into the incubator; at day 4 after transduction collecting supernatant from each well of the plate and aliquot into 1.5 mL sterile Eppendorf tubes at 300-500 μL/tube according to the assay requirements; and quantifying VEGF-Trap concentration in each collected supernatant using the VEGF-Trap ELISA or storing the supernatants at −80° C. Table 5 illustrates an exemplary study of AAV infectivity showing that the modified AAV vector (modified AAV2.N54) exhibited increased infectivity compared to wild type AAV2.

TABLE 5
AAV2.N54 showed 3× higher infectivity than wild type AAV2
reference standard
Infectivity of AAV2 VP1 capsid with N54 peptide insertion and
AAV2 wildtype reference standard

| | AAV2.N54-Aflibercept | | AAV2-WT | |
|---|---|---|---|---|
| | Product | Product | In-house | ATCC |
| | lot | lot | AAV2 | AAV2 |
| AAV2 Attribute | 21-038 | 21-045 | Control | Reference |
| AAV Titer (vg/mL) | 3.15 × 10¹³ | 20.8 × 10¹³ | 2.0 × 10¹³ | 3.28 × 10¹³ |
| Infectivity ^{(TCID}₅₀^{/mL)} | 1.12 × 10¹² | 2.42 × 10¹² | 3.16 × 10¹² | 8.51 × 10¹¹ |
| I/V Ratio | 28 | 9 | 63 | 39 |

| | | |
|---|---|---|
| Average particles | 18.5 | 52 |
| per unit | | |

| | |
|---|---|
| Enhancement | 3-fold |

### Example 5: Modification of AAV2 Tropism to the Retina

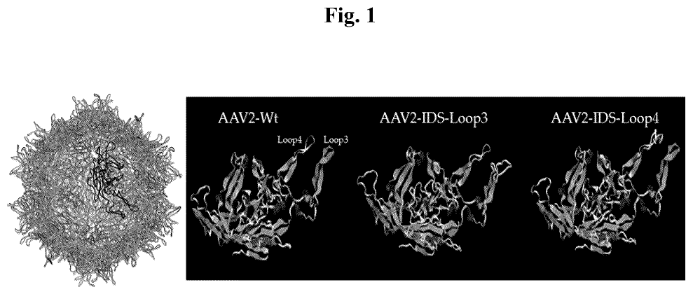

To study the capacity of AAV2 as a scaffold for peptide display and rAAV2 vector production, peptides with a length of 3-10 aa were displayed on AAV2. This range is well tolerated and can be sufficient for capsid retargeting. Regions in cap genes were selected for in-frame oligonucleotide insertion that promised display of the encoded peptides in a prominent location, i.e., Loop 3 and Loop 4, on the assembled particles. As shown in FIG. 1, the peptide insertions in rAAV2 is located at the top of either loop 3 or loop 4 which now extends further out than loop 3 or loop 4, unlike in the parental AAV2 (FIG. 1 right panel). A surface view of AAV2 structure indicates that the insertions occur on all 60 VP subunits.

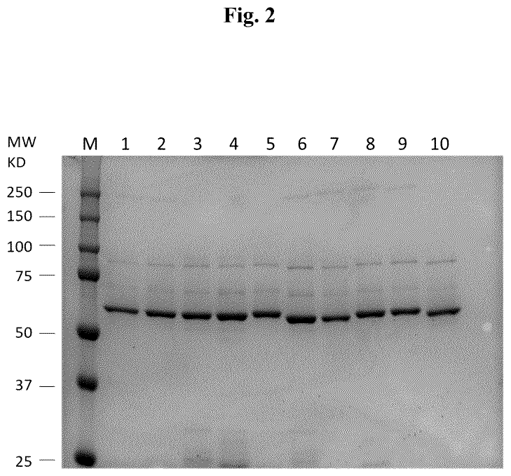

A series of AAV2 vectors with modified capsids were successfully produced and purified as shown in Table 6. SDS-PAGE indicates that after 2 rounds of cesium chloride density ultracentrifugation, the AAV2 vectors were more than 98% pure with only trace amounts of contaminating proteins as shown in FIG. 2. Table 7 illustrates expression of GFP (delivered into a cell via the AAV vector described herein) in HEK293 cell or ARPE19 cell, where the HEK293 cell or the ARPE19 cell was transduced with modified AAV vector described herein. Table 7 discloses SEQ ID NOS 110-113, 111, 114-126, 118, 117, and 127-128, respectively, in order of appearance.

TABLE 6
AAV-2 vector production (Vector prep quality, purity and
concentration)

| Clone No. | Lot No. | AAV Titer (vg/mL) |
|---|---|---|
| V466 | 19-480 | 3.68 × 10¹³ |
| V467 | 19-634 | 1.46 × 10¹³ |
| V468 | 19-544 | 2.33 × 10¹³ |
| V471^{a} | 19-679 | 1.14 × 10¹³ |
| AMI051 | A20-0606-01 | 1.42 × 10¹³ |
| AMI052 | A20-0606-02 | 1.63 × 10¹³ |
| AMI053 | A20-0606-03 | 1.91 × 10¹³ |
| AMI054 | A20-0606-04 | 2.04 × 10¹³ |
| AMI055^{b} | A20-0606-05 | 9.66 × 10¹² |
| AMI056^{c} | A20-0606-06 | 1.43 × 10¹³ |
| AMI057^{c} | A20-0606-07 | 1.66 × 10¹³ |
| AMI097 | 20-080 | 1.97 × 10¹³ |
| AMI098 | 20-081 | 2.06 × 10¹³ |
| AMI099 | 20-082 | 1.96 × 10¹³ |
| AMI100 | 20-095 | 1.75 × 10¹³ |
| AMI101 | 20-094 | 1.72 × 10¹³ |
| AMI102 | 20-083 | 1.19 × 10¹³ |
| AMI103 | 20-109 | 1.36 × 10¹³ |
| AMI104 | 20-110 | 1.95 × 10¹³ |
| AMI105 | 20-096 | 1.98 × 10¹³ |
| AMI106 | 20-106 | 1.71 × 10¹³ |
| AMI107 | 20-107 | 2.06 × 10¹³ |
| AMI110 | 20-098 | 1.59 × 10¹³ |

TABLE 7
Transduction of Human Cells Using Modified AAV-VP1 Packed
GFP.

| Construct | | HEK293 | ARPE19 |
|---|---|---|---|
| ID | Modified Capsid Region | Cell | Cell |
| Wt | No insertion | ++++ | ++++ |
| V466 | TPSG(453):LALGETTRPA:TTTQ | ++ | ++ |
| V467 | QRGN(587):LALGETTKPA:RQAA | ++++ | ++++ |
| V468 | NTPS(452) :- ALGETTKP:GTTT | ++ | ++ |
| V471^{a} | NTPS(452) :- ALGETTKP:GTTT; | ++ | ++ |
| | QRGN(587):LALGETTKPA:RQAA | | |
| AMI051 | QRGN(587):LKLGQTTKPK:RQAA | + | ND |
| AMI052 | QRGN(587):LALGQTTKPK:RQAA | | |
| AMI053 | QRGN(587):LKLGQTTKPA:RQAA | ++++ | ++++ |
| AMI054 | QRGN(587):LALGQTTKPA:RQAA | ++++ | ++++ |
| AMI055^{b} | R585_588A | + | ND |
| AMI056^{c} | R585A | ++ | ND |
| AMI057^{c} | R588A | ++ | ND |
| AMI097 | TPSG(453): LALGQTTKPA:TTTQ | ND | ND |
| AMI098 | QRGN(587):LALGQTTEPA:RQAA | ND | ND |
| AMI099 | RGNR(588):LALGQTTKPA:QAAT | ND | ND |
| AMI100 | NLQR(585):LALGQTTKPA:GNRQ | ND | ND |
| AMI101 | QRGN(587):VALGQTTKPA:RQAA | ND | ND |
| AMI102 | LQRG(586):LALGESTARG:NRQA | ND | ND |
| AMI103 | LQRG(586):LALGETSKRA:NRQA | ND | ND |
| AMI104 | LQRG(586):LALGQSTKPA:NRQA | ND | ND |
| AMI105 | NTPS(452):LALGQTTKPA:GTTT | ND | ND |
| AMI106 | TPSG(453):LALGQTTKPA:TTTQ | ND | ND |
| | QRGN(587):LALGQTTKPA:RQAA | | |
| AMI107 | QRGN(587):LALGQTTKPA | ND | ND |
| | LALGQTTKPA:RQAA | | |
| AMI110 | QRGN(587):VKLGQTTKPA:RQAA | ND | ND |

ND: not done but the experimentations are in progress

### Example 6: Effects of Modified AAV2 Capsid Variants on Transduction Efficiency in Human Cell Lines

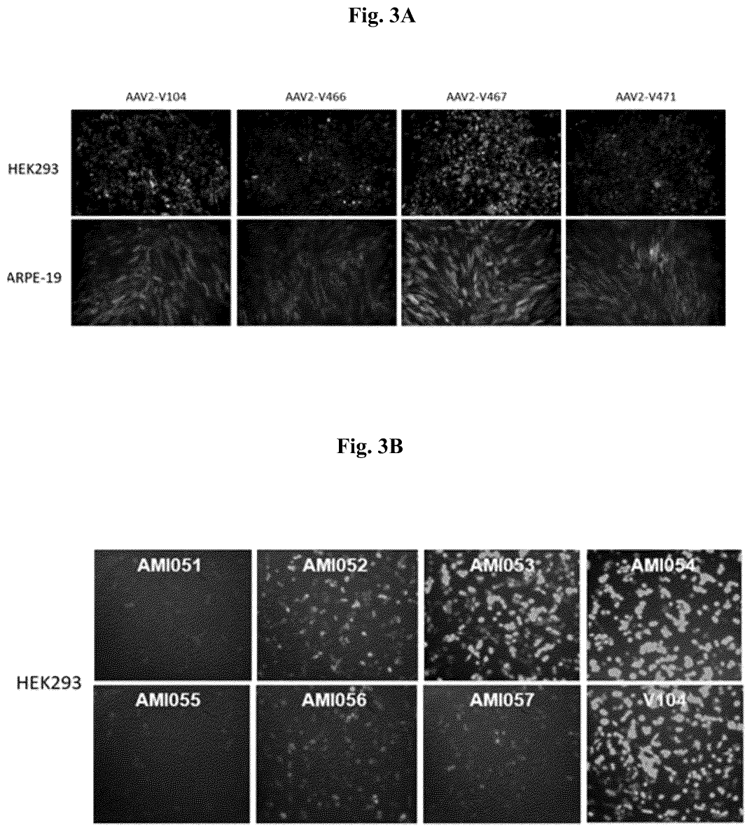

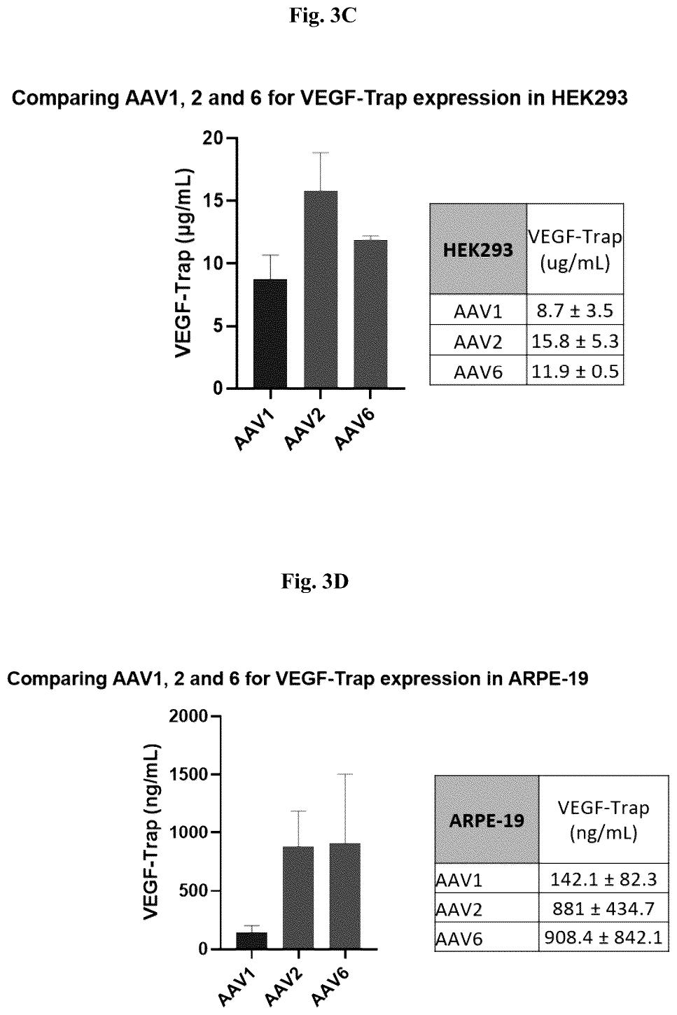

HEK 293 cells were utilized to determine the transduction efficiency or infectivity of AAV2 and ARPE-19, which is a spontaneously arising retinal pigment epithelial (RPE) cell line derived from the eye of a 19-year-old male. The transduction efficiency of all modified capsid variants was tested in both HEK293 and ARPE-19 cells. As shown in FIG. 3A, the expression levels of GFP in both HEK293 and ARPE-19 cells documented that the AAV2-V467 variant was the most efficient, followed by AAV2-V471 and AAV2-V466 variants. AAV2 variants with single mutation at the position of R585 or R588 of AAV2 or its combination failed to transduce cells (FIG. 3B). The insertion of other peptides in loop3 or loop4 of AAV2 capsid dramatically reduced the transgene expression.

VEGF-Trap can be measured with ELISA protocol described herein. In some aspects, the ELISA protocol utilizes the material and equipment such as: VEGF-trap; AAV-2-VEGF-trap 100 uL/vial, stored at −80° C. in PBS (the concentration is 4.56 mg/mL determined by commercial ELISA); VEGF: 100 μg/mL; antigen expressed in HEK293 cells and purified. (GeneScript, Cat #Z03073); Goat Anti-Human IgG Fc (Biotin) preadsorbed (Abcam, Cat #ab98618); HRP-Streptavidin conjugate (Abcam, Cat #ab7403); TMB substrate: 1-Step™ Ultra TMB-ELISA Substrate Solution (ThermoFisher, Cat #34028); 96-well microplate reader (Molecular Device: VERSAmax tunable Microplate reader); Coating buffer: 3.7 g sodium bicarbonate (NaHCO₃) and 0.64 g sodium carbonate (Na2CO3); 1 L of Milli Q water, pH 9.60, storage condition: RT (room temperature) for 1 month; 1x PBS (Phosphate buffered saline): 8.0 g sodium chloride, 1.3 g dibasic sodium phosphate, 0.2 g monobasic sodium phosphate, and 1.0-liter Milli-Q water, pH 7.4, storage condition: RT for 1 year, washing buffer (PBST): 1× Phosphate buffered saline, 0.1% Tween 20 (v/v), storage condition: RT for 30 days expiration from date of preparation; blocking buffer (BB): 1× phosphate buffered saline (PBS) with 0.1% Tween 20 (v/v) and with 1% Casein, storage condition: 4° C. for 30 days from date of preparation; Dilution buffer (DB): same as blocking buffer; stop Solution for TMB substrate (Abcam, Cat #ab171529); quality control sample: VEGF-trap standard (6 ng/mL) from the commercial Aflibercept ELISA kit; and 96-well microplate.

ELISA protocol can include the following procedure: diluting the VEGF (100 μg/mL) stock to 0.1 μg/mL with coating buffer (10 μL of VEGF stock to 10 mL of coating buffer); adding coating antigen to a 96-well microplate at 100 μL/well, cover and place the plate at 2-8° C. for approximately 12 hours or overnight; discarding the coating antigen and wash the plate thrice with 300 μL/well of PBS-T wash buffer; adding 300 μL/well of blocking buffer, cover and incubate the plate at 37±1° C. for 120 minutes; diluting VEGF-trap standard (4.56 mg/mL) with dilution buffer to 12.5 ng/mL as the following: 4.56 mg/mL was diluted 456-fold to 10 μg/mL, 10 μg/mL was diluted 10-fold to 1 μg/mL, and 1 μg/mL was diluted 10-fold to 100 ng/mL; adding 100 μL of 100 ng/mL solution in 700 μL of dilution buffer, this is the first standard point of 12.5 ng/mL; preparing the rest of VEGF-trap standard using 1:2 serial dilution scheme in duplicates; transferring the diluted standard samples, quality control samples and unknown samples to the plate, 100 μL per well in duplicates for each dilution; covering the plate and incubate the plate at 37±1° C. for 60 minutes; discarding the reactants in the plate and wash 6 times, 300 μL/well with wash buffer; diluting Goat Anti-Human IgG Fc (Biotin) preadsorbed at 1:40,000 with dilution buffer (0.0125 μg/mL) and adding 100 μL/well; covering and incubating the plate at 37±1° C. for 60 minutes; discarding the reaction mix and washing the plate 6 times, 300 μL/well with wash buffer; diluting streptavidin-HRP with dilution buffer at 1:40,000 and add 100 μL/well; covering and incubating the plate at 37±1° C. for 60 minutes; discarding the reactants in the plate and wash 6 times, 300 μL/well, with wash buffer; rinsing the plate with 1×PBS once to remove remaining Tween 20; adding TMB substrate at 100 μL/well, covering the plate and incubating at 37±1° C. for 15 minutes; stopping the reaction by adding stop solution 100 μL/well; and reading the plate at 450 nm wavelength filter with 600 nm as reference wavelength in a microplate reader.

FIG. 3C and FIG. 3D illustrate expression of VEGF-Trap gene mediated by VP1-AMI054 modified AAV1, AAV2 and AAV6: modified AAV1 comprising insertion of LALGQTTKPA exogenous polypeptide (SEQ ID NO: 14) after amino acid residue position 588 (asparagine, N); modified AAV2 comprising S588 insertion of LALGQTTKPA exogenous polypeptide (SEQ ID NO: 14) after amino acid residue position 588 (serine, S); and modified AAV6 comprising modified AAV2 comprising S588 insertion of LALGQTTKPA exogenous polypeptide (SEQ ID NO: 14) after amino acid residue position 588 (serine, S). FIG. 3C illustrates VEGF-Trap expression in HEK293 cell transduced by the modified AAV1, AAV2, or AAV6. FIG. 3D illustrates VEGF-Trap expression in ARPE-19 cell transduced by the modified AAV1, AAV2, or AAV6.

### Example 7: Murine Study

Animals are acclimated to the study environment for a minimum of 3 days. At the completion of the acclimation period, each animal is physically examined by a laboratory animal technician for determination of suitability for study participation. Examinations will include, but will not be limited to, the skin and external ears, eyes, abdomen, behavior, and general body condition. Animals determined to be in good health will be released to the study.

Animals are randomly assigned to study groups according to facility Standard Operating Procedures (SOPs). Animals are uniquely identified by corresponding cage card number, ear punch and number. Table 8 and Table 9 show the test parameters and diet for the murine study.

TABLE 8
Murine Testing System Parameters

| Species/Strain | Mouse (Mus Musculus)/C57BL/6 |
|---|---|
| Source | Charles River or Taconic Farms |
| Age Range at First Dosing | Approximately 8 weeks |
| Weight Range at First Dosing | 10-20 grams |
| Identification | Tail marking, ear punch, and cage card |
| Physical Examination Time | During acclimation |
| Caging | Innovive disposable mouse caging |
| Number per cage | 1-5 |
| Environmental Conditions | Photoperiod: 12 hrs. light/12 hrs. |
| | darkness |
| | Temperature: 68-79º F. |

TABLE 9
Murine Diet Parameters

| | | |
|---|---|---|
| Feed | Type | Lab Diet |
| | Name | 5P76 Prolab isopro irradiated |
| | Availability | ad libitum |
| | Analysis for Contaminants | Not routinely performed, No |
| | | contaminants expected |
| Water | Source | Durham City Water |
| | Availability | ad libitum via water bottles with |
| | | sipper tubes. |
| | Analysis for Contaminants | Every 6 months, no contaminants |
| | | found |

### Administration Scheme

On Day 0 prior to injection, mice were given buprenorphine 0.01-0.05 mg/kg SQ. A topical mydriatic (1.0% Tropicamide HCL, and 2.5% Phenylephrine HCL) was applied at least 15 minutes prior to the injection. Animals were tranquilized for the intravitreal injections and one drop of 0.5% proparacaine HCL was applied to both eyes. Alternatively, mice can be anesthetized with inhaled isoflurane. The conjunctiva was gently grasped with Dumont #4 forceps, and the injection was made using a 33 G needle and a Hamilton Syringe. After dispensing the syringe contents, the syringe needle was slowly withdrawn. Following the injection procedure, 1 drop of Ofloxacin ophthalmic solution was applied topically to the ocular surface with eye lube.

### Parameters

Mortality and morbidity were evaluated done once daily along with cage-side observations with particular attention paid to eyes. Ocular examination was performed using a slit lamp biomicroscope to evaluate ocular surface morphology at the timepoints indicated herein. Both color and cobalt blue (EGFP expression) fundus imaging was done on both eyes of all animals at the timepoints in the experimental design table. Animals were given a cocktail of tropicamide (1.0%) and Phenylephrine (2.5%) topically to dilate and proptose the eyes, and topical eye anesthetic was applied to the eyes (proparacaine 0.5% or similar). Color fundus photography were followed by cobalt blue photography. Following final imaging procedures on Day 28, animals were euthanized via carbon dioxide asphyxiation and death was confirmed by cervical dislocation or thoracotomy.

### Ocular Tissue Collection and Processing

Following final in-life imaging procedures, the eyes were enucleated and immediately fixed in 4% paraformaldehyde (PFA) in phosphate-buffered saline (PBS) for 4 hours at room temperature (cryosection analysis) or 30 minutes at room temperature (flat mount analysis). Eyes designated for flat mount analysis were rinsed with 1x PBS and stored at 4° C. until dissection. Eyes designated for cryosectioning are washed with PBS and stored at 4° C. until embedding.

Cryosection Analysis: Eyes are embedded immediately in 3% agarose/5% sucrose and sunk overnight in 30% sucrose at 4° C. or stored in 1x PBS until they are embedded the following day. Blocks are sectioned and processed for immunohistochemistry. Slides designated for immunohistochemistry are stained with antibodies against Rhodopsin, RPE65 and GFP, alongside DAPI for nuclear localization. Secondary antibodies can be as follows: donkey anti-mouse Cy3 (rhodopsin), donkey anti-rabbit Cy5 (RPE65), and donkey anti-chicken Cy2 (GFP).

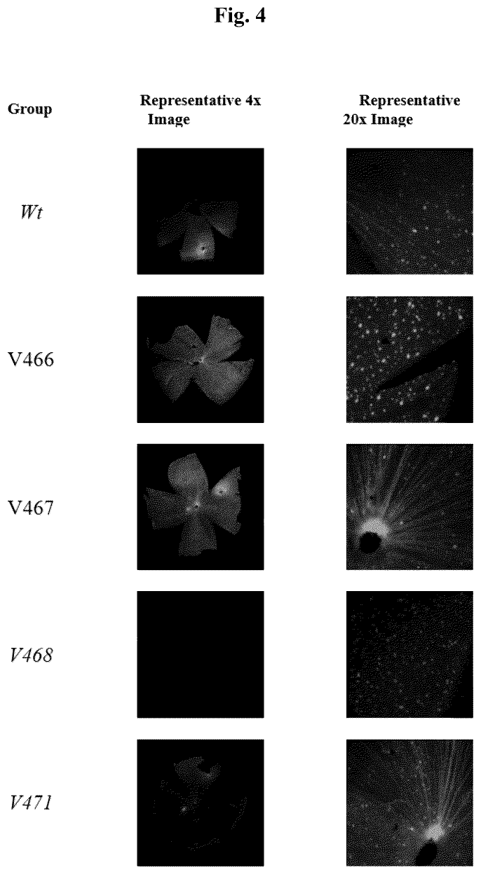

Ocular Flatmount Analysis: Using a dissecting microscope, the eye was trimmed of extraneous tissue and the anterior segment, lens and vitreous was removed. Eye cups were placed in cold methanol for 30 minutes, then rinsed with cold PBS for 15 minutes 3 times. Eye cups were placed in cold ICC containing 4′,6-diamidino-2-phenylindole (DAPI; nuclear stain) and a combination of three of the following antibodies: anti-laminin ( 1/1,000) labeling the inner limiting membrane, anti-rhodopsin clone 4D2 ( 1/500) labeling rods, anti-glutamine synthetase clone GS-6 ( 1/1,500) labeling Müller cells, PNA Lectin ( 1/40) labeling cones. Eye cups were incubated at 4° C. with gentle rotation for 4 hours and washed with cold ICC buffer. Following staining, eye cups were fixed in 4% PFA for five minutes, then rinsed once with 1x PBS. The sclera-choroid/RPE complexes were removed from the retina using fine curved scissors. The retina was flat mounted, covered and sealed. Two-dimensional (2D) fluorescent microscopy images are acquired, digitized, and analyzed using Image ProPlus or Cell Sens (Olympus) software and post-acquisition analysis is performed with ImageJ or Cell Sens software, FIG. 4.

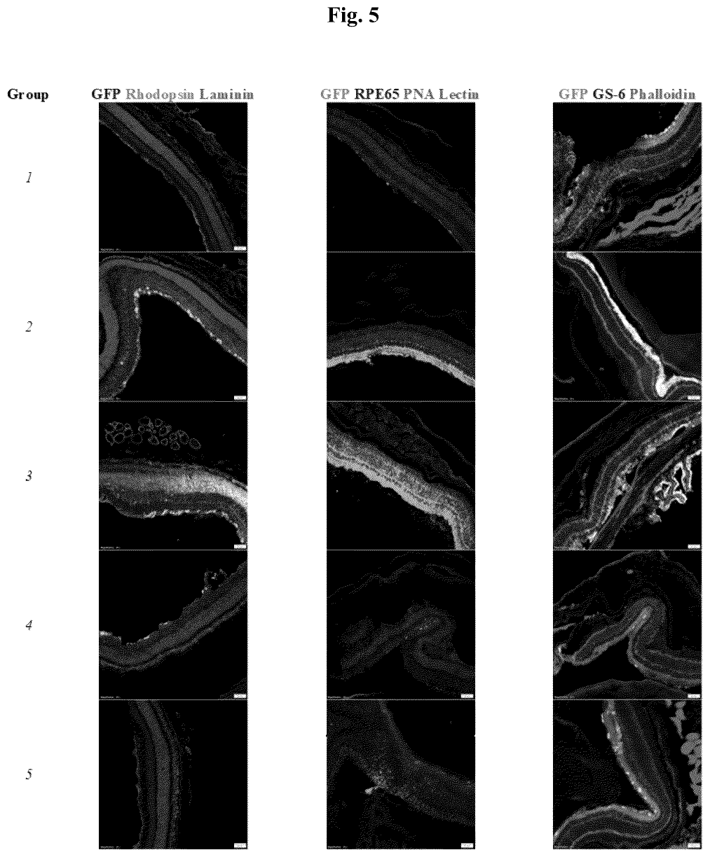

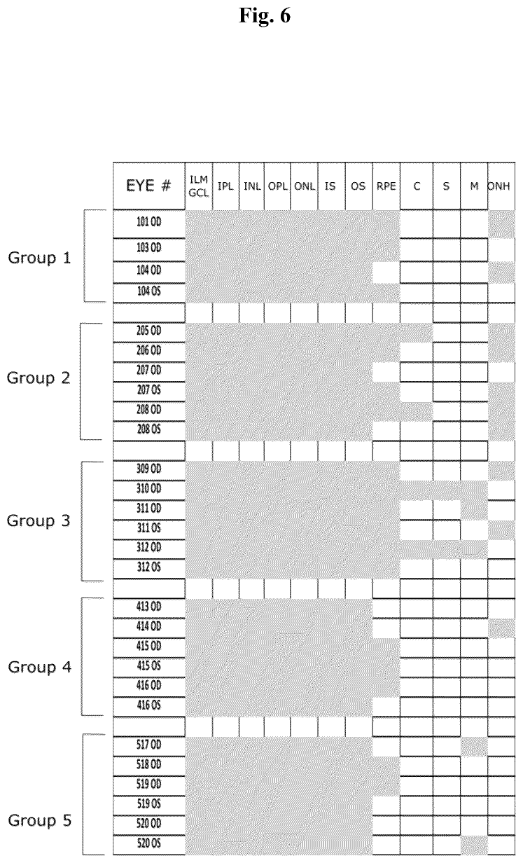

Cryosection Analysis: Frozen blocks were cryosectioned (14 μm thickness) and processed for immunohistochemistry. Each slide designated for immunohistochemistry was stained with three different cocktails containing antibodies against GFP and different markers of the retinal cell layers: cocktail 1) GFP, rhodopsin and laminin; 2) GFP, RPE65 and PNA Lectin; 3) GFP, glutamate synthetase GS-6 and phalloidin. The results are shown in FIG. 5. A graphical representation of GFP expression across all three antibody cocktails and all eyes imaged is shown in FIG. 6.

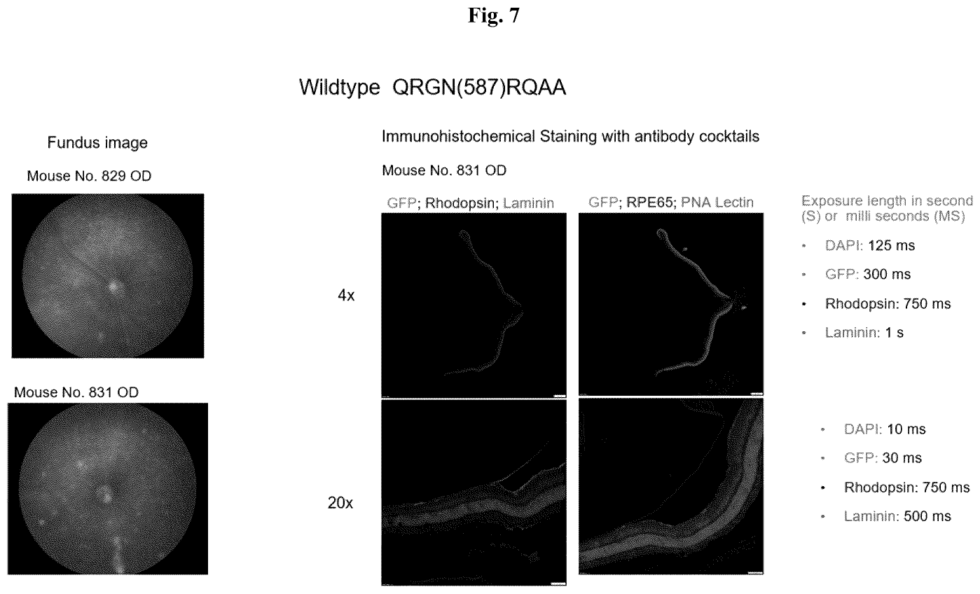

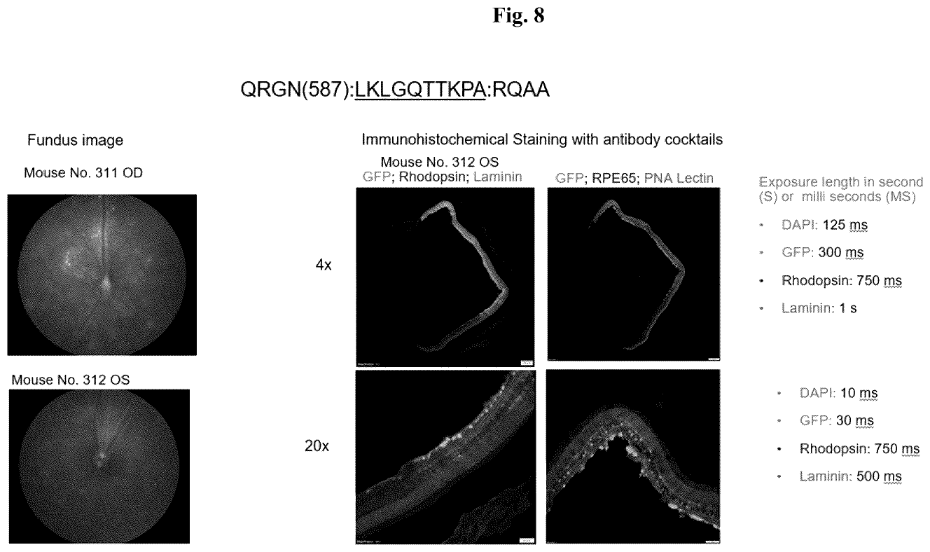

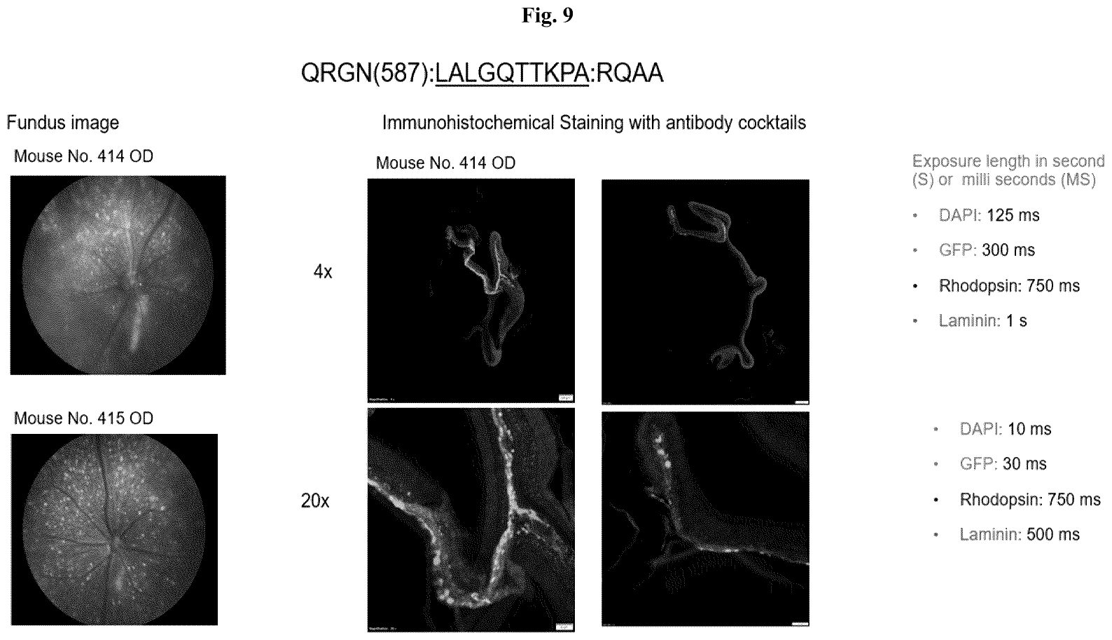

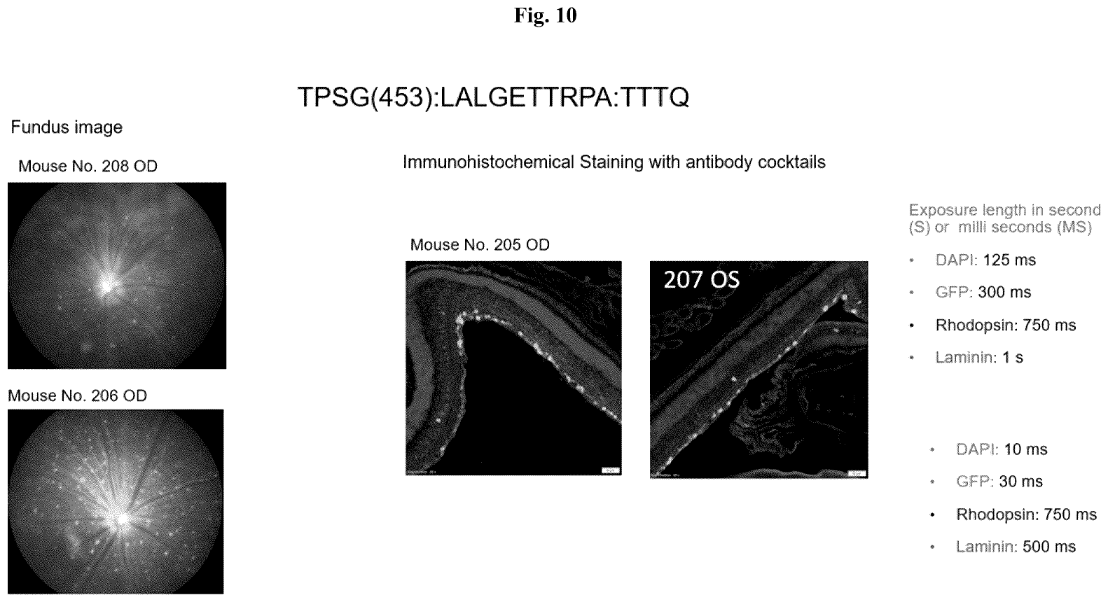

FIG. 7-FIG. 11 and Table 9-Table 11 illustrate in vivo imaging of the mouse eye administered with either wild type (wt) AAV vector or a modified AAV2 vector described herein. FIG. 7 illustrates Fundus image and IHC staining of the mouse eye administered with wild type AAV vector. The wt AAV2-GFP did not lead to enough GFP expression as shown both the fundus imaging and IHC section. FIG. 8 illustrates in vivo fundus imaging and IHC staining of the eye, where the modified AAV2 described herein (AAV2-AMI053-GFP comprising modified AAV2 with LKLGQTTKPA (SEQ ID NO: 13) inserted after amino acid residue 587) was administered via IVT route to the eye of a mouse. The modified AAV2 exhibited GFP expression in fundus imaging and IHC sections with GFP signal detected in multiple layers of cells. FIG. 9 illustrates in vivo fundus imaging and IHC staining of the eye, where the modified AAV2 described herein (AAV2-AMI054-GFP comprising modified AAV2 with LALGQTTKPA (SEQ ID NO: 14) inserted after amino acid residue 587) was administered via IVT route to the eye of a mouse. The modified AAV2 exhibited GFP expression in fundus imaging and IHC sections with GFP signal detected in multiple layers of cells. FIG. 10 illustrates in vivo fundus imaging and IHC staining of the eye, where the modified AAV2 described herein (AAV2-V466-GFP comprising modified AAV2 with LALGETTRPA (SEQ ID NO: 6) inserted after amino acid residue 453) was administered via IVT route to the eye of a mouse. The modified AAV2 exhibited GFP expression in fundus imaging and IHC sections with GFP signal detected in multiple layers of cells. Table 10 illustrates the Fundus images of mice after 25-29 days of AAV2-VP1-GFP IVT injection.

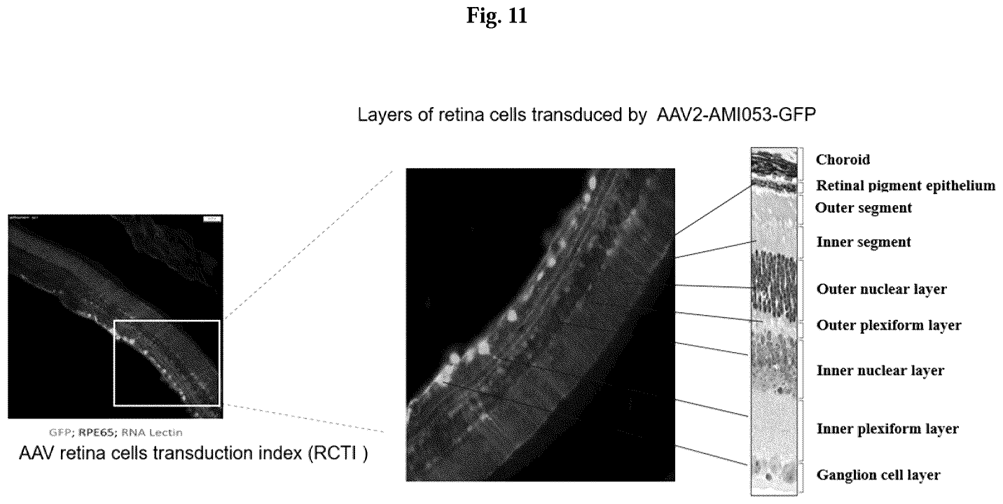

FIG. 11 illustrates AAV retina transduction index (RCTI) illustrating AAV transduction in the different optic fiber layers of the eye transduced with either the wild type (wt) or the modified AAV described herein. RCTI was calculated from the scores of color intensity of cell layers lit up by the vector administrated from intravitreous route (i.e. AAV2-AMI053-GFP transmitted across inner limiting membrane (ILM) of vitreous structure, transduced various cells via binding to the receptor on the cells. Appearance of GFP signal indicating vector entered and expressed in the cells. A 4 scales of color intensity was used to estimate the AAV transduction capacity: detected color: clear: 1 point or +; clear; bright color: 2 points or ++; strong color: 3 points; and very shinning color or +++: 4 points or ++++. Table 11 illustrates an exemplary RCTI scoring of the AAV2 transduced retina and SEQ ID NO: 129-132, in order of appearance. Table 12 illustrates an exemplary RCTI scoring of AAV2 transduction in both human cells (HEK293 cells or ARPE19 cells) or in mouse retina and SEQ ID NO: 129-132, in order of appearance.

TABLE 11
AAV2-Retina transduction IHC RCTI scoring of transduced mouse

| | | | | | | | | | | | | | | | | | | Net | |
|---|---|---|---|---|---|---|---|---|---|---|---|---|---|---|---|---|---|---|---|
| Group | AA | Insert | Eye | Staining | | | | | | | | | | | | | Total | retina | |
| # | Sequence | site | # | Cocktail | GCL | IPL | INL | OPL | ONL | IS | OS | RPE | C | S | M | ONH | score | score | Comment |
| 8 | Wild type | | 8 | 1 & 2 | 8 | 2 | 0 | 0 | 2 | 2 | 1 | 4 | 0 | 0 | 0 | 2 | 21 | 19 | Discard |
| | (Wt) | | | | | | | | | | | | | | | | | | pts |
| 9 | LALGETSR | N587 | 8 | 1 & 2 | 21 | 15 | 13 | 13 | 9 | 5 | 1 | 1 | 0 | 0 | 5 | 4 | 82 | 73 | of M |
| | PAG | | | | | | | | | | | | | | | | | | + |
| 3 | LKLGQTS | N587 | 8 | 1 & 2 | 21 | 13 | 13 | 14 | 12 | 7 | 3 | 2 | 0 | 0 | 13 | 2 | 100 | 85 | OMH |
| | KPAG | | | | | | | | | | | | | | | | | | |
| 4 | LALGQTS | N587 | 8 | 1 & 2 | 23 | 23 | 8 | 8 | 8 | 3 | 0 | 0 | 0 | 0 | 6 | 3 | 79 | 70 | |
| | KPAG | | | | | | | | | | | | | | | | | | |
| 10 | LALGETSK | N587 | 8 | 1 & 2 | 11 | 8 | 4 | 4 | 3 | 2 | 2 | 4 | 0 | 0 | 1 | 6 | 45 | 38 | |
| | PAG | | | | | | | | | | | | | | | | | | |

TABLE 12
RCTI scoring of cells or mouse retina transduced with modified AAV2

| | | In- | | | | | | | | | | | | | | | | Net | |
|---|---|---|---|---|---|---|---|---|---|---|---|---|---|---|---|---|---|---|---|
| Group | AA | sert | Eye | Staining | | | | | | | | | | | | | Total | retina | |
| # | Sequence | site | # | Cocktail | GCL | IPL | INL | OPL | ONL | IS | OS | RPE | C | S | M | ONH | score | score | Comment |
| 8 | Wild type | | 8 | 1 & 2 | 8 | 2 | 0 | 0 | 2 | 2 | 1 | 4 | 0 | 0 | 0 | 2 | 21 | 19 | Discard |
| | (Wt) | | | | | | | | | | | | | | | | | | pts |
| 9 | LALGETSR | N587 | 8 | 1 & 2 | 21 | 15 | 13 | 13 | 9 | 5 | 1 | 1 | 0 | 0 | 5 | 4 | 82 | 73 | of M |
| | PAG | | | | | | | | | | | | | | | | | | + |
| 3 | LKLGQTS | N587 | 8 | 1 & 2 | 21 | 13 | 13 | 14 | 12 | 7 | 3 | 0 | 0 | 0 | 13 | 2 | 100 | 85 | OMH |
| | KPAG | | | | | | | | | | | | | | | | | | |
| 4 | LALGQTS | N587 | 8 | 1 & 2 | 23 | 23 | 8 | 8 | 8 | 3 | 0 | 0 | 0 | 0 | 6 | 3 | 79 | 70 | |
| | KPAG | | | | | | | | | | | | | | | | | | |
| 10 | LALGETSK | N587 | 8 | 1 & 2 | 11 | 8 | 4 | 4 | 3 | 2 | 2 | 4 | 0 | 0 | 1 | 6 | 45 | 38 | |
| | PAG | | | | | | | | | | | | | | | | | | |

As showing in FIG. 7-FIG. 11 and Table 10-Table 12, N54 peptide (LALGQTTKPA) can be inserted into VP1 of all AAV vectors to enhance the vector transmitting across inner limiting membrane (ILM) and expression in retina cells. It was found that VFGF-Trap protein was expressed at highest level induced by AAV6.N54-VEGF-trap followed by AAV2.N54-VEGF-trap, and by AAV1.N54-VEGF-trap in HEK293 cells. In ARPE-19 cells, the VEGF-Trap protein was expressive similarly via both AAV2.N54-VEGF-trap and AAV6.N54-VEGF-trap.

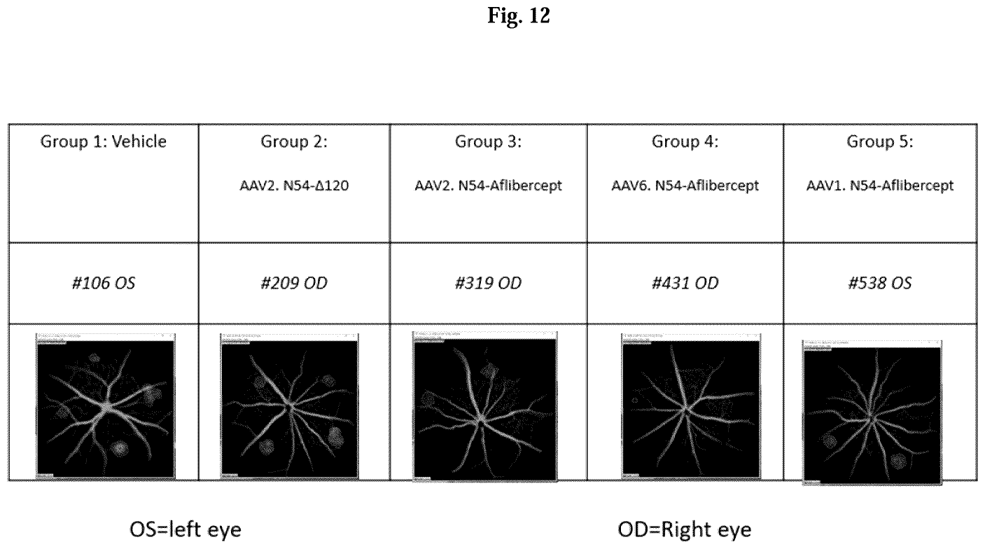

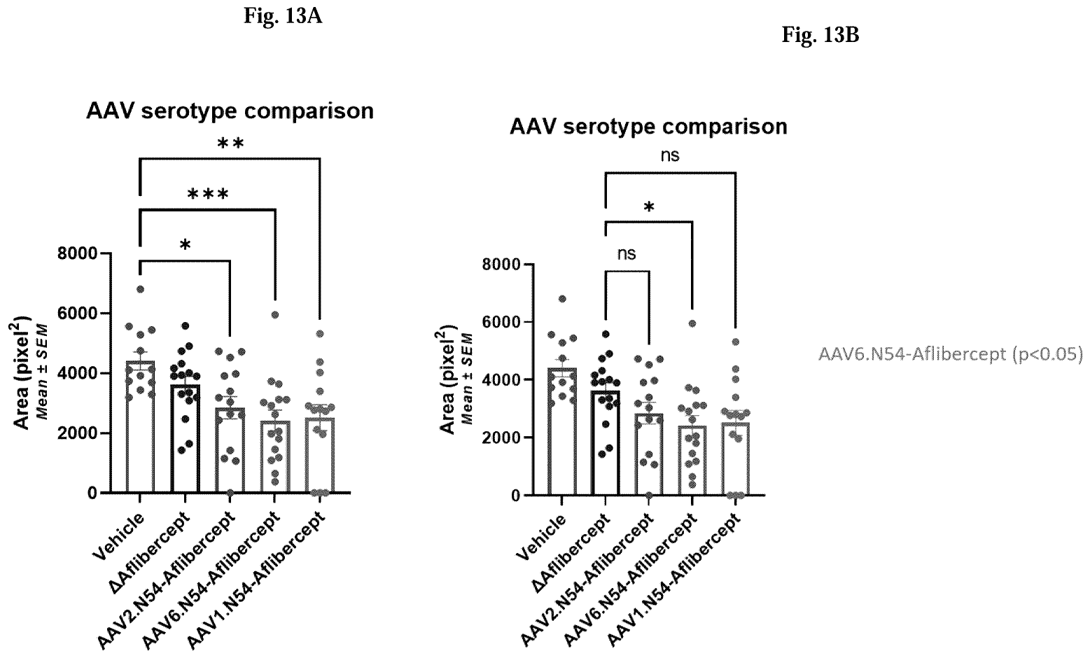

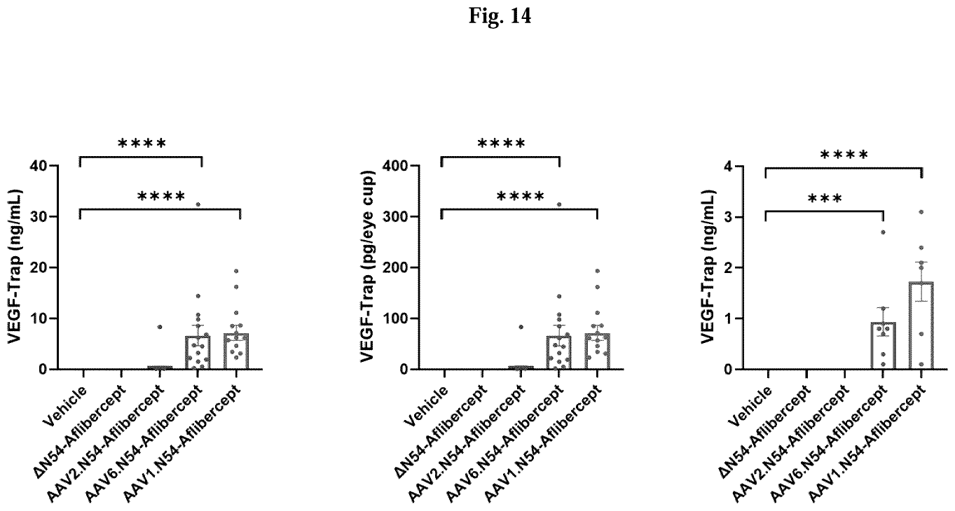

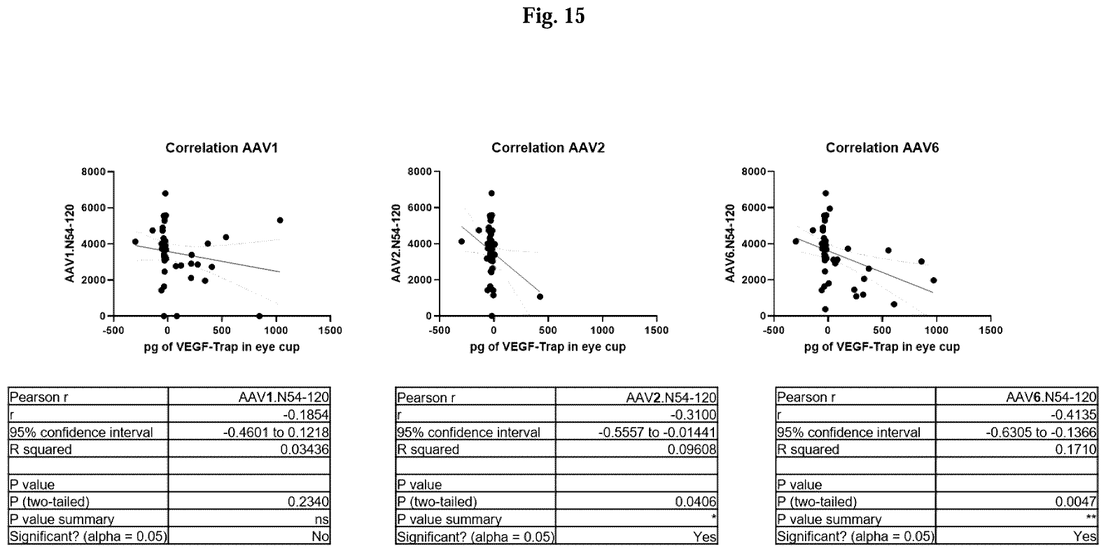

FIG. 12-FIG. 15 and Table 12-Table 16 illustrate LCNV studies conducted with mouse transduced with wild type AAV vector or with the modified AAV vector described herein. FIG. 12 illustrates the LCNV study, where modified AAV was administered to the mouse prior to the LCNV. AAV1, AAV2, or AAV6 was modified with insertion of LALGQTTKPA (SEQ ID NO: 14) in the VP1 capsid and carried Aflibercept (VEGF-Trap) as the transgene for delivery to the eye of the mouse. Modified AAV1 (Group 5, AAV1. N54-Aflibercept) comprises LALGQTTKPA (SEQ ID NO: 14) inserted after S588 amino acid residue of the VP1 capsid. AAV2 (Group 3, AAV2. N54-Aflibercept) comprises LALGQTTKPA (SEQ ID NO: 14) inserted after N587 amino acid residue of the VP1 capsid. AAV6 (Group 4, AAV6. N54-Aflibercept) comprises LALGQTTKPA (SEQ ID NO: 14) inserted after S588 amino acid residue of the VP1 capsid. FIG. 13A illustrates area of choroidal neovascularization in the eye of the LCNV study. ΔAflibercept was a sham control, where AAV comprising disruption in open reading frame was administered to the eye of the mouse. Compared to the vehicle group, all three modified AAV groups showed significant decrease in area of choroidal neovascularization in the eye (AAV6.N54-Aflibercept, p<0.001; AAV1.N54-Aflibercept, p<0.01; and AAV2.N54-Aflibercept, p<0.05). FIG. 13B illustrates that the group administered with AAV6.N54-Aflibercept continued to show significant decrease in in area of choroidal neovascularization in the eye (AAV6.N54-Aflibercept, p<0.05). FIG. 14 illustrates aflibercept concentration in ocular and serum samples obtained from the LCNV study mouse. FIG. 15 illustrates fluorescein angiography (FA) analysis of retina Aflibercept protective effect against retina damage induced by LCNV in the mouse. Correlation of Aflibercept concentration in eye cup versus retina injury caused by lasering was inversely correlated. The most pronounced decrease LCNV area in the AAV6.N54-Aflibercept group corresponded to the increased VEGF-Trap expressed mediated by the modified AAV6 as showing FIG. 3C, FIG. 3D, Table 13, and Table 14.

TABLE 13
Aflibercept expression level in mice by 3 modified AAV vectors

| | | Eye Cup (Retina) | | Serum | |
|---|---|---|---|---|---|
| Serotype | Dose/eye (IVT) | ng/mL | ng/eye cup | ng/mL | ng/animal |
| AAV2.N54-Δ120 | 1.6e+10 | <0.01 | <0.01 | <0.01 | <0.01 |
| Sham vector | vg/eye | | | | |
| AAV1.N54-Aflibercept | 4.8e+8 vg | 7.2 ± 5.3 | 0.3 ± 0.3 | 2.4 ± 0.5 | 0.6 ± 0.1 |
| AAV2.N54-Aflibercept | 4.8e+8 vg | 0.1 ± 2.2 | 0.01 ± 0.1 | Not detectable | 0.1 ± 0.03 |
| AAV6.N54-Aflibercept | 4.8e+8 vg | 6.6 ± 8.1 | 0.31 ± 0.3 | 1.3 ± 0.95 | 0.3 ± 0.2 |

TABLE 14
Advantages of modified AAV6 over modified AAV2

| AAV serotype | AAV2 | AAV6 | AAV1 |
|---|---|---|---|
| Vector | scAAV2.N54- | scAAV6.54- | scAAV1.54- |
| | Aflibercept | Aflibercept | Aflibercept |
| VP1 insertion site | N587 | S588 | S588 |
| Clinical trial | Popular in ocular | Never tested in | |
| | disease | ocular disease | |
| VEGF-trap | 881.0 ± 434.7 | 908.4 ± 842.1 | 142.1 ± 82.3 |
| expressed in ARPE- | | | |
| 19 culture (ng/ml) | | | |
| VEGF-trap in mouse | 0.01 ± 0.1 | 0.31 ± 0.3 | 0.3 ± 0.3 |
| retina (ng/eye cup) | | (30×) | |
| VEGF-trap in mouse | Not detectable | 1.3 ± 0.95 | 2.4 ± 0.5 |
| serum (ng/mL) | | | |
| LCNV | | ~20% | ~15% |
| | | outperformed | outperformed |
| On-going studies | NHP | NHP | Stop? |

Table 15 and Table 16 illustrate exemplary additional studies to determine the localization of the AAV vector and the expression of the cargo (e.g., Aflibercept) of the AAV vector after being administered to the eye. The study of Table 15 can be at least partially based on utilizing Fundus images and IHC described herein. The study of Table 16 can be at least partially based utilizing LCNV, FA, OCT, FP, histology, and IHC.

TABLE 15
AAV vector locality study

| | | | Test | | | |
|---|---|---|---|---|---|---|
| | | No. of | Article/Dose | | Experimental | |
| Group | Serotype | Animals | OU | Volume/Route/Day | Endpoints | Euthanasia |
| 1 | AAV2 | 2 | AAV2.N54- | 50 μl/IVT/Day 0 | Ocular | Day 28^{§} |
| | | | mGFP | | examinations: | Optional: n = 2 |
| | | | 1E+11 vg/eye | | Baseline and | eyes/group** will be |
| 2 | AAV6 | 2 | AAV6.N54- | | Days 7, 21 and | collected for GFP IHC |
| | | | mGFP | | 28 | All remaining eyes |
| | | | 1E+11 vg/eye | | Color and | (including naïve spare |
| 3 | AAV2.7m8 | 2 | AAV2.V226- | | cobalt blue | if not used): Snap |
| | | | mGFP | | fundus: Day 21 | freeze and |
| | | | (+Control) | | (Acquisition | collect/homogenize |
| | | | lell vg/eye | | settings will be | vitreous humor |
| | | | | | set to Group 4) | |

TABLE 16
AAV vector efficacy and expression study

| | | No. of | | | Treatment | CNV | Experimental |
|---|---|---|---|---|---|---|---|
| Group | I.D. | Animals | Treatment/ Dose | Volume/Route | Day | Induction | Endpoints |
| 1 | Null | 6 | AAV2.N54-Δ | 50.0 μL | Day −28 | OU: CNV | DAY 14-21: |
| | Control | | Aflibercept | IVT | | Laser Day | Color/Cobalt Blue |
| | | | 2e+12 vg/eye | | | 0 | Fundus Imaging: |
| 2 | Test | 6 | AAV2.N54- | | | | Day 21, prior to FA |
| | Article | | Aflibercept | | | | Fluorescein |
| | | | 2e12 vg/eye | | | | angiography (FA), |
| 3 | Null | 6 | AAV6.N54- | | | | FP, OCT: n = 6 |
| | | | ΔAflibercept | | | | Terminal blood |
| | | | 2e+12 vg/eye | | | | collection (serum): |
| 4 | Test | 6 | AA6.N54 | | | | n = 6/group |
| | Article | | Aflibercept | | | | Flatmount |
| | | | 2e12 vg/eye | | | | analysis: n = 4 |
| | | | | | | | animals (6 |
| | | | | | | | eyes)/group |
| | | | | | | | Dissect and |
| | | | | | | | homogenized whole |
| | | | | | | | eye for ELISA: n = 2 |
| | | | | | | | animals (4 |
| | | | | | | | eyes)/group |
| 5 | | 4 | Aflibercept | 50.0 μL | Day -3 | N/A | DAY 14-21 |
| | | | | IVT | | | Terminal blood |
| | | | | | | | collection (serum): |
| | | | | | | | n = 4 animals |
| | | | | | | | Dissect and |
| | | | | | | | homogenize whole |
| | | | | | | | eye for ELISA: n = 2 |
| | | | | | | | animals (4 eyes) |

### Example 8. In Vivo Testing of AAV2.N54 Capsid for Intravitreal Delivery in Weanling Farm Pig Model

Intravitreal delivery of gene therapy drugs for treatment of ocular diseases is a preferred method of choice, because it does not cause retinal detachment, can lead to pan-retinal transduction, and can be performed under local anesthesia in out-patient clinics. Wild type AAV vectors trans-crosses the inner limiting membrane (ILM) of the eyes poorly for transducing the tissues. As such, modified AAV capsids capable of penetrating the ILM are needed for successful delivery of the gene therapy drugs to the intended targets. Example 8 presents the results of the engineered AAV2.N54 vector capable of delivering green fluorescent protein (GFP) deep into the eye tissues in the weanling farm pig model.

### Cloning and AAV Vector Production

AAV2 wild type capsid gene was used as starting material to engineer novel AAV variant capsids. Briefly, several peptide sequences were engineered, reverse-transcribed into optimized DNA sequences and cloned into specific locations of the AAV2 wild type capsid gene to create AMI053, AMI054, AMI101, AMI104, and AMI105 plasmids. Recombinant baculoviruses (rBV) were generated and used together with rBV-CMV-GFP to co-infect SP9-V432-AG cells to produce AAV vectors expressing GFP. The AAV vectors were purified with two rounds of cesium chloride (CsCl) density gradient ultracentrifugation. The CsCl was removed through 2 rounds of desalting with PD-10 desalting columns and formulated into formulation buffer.

### Experimental Design

The animals were divided into 6 groups with 2 animals per group for intravitreal injection of different AAV vectors according to the Table 17.

TABLE 17
Experimental design for intravitreal injection of different AAV vectors into the pig eyes

| | | Test | | | |
|---|---|---|---|---|---|
| | No. of | Article/Dose | | | |
| Group | Animals | OU | Volume/Route/Day | Experimental Endpoints | Euthanasia |
| 1 | 2 | AMI053 | 25 μl/IVT/Day 0 | Ocular examinations: | Day 28^{§} |
| | | 1e+11 vg/eye | | Baseline, and Days 7, 21 | Optional: n = 2 |
| 2 | 2 | AMI054 | | and 28 | eyes/group ** will be |
| | | 1e+11 vg/eye | | Color and cobalt blue | collected for GFP |
| 3 | 2 | AMI101 | | fundus: Day 21 | IHC |
| | | 1e+11 vg/eye | | (Acquisition | |
| 4 | 2 | AMI104 | | settings will | |
| | | 1e+11 vg/eye | | be set to Group 6) | |
| 5 | 2 | AMI105 | | | |
| | | 1e+11 vg/eye | | | |
| 6 | 2 | V226 | | | |
| | | (+ Control) | | | |
| | | 1e+11 vg/eye | | | |

### Surgical Procedures

Test articles were produced in a ready-to-inject format and stored at <−70° C. until use. Thirty minutes prior to injection, each test article was thawed and briefly centrifuged (1,000 rpm×10 seconds) to collect the liquid in the bottom of the tube, then heated in a 37° C. water bath for 20 minutes and vortexed to reduce virus aggregation; each tube was shaken to condense the liquid at the bottom of the tube.

On the day prior to procedures, animals were fasted overnight prior to anesthetic administration. On day of intravitreal injections, buprenorphine (0.01-0.05 mg/kg) was given intramuscularly (IM) to each animal prior to the injection. Approximately 15 minutes prior to anesthetic injection, animals were given atropine (0.01-0.05 mg/kg, IM). Animals were then anesthetized with ketamine/dexmedetomidine (IM) for the injections and the eyes were aseptically prepared using topical 5% betadine solution, followed by rinsing with sterile saline, and application of one drop of 0.5% proparacaine HCL and 10% phenylephrine HCL. The conjunctiva was gently grasped with colibri forceps, and the injection (27-30G needle) made 2-3 mm posterior to the superior limbus (through the pars plana) with the needle directed slightly posteriorly to avoid contact with the lens. The injection was made, and the needle was slowly withdrawn. Following the injection procedure, 1 drop of antibiotic ophthalmic solution was applied topically to the ocular surface. Animals then received atipamezole IM to reverse the effects of dexmedetomidine and were allowed to recover normally from the procedure.

### Cage Side Observations

Morbidity and mortality were observed daily along with cage-side observations, with particular attention paid to both eyes. There was no morbidity or mortality during the course of this study.

### Ocular Examinations

Mydriasis for ocular examinations was done using topical 1% tropicamide HCL (one drop in each eye 15 minutes prior to examination). Complete ocular examination (modified Hackett and McDonald) using a slit lamp biomicroscope and indirect ophthalmoscope to evaluate ocular surface morphology, anterior segment and posterior segment inflammation, cataract formation, and retinal changes was conducted according to the experimental design table above. No ocular findings were noted during these evaluations. All groups had scores of zero (0) at all timepoints evaluated and no findings were noted.

### Fundus Imaging

The day before fundus imaging procedures on Day 21, animals were fasted overnight prior to anesthetic administration. On days as indicated in the study design table, animals were anesthetized with ketamine/dexmedetomidine (IM) and were imaged using the Ret Cam 3 (Natus). Color images were acquired first followed by cobalt blue images. Settings were held constant across all groups. Acquisition settings were set to Group 6 (V226) positive control animals.

### Results

No abnormal ocular findings were observed during the whole experiment. In order to make sure that all animals were in good conditions according to the study protocol, mydriasis for ocular examinations was done using topical 1% tropicamide HCL (one drop in each eye 15 minutes prior to examination). Complete ocular examination (modified Hackett and McDonald) using a slit lamp biomicroscope and indirect ophthalmoscope to evaluate ocular surface morphology, anterior segment and posterior segment inflammation, cataract formation, and retinal changes was conducted according to the experimental design table above. No ocular findings were noted during these evaluations.

### Demonstration of GFP Expression in the Pig Eye Tissues

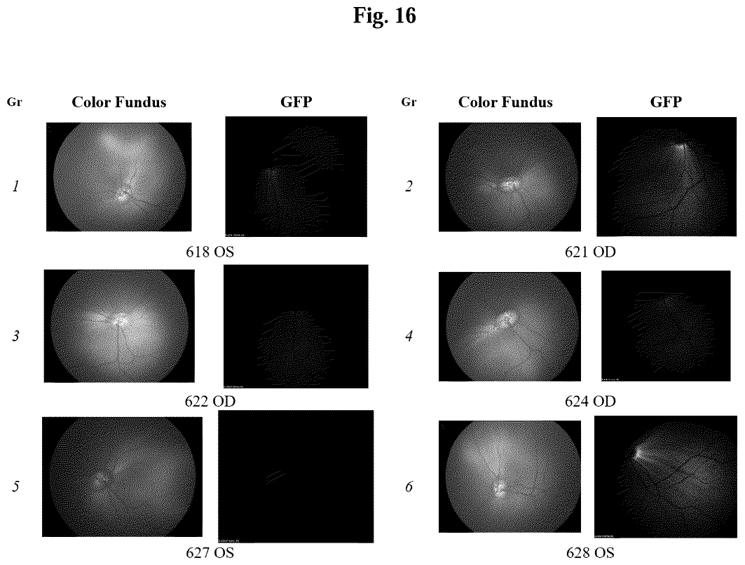

Twenty-one days after intravitreal injection of different AAV vectors carrying GFP gene into the pig eyes, GFP expression was observed in the eyes of all 6 groups of animals based on the fundus imaging. However, the GFP expression levels in groups 1, 3, 4, and 5 were low, whereas groups 2 and 6 had much higher GFP expression levels as indicated in the fundus imaging (FIG. 16). Further examination of the fundus images with a scoring scale show that group 6 had the highest GFP expression represented by +++ scores in both animals, followed by group 2 that had five ++ and one single + scores. Group 1, 3, and 4 had similar expression level of one + score. Group 5 had the lowest expression level (Table 18).

TABLE 18
Scoring scales showing the GFP expression levels in different
animal groups

| Group # | GFP Expression Score (Each Cell Represents | | | |
|---|---|---|---|---|
| | an Individual Eye) | | | |
| | Animal #1 | | Animal #2 | |
| | OS | OD | OS | OD |
| 1 | + | + | + | +/− |
| 2 | + | ++ | ++ | ++ |
| 3 | + | + | + | + |
| 4 | + | + | + | + |
| 5 | − | +/− | − | − |
| 6 | +++ | +++ | +++ | +++ |

Demonstration of Deep Penetration of AAV2.N54 Vector into Eye Tissues by IHC

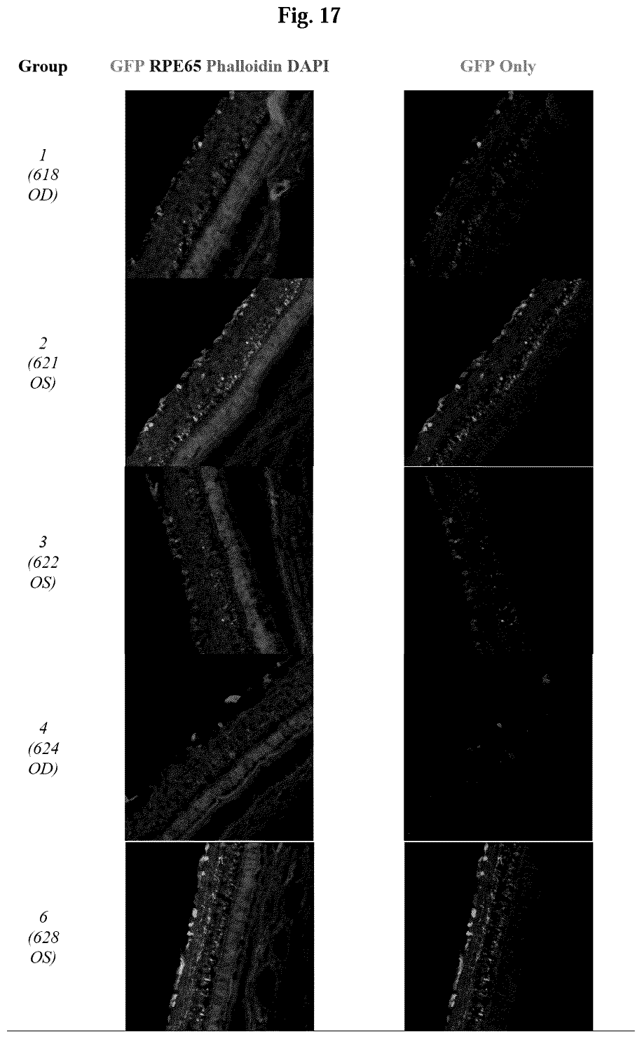

Cryosections (14 μm) were evaluated for the localization of GFP, RPE65, phalloidin, and DAPI in the eye tissues and the results are shown in FIG. 17. Merged images are shown on the left and pullouts of the GFP channel are shown on the right. These results indicate that group 2 animals (621 OS) injected with engineered AAV produced from AMI054 (designated as AAV2.N54) showed stronger and deeper penetration of GFP in the eye tissues when compared with the control group 6 (628 OS) injected with AAV made from V226. These results were further confirmed when the cryosections were examined with scoring scales in more details (Table 19). GFP expression was scored across all retinal and extra-retinal layers, for a survey of ocular GFP expression across all eyes enrolled in cryosection analysis; each eye is represented by a single row. Dark grey cells (+++) indicate where GFP expression was the highest; Medium green cells (++) indicate where there was moderate GFP expression; Medium grey cells (+) indicate where there was weak GFP expression; Light grey cells (−) indicate where GFP was absent. Abbreviations: GCL/ILM—ganglion cell layer/Inner limiting membrane; IPL—inner plexiform layer; INL—inner nuclear layer; OPL—outer plexiform layer; ONL—outer nuclear layer; IS—inner segment; OS—outer segment; RPE—retinal pigmented epithelium; C—choroid; S—sclera.

TABLE 19
Scoring scale showing GFP expression across both antibody cocktails and all
cryosectioned eyes

| Group # | Eye # | GCL | IRL | INL | OPL | ONL | IS | OS | RPE | C | S |
|---|---|---|---|---|---|---|---|---|---|---|---|
| 1 | 618 OD | +++ | +++ | +++ | +++ | ++ | ++ | ++ | − | − | − |
| 1 | 618 OS | +++ | ++ | ++ | ++ | ++ | ++ | ++ | + | − | − |
| 2 | 620 OD | ++ | ++ | +++ | +++ | ++ | ++ | ++ | + | − | − |
| 2 | 620 OS | ++ | ++ | +++ | +++ | ++ | ++ | ++ | − | − | − |
| 2 | 621 OD | +++ | +++ | +++ | +++ | ++ | ++ | ++ | + | − | − |
| 2 | 621 OS | +++ | +++ | +++ | +++ | +++ | +++ | +++ | + | − | − |
| 3 | 622 OS | +++ | ++ | ++ | ++ | ++ | ++ | ++ | + | − | − |
| 3 | 623 OS | +++ | ++ | ++ | ++ | ++ | ++ | ++ | + | − | − |
| 4 | 624 OD | ++ | + | ++ | ++ | + | + | + | − | − | − |
| 4 | 625 OD | ++ | ++ | ++ | ++ | + | + | + | + | − | − |
| 6 | 628 OS | +++ | +++ | +++ | +++ | ++ | ++ | ++ | + | − | − |

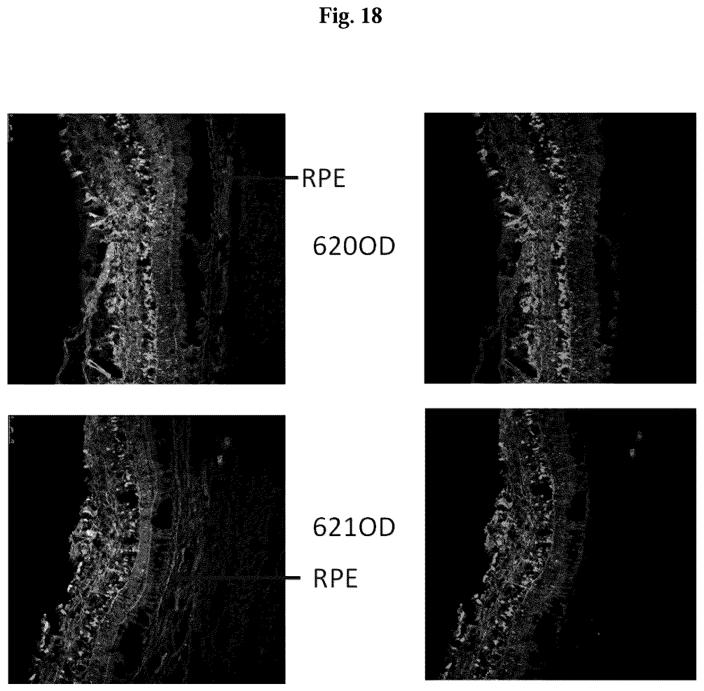

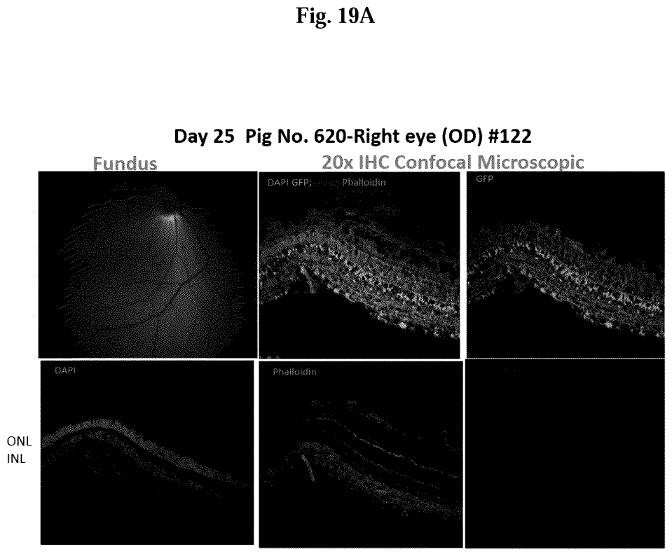

Since the cryosections examined were first performed with standard microscope, the cryosections of group 2 animals (620 OD and 621 OD) were further examined with confocal microscope. The results are shown in FIG. 18. From these sharp images we can confirm that the GFP expression cells are widely distributed from GCL to RPE layers. This clearly demonstrates that AAV2.N54 capsid can efficiently trans-cross the ILM and deeply penetrate the eye tissue up to the RPE layer of the pig eyes.

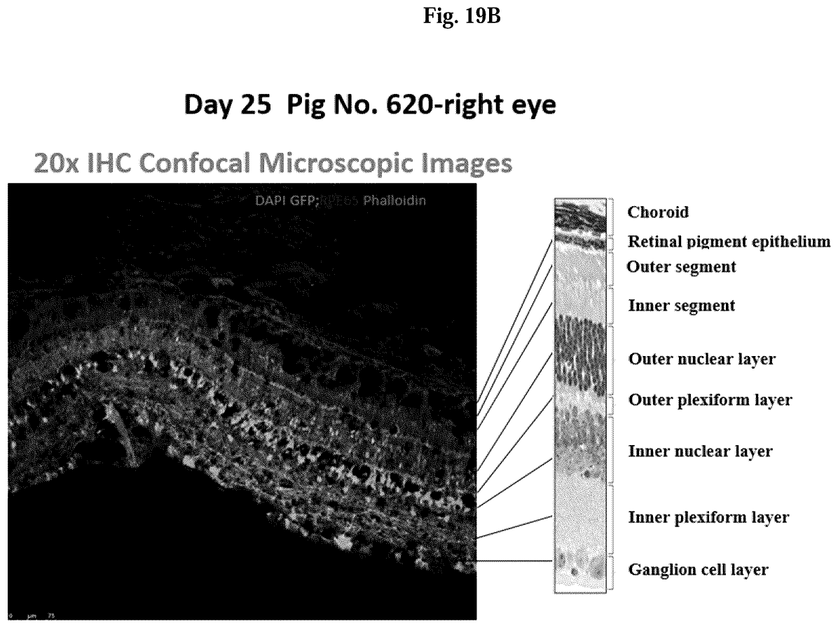

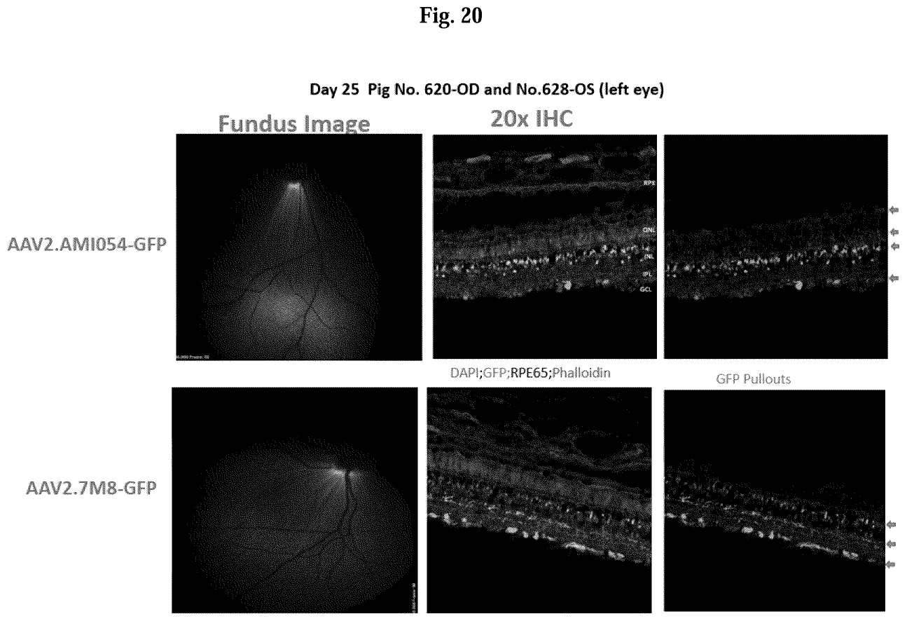

FIG. 19A illustrates intravitreous delivery of AAV2.AMI054-GFP (modified AAV2 comprising the LALGQTTKPA (SEQ ID NO: 14) insertion) in to eye of a pig. IHC staining showed GFP fluorescence in retina layers. FIG. 19B illustrates GFP expression in each retinal layer of the eye administered with AAV2.AMI054-GFP. The confocal image #122 of cryosection animal No. 620-OD was aligned with a published image of human retina Hematoxylin and Eosin (H&E) staining method. Results of the pig model study demonstrated that the AAV2.N54-GFP with N54 peptide (sequence LALGQTTKPA (SEQ ID NO: 14)) insertion into AAV2.VP1 the C-terminus of N587 position produced very delivery effect in GFP expression illustrated by strong fluorescence, deep and broad distribution throughout the retina layers and cells (FIG. 19B). The strong and wide expression of GFP indicated that the AAV2.N54 capsid delivered transgenes to corresponding layers and cells of large animal and human being. FIG. 20 illustrates a comparison of GFP expression in pig retina between eye administered with AAV2.AMI054-GFP or with AAV2.7M8-GFP (control). The retina transduced with AAV2.AMI054-GFP showed GFP expression in all retinal layers as opposed to the retina transduced with AAV2.7M8-GFP. The GFP fluorescent in the AAV2.AMI054-GFP transduced retina was twice as intense as the AAV2.7M8-GFP transduced retina. Table 20 illustrates GFP expression intensity level in human cell (HEK293 cell or ARPE19 cell), mouse model, and pig model transduced by five modified AAVs described herein: AMI053, AMI054, AMI101, AMI104, or AMI105. The increasing number of + denotes increasing GFL intensity detected in the transduced cell.

TABLE 20
GFP expression intensity in transduced human
cells, mouse model, and pig model

| Construct | Modified | HEK293 | ARPE19 | | Pig |
|---|---|---|---|---|---|
| ID | Capsid Region | Cell | Cell | Mouse | model |
| V466 | TPSG(453):LALGET | ++ | ++ | ++++ | + |
| | TRPA:TTTQ | | | | |
| AMI053 | QRGN(587):LKLGQT | ++++ | ++++ | ++++ | ++++ |
| | TKPA:RQAA | | | | |
| AMI054 | QRGN(587):LALGQT | ++++ | ++++ | ++++ | ++++ |
| | TKPA:RQAA | | | | |
| AMI101 | QRGN(587):VALGQT | ND | ND | ++++ | + |
| | TKPA:RQAA | | | | |
| AMI104 | LQRG(586):LALGQS | ND | ND | ++++ | + |
| | TKPA:NRQA | | | | |
| AMI105 | NTPS(452):LALGQT | ND | ND | ++++ | + |
| | TKPA:GTTT | | | | |

This study was conducted in weanling farm pigs (Hampshire cross) to evaluate the effect of six different AAV2 viral capsid mutants on GFP expression patterns following a single intravitreal injection in weanling pigs. No ocular findings were noted on clinical examinations at Day 7 and 21. GFP was readily observed in all groups in vivo via cobalt blue fundus imaging. GFP expression was further evaluated in Groups 1, 2, 3, 4 and 6 via cross-sectional analysis on cryosections. Group 6 (V226; positive control) presented with intense and broad GFP expression on both fundus imaging and in cross-sectional analysis. The GFP signal was the strongest from the GCL to OPL layers but less intense in the ONL to RPE layers. Group 2 (AMI054) also had the strong and broad fundus expression of GFP and expression from GCL to OS layers, a total of 7-layer strong expression. Modest differences between Groups 1 (AMI053), 3 (AMI101) and 4 (AMI104) on fundus imaging were also apparent on cross-sectional analysis with 618 OD from Group 1 having broad and bright expression through the outer plexiform layer (OPL). GFP expression in Group 3 (AMI101) was bright in the ganglion cell layer and moderate through the remaining layers. In Group 4, GFP expression was moderate to low throughout the retina. Very high expression was noted in the inner nuclear and outer plexiform layers for Groups 1, 2 and 6. Morphological analysis of these GFP positive cells indicated amacrine cell identity; future studies should utilize co-labeling approaches to evaluate whether amacrine markers co-localize with GFP. Choroidal and sclera GFP expression was not observed in any of the eyes and RPE GFP expression was faint compared to the other retinal layers. Taken together, Groups 2 (AAV2.N54) showed the broadest layers of GFP expression and out-performed group 6 (V226) by deep penetrating 7 layers of eye tissue with strong GFP expression.

### Example 9. Evaluation of Biodistribution and Transgene Expression of AAV GFP Candidates Following Intravitreal Administration in Nonhuman Primates

To evaluate the biodistribution and concentration of green fluorescence protein (GFP) expression of AAV candidates following intravitreal (IVT) administration in African green monkeys. Monkeys will undergo baseline screening to assess AAV neutralizing antibody (nAb) seronegativity, complete blood count (CBC), general well-being, and ocular health by slit lamp biomicroscopy, fundoscopy, color fundus imaging, confocal scanning laser ophthalmoscopy (cSLO), and optical coherence tomography (OCT). nAb negative monkeys with normal findings will be enrolled in the study and assigned to treatment groups (Table 21). For baseline screening and all subsequent procedures, anesthesia will be achieved with intramuscular ketamine (8 mg/kg) and xylazine (1.6 mg/kg) to effect, and pupil dilation with topical 10% phenylephrine, 1% tropicamide and/or 1% cyclopentolate.

TABLE 21
Treatment assignment

| | | | | Route | Concentration | Volume | Dose | Test Article |
|---|---|---|---|---|---|---|---|---|
| Group | N | Eye | Treatment | * | (vg/mL) | (mL) | (vg/eye) | Required |
| 1 | 1 | OU | Vehicle | IVT | — | 50 | — | (50 μL + 20 μL |
| | | | | | | | | overage) × 2 = 140 μL |
| 2 | 2 | OU | wtAAV2-mGFP | IVT | 2 × 10¹² | 50 | 1 × 10¹¹ | (50 μL + 20 μL |
| | | | | | | | | overage) × 4 = 280 μL |
| 3 | 2 | OU | AAV2.N54-mGFP | IVT | 2 × 10¹² | 50 | 1 × 10¹¹ | (50 μL + 20 μL |
| | | | | | | | | overage) × 4 = 280 μL |
| 4 | 2 | OU | wtAAV6-mGFP | IVT | 2 × 10¹² | 50 | 1 × 10¹¹ | (50 μL + 20 μL |
| | | | | | | | | overage) × 4 = 280 μL |
| 5 | 2 | OU | AAV6.N54-mGFP | IVT | 2 × 10¹² | 50 | 1 × 10¹¹ | (50 μL + 20 μL |
| | | | | | | | | overage) × 4 = 280 μL |
| 6 | 2 | OU | AAV2.V226-mGFP | IVT | 2 × 10¹² | 50 | 1 × 10¹¹ | (50 μL + 20 μL |
| | | | | | | | | overage) × 4 = 280 μL |

### Intravitreal (IVT) Dosing

Topical proparacaine 0.5% will be administered, allowing 30 seconds to take effect, and an eye speculum placed, then the ocular surface rinsed with 5% Betadine solution followed by sterile 0.9% saline. IVT injections will be performed in both eyes (OU) according to the treatment assignment (Table 21) using a 31-gauge 5/16-inch needle/syringe (Ulticare VetRx U-100, or equivalent) inserted inferotemporally at the level of the ora serrata ˜2 mm posterior to the limbus. Following injection, a topical antibiotic ophthalmic ointment (neomycin, polymyxin, bacitracin or equivalent) will be administered.

### Systemic Immunosuppression Regimen

Animals will receive intramuscular (IM) delivery of methylprednisolone (8 mg/kg) on Day −1, then weekly for three weeks (Table 22).

TABLE 22
Study schedule

| | | | Study Interval | | | | | | |
|---|---|---|---|---|---|---|---|---|---|
| | | | Week | Week | Day | | | | |
| Event | # | Eye | −3 | −1 | −1 | Day 0 | Day 7 | Day 21 | Day 28+ |
| nAb serum | 14 | — | X | — | — | — | — | — | — |
| CBC | 11 | — | X | — | — | — | — | — | — |
| Physical exam | 11 | — | X | — | — | — | — | — | — |
| Body weight | 11 | — | X | X | — | X | X | X | X |
| Intravitreal dosing | 11 | OU | — | — | — | X | — | — | — |
| Methylprednisolone* | 11 | — | — | — | X | — | X | X | X |
| Tonometry | 11 | OU | — | X | — | — | X | X | X |
| Slit lamp exam | 11 | OU | X | — | — | — | X | X | X |
| Color fundus | 11 | OU | — | X | — | — | — | X | — |
| imaging | | | | | | | | | |
| Fluorescence | 11 | OU | — | X | — | — | — | X | — |
| imaging | | | | | | | | | |
| cSLO IR/AF | 11 | OU | — | X | — | — | — | X | — |
| imaging | | | | | | | | | |
| OCT | 11 | OU | — | X | — | — | — | X | — |
| Vitreous collection | 11 | OU | — | — | — | — | — | — | X |
| Eye collection | 11 | OU | — | — | — | — | — | — | X |

X indicates event occurring in all groups
*Methylprednisolone (8 mg/kg IM) administered weekly
+Animals will be euthanized following review of Day 28 exam data, which may fall on a subsequent day.

### Tonometry

At designated time points (Table 22) intraocular pressure (TOP) will be measured OU using a TonoVet tonometer set to the dog (d) calibration setting. Three measures will be taken from each eye and the mean TOP calculated.

### Slit Lamp Exam

At designated time points (Table 22) both eyes (OU) will be examined by slit lamp biomicroscopy. Scoring will be applied to qualitative clinical ophthalmic findings using a nonhuman primate ophthalmic scoring system and summary score derived from exam components.

### Fundus Imaging

At designated time points (Table 22) color anterior and fundus imaging and fluorescent fundus imaging to detect GFP expression will be performed OU with a 50° field of view centered on the macula with additional peripheral images acquired in each quadrant using a Topcon TRC-50EX retinal camera with Canon 6D digital imaging hardware and New Vision Fundus Image Analysis System software. Color fundus photos will be captured with shutter speed (Tv) 1/25 sec, ISO 400 and flash 18. Monochromatic and color fluorescent images captured with exciter and barrier filters engaged (480 nm exciter/525 nm barrier filter), Tv ⅕ sec, ISO 3200 and flash 300. Fluorescence photographs will be evaluated using a scoring system to define extent of GFP expression with quantitative analysis applied as appropriate where a score of 0=absent; 1=trace; 2=slight; 3=moderate; 4=bright and 5=intense in the foveal, peripheral, and perivascular regions of the eye.

Optical Coherence Tomography (OCT) and Confocal Scanning Laser Ophthalmoscopy (cSLO)

At designated time points (Table 22) OCT and cSLO will be performed OU using a Heidelberg Spectralis OCT HRA (or OCT Plus), employing the Heyex TruTrack and AutoRescan follow-up imaging function referencing the baseline images. cSLO infrared (IR) and autofluorescence (AF) retinal images will be obtained using the wide-angle 50° lens followed by an overall OCT volume scan of the entire macula at a dense scan interval. Images will be qualitatively evaluated with quantitative analysis of the OCT retinal thickness data and GFP expression, as appropriate. When permitted by pupil size and media clarity, peripheral fields of view will be obtained with the wide-angle lens and/or the Composite Image function will be employed to maximize the area of the retina that is captured in each image file.

### Clinical Observations

General wellbeing will be confirmed twice daily by cage side observations beginning one week prior to dosing. Food consumption and overall appetite will be assessed by visual inspection of the feed pan and cage floor prior to cage washing before daily feeding. Animals will be evaluated for signs of ocular inflammation, including swelling, discoloration, squinting, or eye rubbing.

### Body Weights

Animal weights will be measured at designated time points (Table 22).

CBC with Differential

At designated time points (Table 22), 0.5-1 mL whole blood will be collected and transferred directly to K3EDTA lavender-top tubes for determination of CBC with differentials by Hemavet analyzer.

### Serum Collection

Whole blood (3 mL) will be collected via the femoral or saphenous vein at designated time points (Table 22). Blood will be transferred to vacutainer tubes (in the absence of anticoagulant) and incubated at room temperature for approximately 1 hour before centrifugation 4000 rpm for 10 minutes at 4° C. and isolation of serum aliquots (˜0.5 mL×2 aliquots per time point). Aliquots will be stored and shipped below −70° C. to a designated laboratory for nAb analysis.

### Termination

After confirming final image quality prior to the defined terminus, monkeys will be sedated intramuscularly with ketamine (8 mg/kg) and xylazine (1.6 mg/kg) to effect and euthanized with sodium pentobarbital (100 mg/kg IV).

### Eye Collection

After placement of a suture at the limbus at the 12 O'clock position globes will be enucleated and excess orbital tissue will be trimmed. Eye collection for hematoxylin and eosin (H&E) and immunohistochemistry (IHC) staining: After withdrawing ˜200 uL of vitreous, three eyes from group 2-6 and one eye from group 1 will be fixed by injection of ˜400 uL of 4% paraformaldehyde (PFA) into the vitreous chamber followed by immersed in 4% PFA for 24 hours at room temperature, then transferred to PBS with 0.05% sodium azide and storage and shipment at 4° C. in a Credo Cube temperature-controlled cold container to a Sponsor designated laboratory for H&E and GFP IHC staining and analysis of a minimum of 20 horizontal sections spanning macular region of the posterior pole. Vitreous will be stored and shipped below −70° C. to the Sponsor for protein analysis by ELISA. Eye collection for protein and RNA analysis: One eye from each group will be flash frozen in liquid nitrogen vapor prior to dissecting at room temperature along natural tissue planes. The anterior segment will be removed, and longitudinal cuts will then be placed in the frozen eye cup to flat mount the frozen posterior pole and collect the vitreous. Vitreous humor (˜2.5 mL full volume) will be divided into two aliquots. The flat mount will be divided into a superior and inferior hemi-flat mounts and the retina will be separated from the RPE/choroid, and each collected individually. All samples will be placed in labelled, cryovials, and stored and shipped below −70° C. to the Sponsor for protein analysis by GFP ELISA analysis and mRNA analysis by RT-PCR. Instruments will be cleaned in water then 70% isopropyl alcohol between eyes and tissues to minimize cross tissue contamination.

### Data Analysis

Statistical Methods: Data generated from protocol-defined endpoints listed in Table 22 will be collated, summarized, and analyzed as detailed in Table 23. Given sample size, descriptive statistics will be applied where appropriate.

TABLE 23
Data Deliverables

| Endpoint | Data Type | Statistics | Time points |
|---|---|---|---|
| nAb serologies | Numerical measures | Descriptive | All defined in |
| | | statistics | Table 2 |
| Hematology | Numerical measures | Descriptive | All defined in |
| | | statistics | Table 2 |
| Slit lamp | Graded clinical | Descriptive | All defined in |
| | scoring | statistics | Table 2 |
| IOP | Numerical measures | Descriptive | All defined in |
| | | statistics | Table 2 |
| Color fundus | Image appendix | N/A | All defined in |
| photographs | | | Table 2 |
| OCT and cSLO | Image appendix | N/A | All defined in |
| images | | | Table 2 |
| OCT retinal | Numerical measures | Descriptive | All defined in |
| thickness | | statistics | Table 2 |
| Protein ELISA | Numerical measures | Descriptive | Terminus |
| analyses | (Sponsor) | statistics | |
| RNA RT-PCR | Numerical measures | Descriptive | Terminus |
| analysis | (Sponsor) | statistics | |
| GFP | Microscopic | Descriptive | Terminus |
| immuno- | observations | statistics | |
| histochemistry | (subcontract) | | |

### Example 10. Efficacy Study and Long-Term PK Study of Intravitreal AAV Vector Candidates in a Non-Human Primate Model of Laser-Induced Choroidal Neovascularization

Determination of long-term ocular PK of expressed gene & evaluation of the efficacy of adeno-associated viral (AAV) vector candidates expressing Aflibercept, targeting wet age-related macular degeneration (wAMD), diabetic macular edema (DME), retinal vein occlusion (RVO) and polypoidal choroidal vasculopathy (PCV), following intravitreal (IVT) administration in African green monkeys in a laser-induced model of choroidal neovascularization (CNV).

Monkeys will undergo baseline screening to assess AAV neutralizing antibody (nAb) seronegativity, complete blood count (CBC), general well-being, and ocular health by tonometry, slit lamp biomicroscopy, fundoscopy, color fundus photography, fluorescein angiography (FA), confocal scanning laser ophthalmoscopy (cSLO), optical coherence tomography (OCT). nAb negative monkeys with normal findings will be enrolled in the study and assigned to treatment groups (Table 24) randomized by sex and baseline body weight. To accommodate the time necessary for imaging, monkeys will be divided into 3 cohorts for laser-induction of CNV, dosing, and imaging on successive days (Table 25). For baseline screening and all subsequent procedures, anesthesia will be achieved with intramuscular ketamine (8 mg/kg) and xylazine (1.6 mg/kg) to effect, and pupil dilation with topical 10% phenylephrine, 1% tropicamide and/or 1% cyclopentolate.

TABLE 24
Treatment assignment

| | | | | | Concentration | Volume | Dose | |
|---|---|---|---|---|---|---|---|---|
| Group | N | Eye | Treatment | Route | (vg/mL) | (μL/eye) | (vg/eye) | Test Article Required |
| 1 | [symbol] | OU | Null control | IVT | 4 × 10¹³ | 50 | 2 × 10¹² | (50 μL + 20 μL |
| | 9 | | AAV6.N54-Δ120 | | | | | overage) × 18 = 1260 μL |
| 2 | [symbol] | OU | AVMX-116 | IVT | 4 × 10¹³ | 50 | 2 × 10¹² | (50 μL + 20 μL |
| | 9 | | AA6.N54.120 | | | | | overage) × 18 = 1260 μL |
| 3 | 9 | OU | Aflibercept | IVT | 1 mg/25 μL | 30 | 1.2 mg | (30 μL + 20 μL |
| | | | | | | | | overage) × 12 = 600 μL |
| 4 | 6 | OU | AVMX-116 for | IVT | 4 × 10¹³ | 50 | 2 × 10¹² | (50 μL + 20 μL |
| | | | ocular PK | | | | | overage) × 18 = 1260 μL |
| 5 | 9 | OU | AVMX-110 | IVT | 4 × 10¹³ | 50 | 2 × 10¹² | (50 μL + 20 μL |
| | | | AAV2.N54.120 | | | | | overage) × 18 = 1260 μL |

TABLE 25
Study Schedule

| | | | | Study Interval | | | | | | | | | | | | | |
|---|---|---|---|---|---|---|---|---|---|---|---|---|---|---|---|---|---|
| | | | Wk | Wk | Wk | Wk | Wk | Wk | Wk | Wk | Wk | Wk | Wk | Wk | Wk | Wk | Wk |
| | | | −3 | −1 | 0 | 0 | 1 | 2 | 3 | 4 | 6 | 8 | 10 | 12 | 12 | 14 | 16 |
| | | | Day | Day | Day | Day | Day | Day | Day | Day | Day | Day | Day | Day | Day | Day | Day |
| Event | # | Eye | −21ª | −7 | −1 | 0*** | 7 | 14 | 21 | 28 | 42* | 56 | 70 | 84 | 86 | 100 | 114 |
| IVT vector dosing | 33 | OU | — | — | — | X | — | — | — | — | — | — | — | — | — | — | — |
| IVT Eylea | 9 | OU | — | — | — | — | — | — | — | — | X | — | — | — | — | — | — |
| dosing ** | | | | | | | | | | | | | | | | | |
| Methylprednisolone | 42 | — | — | — | X | — | X | X | X | X | — | — | — | — | — | — | — |
| Laser | 36 | OS | — | — | — | — | — | — | — | — | X | — | — | — | — | — | — |
| 2^{nd} Laser | 36 | OD | | | | | | | | | | | | | X | | |
| Body weight | 42 | — | X | X | — | X | X | X | X | X | X | X | — | X | X | — | X |
| Physical exam | 42 | — | X | — | — | — | — | — | — | — | — | — | — | X | — | — | X |
| Tonometry | 42 | OU | — | X | — | — | — | X | — | X | — | — | X | — | — | — | X |
| Slit lamp exam | 42 | OU | X | X | — | — | — | X | — | X | X | — | X | — | — | — | X (£) |
| Color fundus photos | 36 | OU | — | X | — | — | — | — | — | X | X | X | X | — | — | X | X |
| FA | 36 | OU | — | X | — | — | — | — | — | — | — | X | X | — | — | X | X |
| cSLO | 42 | OU | — | X | — | — | — | — | — | X | — | — | | X | — | — | X |
| | [symbol] | | | | | | | | | | | | | | | | |
| OCT | 42 | OU | — | X | — | — | — | — | — | X | — | — | | X | — | — | X |
| Serum | 42 | — | X | — | — | — | — | — | X | — | — | — | | X | — | — | X |
| CBC | 42 | — | X | — | — | — | — | — | — | — | — | — | | X | — | — | X |
| Aqueous tap | 36 | OU | — | — | — | X | — | — | X | — | — | — | — | — | — | — | X |
| Vitreous tap | −36 | OU | — | — | — | X | — | — | X | — | — | — | — | — | — | — | X |
| Eye collection | 36 | OU | — | — | — | — | — | — | — | — | — | — | | — | — | — | X |
| Group 4 AVMX- | 6 | OU | | | | X do | | | X do | | | | Cull | | | | Cull |
| 116 for ocular PK | | | | | | Aq | | | Aq | | | | n = 2$ | | | | n = 2$ |
| specific events | | | | | | & | | | & | | | | X | | | | X |
| | | | | | | Vit | | | Vit | | | | | | | | |
| | | | | | | tap | | | tap | | | | | | | | |

# 42 animals enrolled, >72? screened for neutralizing antibodies and up to >24-36? screened for ophthalmic health X indicates event occurring in all groups
*Color fundus photographs will be conducted immediately after laser. Topical 1% atropine gel will be applied one or two nights prior to laser treatment
**Group 6 will be dosed following laser model induction
***Day 0 aqueous and vitreous taps will be taken prior to IVT dosing
$PK culled animals: n = 2, d70, d114 & d169 - eye collection for aqueous & vitreous PK/ADA & full mount for retinal toxicity analysis, sera taken for PK & ADA, livers removed to IHC for tox
n = 2 d169 IOP & slit lamp prior to cull

### Systemic Immunosuppression Regimen

Intramuscular (IM) delivery of methylprednisone (8 mg/kg) on study day −1 and weekly for another 4 weeks (Table 25).

### Intravitreal Dosing

Topical proparacaine 0.5% will be administered, an eye speculum placed then the ocular surface rinsed with 5% Betadine solution followed by a sterile 0.9% saline rinse. IVT injections will be performed in both eyes (OU) according to the treatment assignment (Table 24) using a 31-gauge needle inserted inferotemporally at the level of the ora serrata mm posterior to the limbus. Following injection, a topical antibiotic ophthalmic ointment (neomycin, polymyxin, bacitracin or equivalent) will be administered.

### Laser Induction of CNV

Laser photocoagulation will be conducted at the designates time point (Table 25). Pupil dilation will be achieved prior to laser treatment with topical 10% phenylephrine hydrochloride and 1% cyclopentolate ophthalmic solutions. Topical administration of 1% atropine ophthalmic gel will be additionally applied one or two nights prior to the laser treatment to enhance mydriasis. Six laser spots will be symmetrically placed within the perimacular region in each eye by an ophthalmologist employing an Iridex Oculight TX 532 nm laser with a laser duration of 100 ms, spot size 50 μm, power 750 mW. Color fundus photography will be performed immediately after the laser treatment to document the laser lesions. Any spots demonstrating severe retinal/subretinal hemorrhage immediately post-laser and not resolving by the time of follow-up examinations will be excluded from analyses.

### Tonometry

At designated time points (Table 25) intraocular pressure (TOP) measurements will be performed using a TonoVet (iCare, Finland) tonometer set to the dog (d) calibration setting. Three measures will be taken from each eye at each time point and the mean TOP defined.

### Slit Lamp Biomicroscopy

Intraocular inflammation will be examined with slit lamp biomicroscopy at designated time points (Table 25). Scoring will be applied to qualitative clinical ophthalmic findings using a nonhuman primate ophthalmic exam scoring system with a summary score derived from exam components.

### Color Fundus Photography and Fluorescein Angiography

At designated time points (Table 25) bilateral color fundus images will be captured with 50° of view centered on the fovea using a Topcon TRC-50EX retinal camera with Canon 6D digital imaging hardware and New Vision Fundus Image Analysis System software. Fluorescein angiography (FA) will be performed with intravenous administration of 0.1 mL/kg of 10% sodium fluorescein. OD FA will precede OS angiography by greater than 2 hours to allow washout of the fluorescein between angiogram image series. Fluorescein leakage in angiograms of CNV lesions will be graded (I-IV; Table 26) assessing composites generated after uniform adjustment of image intensity. Image fluorescence densitometry analysis of late-stage raw angiograms will also be performed using ImageJ software.

TABLE 26
Laser Lesion Grading Scales

| Lesion | |
|---|---|
| Grade | Definition |
| I | No hyperfluorescence - Compare pre-FA with 30 sec post-FA. |
| | Look for absence of hyperfluorescence in lesion |
| II | Hyperfluorescence without leakage - Compare 30 sec FA with |
| | 3 and 6 minute FA. Look for hyperfluorescence without |
| | significant residual staining in 6 min FA. |
| III | Hyperfluorescence early or mid-transit and late leakage - |
| | Compare 30 sec FA with 3 and 6 minute FA. Look for |
| | significant residual staining in lesion at 6 min FA. |
| IV | Hyperfluorescence early or mid-transit and late leakage |
| | extending beyond the borders of the treated area - Compare 30 |
| | sec FA with 3 and 6 minute FA. Look for consistent staining |
| | beyond the border of the lesion as seen in 30 sec FA. |

### Optical Coherence Tomography

At designated time points (Table 25) OCT will be performed using a Heidelberg Spectralis OCT Plus with eye tracking and HEYEX image capture and analysis software. An overall volume scan of encompassing the posterior retina will be performed. At examinations prior to laser, the retinal thickness map and cross-sectional display image will be obtained. At post-laser examinations, six star-shaped scans per eye, centered on each lesion, will be performed, as well as an overall volume scan of the entire macula encompassing the six laser spots at a dense scan interval. The principal axis of maximal CNV complex formation within each star-shaped scan at each laser lesion will be defined and the CNV complex area measured using the freehand tool within ImageJ to delineate the CNV complex boundary and calculate maximum complex area in square microns (μm²).

### Clinical Observations

General wellbeing will be confirmed twice daily by cage side observations beginning one week prior to dosing. Daily individual food consumption will be assessed by visual inspection of the feed pan or cage floor prior to cage washing following routine feeding for overall appetite. During these observations the animals will be observed for signs of ocular inflammation, with special attention to such symptoms as squinting or eye rubbing.

### Body Weights

Body weights will be collected at designated time points (Table 25).

### Serum Collection

Whole blood (4 mL) will be collected via the femoral or saphenous vein at designated time points (Table 24) while animals are maintained under sedation as detailed previously. Blood will be transferred to vacutainer tubes (in the absence of anticoagulant) and incubated at room temperature for approximately 1 hour before centrifugation 4000 rpm for 10 min at 4° C. and isolation of serum aliquots (˜0.75 mL×2 aliquots per time point). Aliquots will be stored and shipped below −70° C. to a Sponsor designated laboratory for nAb analysis.

### Aqueous and Vitreous Humor Collection

Aqueous humor (5 μL each and full volume ˜150 μL at study termination) will be sampled at designated time points (Table 25) using a 0.3 mL insulin syringe with a 31-gauge needle after sterile preparation of the eye as for IVT injections. Vitreous humor (50 μL each and ˜150 μL at study termination) will be sampled at designated time points (Table 25) using a 0.5 mL insulin syringe with a 28-gauge needle after sterile preparation of the eye as for IVT injections. Following bilateral aqueous and/or vitreous taps (except at study terminus), a topical triple antibiotic neomycin, polymyxin, bacitracin ophthalmic ointment (or equivalent) will be administered. Aqueous and vitreous samples will be transferred into pre-labeled cryotubes and placed on wet ice and stored below −70° C. within 30 minutes of collection.

### Termination

Animals will be euthanized with sodium pentobarbital (100 mg/kg IV to effect) after confirming pre-terminus image quality.

### Eye Collection

Globes will be enucleated and excess orbital tissue will be trimmed. Eye collection for flat mount immunohistochemistry (IHC) staining and image analysis: Three pair of eyes from each group will be injected with ˜200 4% paraformaldehyde (PFA) then post-fixed in PFA for 6 hours and stored and shipped at 4° C. in a Credo Cube temperature-controlled cold container to a Sponsor designated laboratory for further processing. The anterior segment will be removed by a circumferential cut at the level of the ora serrata followed by longitudinal cuts in the eyecup to allow flat mounting for IHC and confocal imaging. Eye collection for protein analysis: Three pair of eyes from each group will be flash frozen in liquid nitrogen vapor prior to dissecting at room temperature along natural tissue planes. The anterior segment will be removed, and longitudinal cuts will then be placed in the frozen eye cup to flat mount the frozen posterior pole and collect the vitreous. Vitreous humor (˜2.5 mL full volume) will be divided into two aliquots. The retina will be separated from the RPE/choroid, and each collected individually.

### Data Analysis

Data generated from protocol-defined endpoints listed in Table 25 will be collated, summarized, and analyzed as detailed in Table 27. Specified statistical analyses will be performed where data meets required assumptions. If data fails to meet assumptions for defined statistical methods alternative methods may be employed where possible. P values ≤0.05 will be considered statistically significant.

TABLE 27
Data analysis categories

| Endpoint | Data Type | Statistics | Time points |
|---|---|---|---|
| Slit lamp | Graded clinical | Mixed model repeated | All defined in |
| | scoring | measures | Table 25 |
| IOP | Numerical | Mixed model repeated | All defined in |
| | measures | measures | Table 25 |
| Color fundus | Image appendix | Descriptive statistics | All defined in |
| photographs | | | Table 25 |
| Fluorescein | Image appendix | N/A | All defined in |
| angiography | | | Table 25 |
| CNV lesion | Graded leakage | Fisher's exact test | All defined in |
| grade | scoring | | Table 25 |
| Lesion | ImageJ-based | Mixed model repeated | All defined in |
| fluorescein | computerized | measures | Table 25 |
| intensity | fluorescein | | |
| | intensity | | |
| | analysis | | |
| CNV | Numerical | Mixed model repeated | All defined in |
| complex | measures | measures | Table 25 |
| size | (OCT) | | |
| Fibrosis | Graded scoring | Descriptive statistics | All defined in |
| histo- | (subcontract) | | Table 25 |
| pathology | | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-132. (canceled)

133. An AAV capsid comprising a polypeptide sequence of:
(SEQ ID NO: 94)
(a) L-A-L-G-X3-X1-X1-X4;
(SEQ ID NO: 95)
(b) L-K-L-G-X3-X1-X1-X4;
or
(SEQ ID NO: 96)
(c) V-K-L-G-X3-X1-X1-X4,
wherein:
X1 is Alanine (A), Asparagine (N), Glutamine (Q), Serine (S), Threonine (T), Glutamic Acid (E), Aspartic Acid (D), Lysine (K), Arginine (R), or Histidine (H);
X3 is E, S, or Q; and
X4 is K, R, E, or A, and
wherein the polypeptide sequence is in a VP domain of the AAV capsid.

134. The AAV capsid of claim 133, wherein the polypeptide sequence is (a), and wherein (a) is:
(SEQ ID NO: 99)
(i) L-A-L-G-X3-X1-T-X4;
or
(SEQ ID NO: 100)
ii) L-A-L-G-X3-X1-S-X4.

135. The AAV capsid of claim 134, wherein the polypeptide sequence is (i), and wherein (i) is:
(SEQ ID NO: 101)
(A) L-A-L-G-X3-X1-T-R;
(SEQ ID NO: 102)
(B) L-A-L-G-X3-X1-T-K;
(SEQ ID NO: 103)
(C) L-A-L-G-X3-X1-T-E;
or
(SEQ ID NO: 104)
(D) L-A-L-G-X3-X1-T-A.

136. The AAV capsid of claim 134, wherein the polypeptide sequence is (ii), and wherein (ii) is:
(SEQ ID NO: 105)
L-A-L-G-X3-X1-S-K

137. The AAV capsid of claim 133, wherein the polypeptide sequence is (b), and wherein (b) is
(SEQ ID NO: 106)
(i) L-K-L-G-X3-X1-T-X4

138. The AAV capsid of claim 137, wherein (i) is L-K-L-G-X3-X1-T-K (SEQ ID NO: 107).

139. The AAV capsid of claim 133, wherein the polypeptide sequence is (c), and wherein (c) is:
(SEQ ID NO: 97)
(i) V-K-L-G-X3-X1-T-X4;
or
(SEQ ID NO: 98)
(ii) V-K-L-G-X3-X1-X1-K.

140. The AAV capsid of claim 133, wherein the serotype of the AAV capsid is selected from the group consisting of: AAV1, AAV2, AAV3, AAV4, AAV5, AAV6, AAV7, AAV8, AAV9, AAV10, AAV11, AAV12, and AAV13.

141. The AAV capsid of claim 133, wherein the AAV capsid is a chimeric AAV capsid comprising an amino acid sequence from two or more serotypes selected from the group consisting of: AAV1, AAV2, AAV3, AAV4, AAV5, AAV6, AAV7, AAV8, AAV9, AAV10, AAV11, AAV12, and AAV13.

142. The AAV capsid of claim 133, wherein the AAV capsid is of serotype AAV2 or is a chimeric AAV capsid comprising an amino acid sequence from serotype AAV2.

143. The AAV capsid of claim 133, wherein the AAV capsid comprises an amino acid sequence having at least about 60% sequence identity to the amino acid sequence of any one of SEQ ID NOS: 28-47.

144. The AAV capsid of claim 133, wherein the polypeptide sequence is in the GH loop or loop IV of the AAV capsid.

145. The AAV capsid of claim 133, wherein the polypeptide sequence is an insertion within:
(i) amino acid positions 570-611 relative to AAV2 capsid;
(ii) amino acid positions 571-612 relative to AAV1 capsid;
(iii) amino acid positions 560-601 relative to AAV5 capsid;
(iv) amino acid positions 571-612 relative to AAV6 capsid;
(v) amino acid positions 572 to 613 relative to AAV7 capsid;
(vi) amino acid positions 573 to 614 relative to AAV8 capsid;
(vii) amino acid positions 571 to 612 relative to AAV9 capsid; or
(viii) amino acid positions 573 to 614 relative to AAV10 capsid.

146. The AAV capsid of claim 133, wherein the polypeptide sequence is an insertion at position 452, 453, 466, 467, 468, 471, 585, 586, 587, and/or 588 relative to SEQ ID NO: 1.

147. The AAV capsid of claim 133, wherein the AAV capsid further comprises at least one amino acid mutation relative to a wild-type AAV capsid.

148. The AAV capsid of claim 147, wherein the mutation is at amino acid position 452, 453, 466, 467, 468, 471, 585, 586, 587, and/or 588 relative to SEQ ID NO: 1.

149. The AAV capsid of claim 148, wherein the mutation is R to A at amino acid position 585 or 588 relative to SEQ ID NO: 1.

150. A vector comprising a polynucleotide sequence encoding the AAV capsid of claim 1.

151. The vector of claim 150, wherein the AAV capsid is of serotype AAV2 or is a chimeric AAV capsid comprising an amino acid sequence from serotype AAV2.

152. The vector of claim 150, wherein the vector comprises a polynucleotide sequence encoding a transgene.


SEQUENCE LISTING (133 sequences)

SEQ ID NO: 1 - protein, 735 aa, Adeno-associated dependoparvovirus A [sequence omitted]

SEQ ID NO: 2 - protein, 734 aa, Adeno-associated dependoparvovirus A
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 3 - protein, 724 aa, Adeno-associated dependoparvovirus B
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - protein, 736 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 733 aa, Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 6 - protein, 10 aa, synthetic construct
  REGION 1..10: note = Description of Artificial Sequence: Synthetic peptide
LALGETTRPA

SEQ ID NO: 7 - protein, 10 aa, synthetic construct
  REGION 1..10: note = Description of Artificial Sequence: Synthetic peptide
LALGETTKPA

SEQ ID NO: 8 - protein, 8 aa, synthetic construct
  REGION 1..8: note = Description of Artificial Sequence: Synthetic peptide
ALGETTKP

SEQ ID NO: 9 - protein, 8 aa, synthetic construct
  REGION 1..8: note = Description of Artificial Sequence: Synthetic peptide
ALGETTKP

SEQ ID NO: 10 - protein, 10 aa, synthetic construct
  REGION 1..10: note = Description of Artificial Sequence: Synthetic peptide
LALGETTKPA

SEQ ID NO: 11 - protein, 10 aa, synthetic construct
  REGION 1..10: note = Description of Artificial Sequence: Synthetic peptide
LKLGQTTKPK

SEQ ID NO: 12 - protein, 10 aa, synthetic construct
  REGION 1..10: note = Description of Artificial Sequence: Synthetic peptide
LALGQTTKPK

SEQ ID NO: 13 - protein, 10 aa, synthetic construct
  REGION 1..10: note = Description of Artificial Sequence: Synthetic peptide
LKLGQTTKPA

SEQ ID NO: 14 - protein, 10 aa, synthetic construct
  REGION 1..10: note = Description of Artificial Sequence: Synthetic peptide
LALGQTTKPA

SEQ ID NO: 15 - protein, 10 aa, synthetic construct
  REGION 1..10: note = Description of Artificial Sequence: Synthetic peptide
LALGQTTKPA

SEQ ID NO: 16 - protein, 10 aa, synthetic construct
  REGION 1..10: note = Description of Artificial Sequence: Synthetic peptide
LALGQTTEPA

SEQ ID NO: 17 - protein, 10 aa, synthetic construct
  REGION 1..10: note = Description of Artificial Sequence: Synthetic peptide
LALGQTTKPA

SEQ ID NO: 18 - protein, 10 aa, synthetic construct
  REGION 1..10: note = Description of Artificial Sequence: Synthetic peptide
LALGQTTKPA

SEQ ID NO: 19 - protein, 10 aa, synthetic construct
  REGION 1..10: note = Description of Artificial Sequence: Synthetic peptide
VALGQTTKPA

SEQ ID NO: 20 - protein, 10 aa, synthetic construct
  REGION 1..10: note = Description of Artificial Sequence: Synthetic peptide
LALGESTARG

SEQ ID NO: 21 - protein, 10 aa, synthetic construct
  REGION 1..10: note = Description of Artificial Sequence: Synthetic peptide
LALGETSKRA

SEQ ID NO: 22 - protein, 10 aa, synthetic construct
  REGION 1..10: note = Description of Artificial Sequence: Synthetic peptide
LALGQSTKPA

SEQ ID NO: 23 - protein, 10 aa, synthetic construct
  REGION 1..10: note = Description of Artificial Sequence: Synthetic peptide
LALGQTTKPA

SEQ ID NO: 24 - protein, 10 aa, synthetic construct
  REGION 1..10: note = Description of Artificial Sequence: Synthetic peptide
LALGQTTKPA

SEQ ID NO: 25 - protein, 10 aa, synthetic construct
  REGION 1..10: note = Description of Artificial Sequence: Synthetic peptide
LALGQTTKPA

SEQ ID NO: 26 - protein, 20 aa, synthetic construct
  REGION 1..20: note = Description of Artificial Sequence: Synthetic peptide
LALGQTTKPALALGQTTKPA

SEQ ID NO: 27 - protein, 10 aa, synthetic construct
  REGION 1..10: note = Description of Artificial Sequence: Synthetic peptide
VKLGQTTKPA

SEQ ID NO: 28 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGETTRPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 30 - protein, 743 aa, synthetic construct
  REGION 1..743: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 31 - protein, 753 aa, synthetic construct
  REGION 1..753: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 32 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 33 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 34 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 36 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 37 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTEPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRLALGQTTKPAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 39 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRLALGQTTKPAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 40 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 41 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGESTARGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 42 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGETSKRANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 43 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGQSTKPANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 44 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSLALGQTTKPAGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 45 - protein, 755 aa, synthetic construct
  REGION 1..755: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 46 - protein, 755 aa, synthetic construct
  REGION 1..755: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPALALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 47 - protein, 745 aa, synthetic construct
  REGION 1..745: note = Description of Artificial Sequence: Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVAKGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 48 - DNA, 25 bp, synthetic construct
  misc_feature 1..25: note = Description of Artificial Sequence: Synthetic primer
  source 1..25: mol_type = other DNA
cgagactgcaggctctagattacag

SEQ ID NO: 49 - DNA, 25 bp, synthetic construct
  misc_feature 1..25: note = Description of Artificial Sequence: Synthetic primer
  source 1..25: mol_type = other DNA
ggagtaccagctcccgtacgtcctc

SEQ ID NO: 50 - DNA, 49 bp, synthetic construct
  misc_feature 1..49: note = Description of Artificial Sequence: Synthetic primer
  source 1..49: mol_type = other DNA
ctagcactcggcgaaacaacaagacctgctaccaccacgcagtcaaggc

SEQ ID NO: 51 - DNA, 49 bp, synthetic construct
  misc_feature 1..49: note = Description of Artificial Sequence: Synthetic primer
  source 1..49: mol_type = other DNA
agcaggtcttgttgtttcgccgagtgctagtccacttggagtgtttgtt

SEQ ID NO: 52 - DNA, 49 bp, synthetic construct
  misc_feature 1..49: note = Description of Artificial Sequence: Synthetic primer
  source 1..49: mol_type = other DNA
ctagcactcggcgaaacaacaaagcctgctaggcaagcagctaccgcag

SEQ ID NO: 53 - DNA, 49 bp, synthetic construct
  misc_feature 1..49: note = Description of Artificial Sequence: Synthetic primer
  source 1..49: mol_type = other DNA
agcaggctttgttgtttcgccgagtgctaggttgcctctctggaggttg

SEQ ID NO: 54 - DNA, 44 bp, synthetic construct
  misc_feature 1..44: note = Description of Artificial Sequence: Synthetic primer
  source 1..44: mol_type = other DNA
aggctttgttgtttcgccgagtgcacttggagtgtttgttctgc

SEQ ID NO: 55 - DNA, 44 bp, synthetic construct
  misc_feature 1..44: note = Description of Artificial Sequence: Synthetic primer
  source 1..44: mol_type = other DNA
gcactcggcgaaacaacaaagcctggaaccaccacgcagtcaag

SEQ ID NO: 56 - DNA, 25 bp, synthetic construct
  misc_feature 1..25: note = Description of Artificial Sequence: Synthetic primer
  source 1..25: mol_type = other DNA
ggagtaccagctcccgtacgtcctc

SEQ ID NO: 57 - DNA, 49 bp, synthetic construct
  misc_feature 1..49: note = Description of Artificial Sequence: Synthetic primer
  source 1..49: mol_type = other DNA
cttaggctttgttgtctggccgagctttaggttgcctctctggaggttg

SEQ ID NO: 58 - DNA, 49 bp, synthetic construct
  misc_feature 1..49: note = Description of Artificial Sequence: Synthetic primer
  source 1..49: mol_type = other DNA
ctaaagctcggccagacaacaaagcctaagaggcaagcagctaccgcag

SEQ ID NO: 59 - DNA, 25 bp, synthetic construct
  misc_feature 1..25: note = Description of Artificial Sequence: Synthetic primer
  source 1..25: mol_type = other DNA
cgagactgcaggctctagattacag

SEQ ID NO: 60 - DNA, 49 bp, synthetic construct
  misc_feature 1..49: note = Description of Artificial Sequence: Synthetic primer
  source 1..49: mol_type = other DNA
cttaggctttgttgtctggccgagtgctaggttgcctctctggaggttg

SEQ ID NO: 61 - DNA, 49 bp, synthetic construct
  misc_feature 1..49: note = Description of Artificial Sequence: Synthetic primer
  source 1..49: mol_type = other DNA
ctagcactcggccagacaacaaagcctaagaggcaagcagctaccgcag

SEQ ID NO: 62 - DNA, 49 bp, synthetic construct
  misc_feature 1..49: note = Description of Artificial Sequence: Synthetic primer
  source 1..49: mol_type = other DNA
tgcaggctttgttgtctggccgagctttaggttgcctctctggaggttg

SEQ ID NO: 63 - DNA, 49 bp, synthetic construct
  misc_feature 1..49: note = Description of Artificial Sequence: Synthetic primer
  source 1..49: mol_type = other DNA
ctaaagctcggccagacaacaaagcctgcaaggcaagcagctaccgcag

SEQ ID NO: 64 - DNA, 49 bp, synthetic construct
  misc_feature 1..49: note = Description of Artificial Sequence: Synthetic primer
  source 1..49: mol_type = other DNA
tgcaggctttgttgtctggccgagtgctaggttgcctctctggaggttg

SEQ ID NO: 65 - DNA, 49 bp, synthetic construct
  misc_feature 1..49: note = Description of Artificial Sequence: Synthetic primer
  source 1..49: mol_type = other DNA
ctagcactcggccagacaacaaagcctgcaaggcaagcagctaccgcag

SEQ ID NO: 66 - DNA, 45 bp, synthetic construct
  misc_feature 1..45: note = Description of Artificial Sequence: Synthetic primer
  source 1..45: mol_type = other DNA
tagctgcttgtgcgttgcctgcctggaggttggtagatacagaac

SEQ ID NO: 67 - DNA, 45 bp, synthetic construct
  misc_feature 1..45: note = Description of Artificial Sequence: Synthetic primer
  source 1..45: mol_type = other DNA
acctccaggcaggcaacgcacaagcagctaccgcagatgtcaaca

SEQ ID NO: 68 - DNA, 45 bp, synthetic construct
  misc_feature 1..45: note = Description of Artificial Sequence: Synthetic primer
  source 1..45: mol_type = other DNA
tagctgcttgcctgttgcctgcctggaggttggtagatacagaac

SEQ ID NO: 69 - DNA, 45 bp, synthetic construct
  misc_feature 1..45: note = Description of Artificial Sequence: Synthetic primer
  source 1..45: mol_type = other DNA
acctccaggcaggcaacaggcaagcagctaccgcagatgtcaaca

SEQ ID NO: 70 - DNA, 45 bp, synthetic construct
  misc_feature 1..45: note = Description of Artificial Sequence: Synthetic primer
  source 1..45: mol_type = other DNA
tagctgcttgtgcgttgcctctctggaggttggtagatacagaac

SEQ ID NO: 71 - DNA, 45 bp, synthetic construct
  misc_feature 1..45: note = Description of Artificial Sequence: Synthetic primer
  source 1..45: mol_type = other DNA
acctccagagaggcaacgcacaagcagctaccgcagatgtcaaca

SEQ ID NO: 72 - DNA, 49 bp, synthetic construct
  misc_feature 1..49: note = Description of Artificial Sequence: Synthetic primer
  source 1..49: mol_type = other DNA
ctagcactcggccagacaacaaagcctgcaaccaccacgcagtcaaggc

SEQ ID NO: 73 - DNA, 49 bp, synthetic construct
  misc_feature 1..49: note = Description of Artificial Sequence: Synthetic primer
  source 1..49: mol_type = other DNA
tgcaggctttgttgtctggccgagtgctagtccacttggagtgtttgtt

SEQ ID NO: 74 - DNA, 49 bp, synthetic construct
  misc_feature 1..49: note = Description of Artificial Sequence: Synthetic primer
  source 1..49: mol_type = other DNA
ctagcactcggccagacaacaaagcctgcaggaaccaccacgcagtcaa

SEQ ID NO: 75 - DNA, 49 bp, synthetic construct
  misc_feature 1..49: note = Description of Artificial Sequence: Synthetic primer
  source 1..49: mol_type = other DNA
tgcaggctttgttgtctggccgagtgctagacttggagtgtttgttctg

SEQ ID NO: 76 - DNA, 49 bp, synthetic construct
  misc_feature 1..49: note = Description of Artificial Sequence: Synthetic primer
  source 1..49: mol_type = other DNA
ctagcactcggccagacaacagagcctgcaaggcaagcagctaccgcag

SEQ ID NO: 77 - DNA, 49 bp, synthetic construct
  misc_feature 1..49: note = Description of Artificial Sequence: Synthetic primer
  source 1..49: mol_type = other DNA
tgcaggctctgttgtctggccgagtgctaggttgcctctctggaggttg

SEQ ID NO: 78 - DNA, 49 bp, synthetic construct
  misc_feature 1..49: note = Description of Artificial Sequence: Synthetic primer
  source 1..49: mol_type = other DNA
ctagcactcggccagacaacaaagcctgcacaagcagctaccgcagatg

SEQ ID NO: 79 - DNA, 49 bp, synthetic construct
  misc_feature 1..49: note = Description of Artificial Sequence: Synthetic primer
  source 1..49: mol_type = other DNA
tgcaggctttgttgtctggccgagtgctagcctgttgcctctctggagg

SEQ ID NO: 80 - DNA, 49 bp, synthetic construct
  misc_feature 1..49: note = Description of Artificial Sequence: Synthetic primer
  source 1..49: mol_type = other DNA
ctagcactcggccagacaacaaagcctgcaggcaacaggcaagcagcta

SEQ ID NO: 81 - DNA, 49 bp, synthetic construct
  misc_feature 1..49: note = Description of Artificial Sequence: Synthetic primer
  source 1..49: mol_type = other DNA
tgcaggctttgttgtctggccgagtgctagtctctggaggttggtagat

SEQ ID NO: 82 - DNA, 49 bp, synthetic construct
  misc_feature 1..49: note = Description of Artificial Sequence: Synthetic primer
  source 1..49: mol_type = other DNA
gtggcactcggccagacaacaaagcctgcaaggcaagcagctaccgcag

SEQ ID NO: 83 - DNA, 49 bp, synthetic construct
  misc_feature 1..49: note = Description of Artificial Sequence: Synthetic primer
  source 1..49: mol_type = other DNA
tgcaggctttgttgtctggccgagtgccacgttgcctctctggaggttg

SEQ ID NO: 84 - DNA, 49 bp, synthetic construct; identical to SEQ ID NO: 74
  misc_feature 1..49: note = Description of Artificial Sequence: Synthetic primer
  source 1..49: mol_type = other DNA

SEQ ID NO: 85 - DNA, 49 bp, synthetic construct; identical to SEQ ID NO: 75
  misc_feature 1..49: note = Description of Artificial Sequence: Synthetic primer
  source 1..49: mol_type = other DNA

SEQ ID NO: 86 - DNA, 49 bp, synthetic construct
  misc_feature 1..49: note = Description of Artificial Sequence: Synthetic primer
  source 1..49: mol_type = other DNA
ctggcactcggcgagtcgacagcgcggggcaacaggcaagcagctaccg

SEQ ID NO: 87 - DNA, 49 bp, synthetic construct
  misc_feature 1..49: note = Description of Artificial Sequence: Synthetic primer
  source 1..49: mol_type = other DNA
gccccgcgctgtcgactcgccgagtgccaggcctctctggaggttggta

SEQ ID NO: 88 - DNA, 49 bp, synthetic construct
  misc_feature 1..49: note = Description of Artificial Sequence: Synthetic primer
  source 1..49: mol_type = other DNA
ctggcactcggcgagacaagcaagcgggcaaacaggcaagcagctaccg

SEQ ID NO: 89 - DNA, 49 bp, synthetic construct
  misc_feature 1..49: note = Description of Artificial Sequence: Synthetic primer
  source 1..49: mol_type = other DNA
tgcccgcttgcttgtctcgccgagtgccaggcctctctggaggttggta

SEQ ID NO: 90 - DNA, 49 bp, synthetic construct
  misc_feature 1..49: note = Description of Artificial Sequence: Synthetic primer
  source 1..49: mol_type = other DNA
ctagcactcggccagagcacaaagcctgcaaacaggcaagcagctaccg

SEQ ID NO: 91 - DNA, 49 bp, synthetic construct
  misc_feature 1..49: note = Description of Artificial Sequence: Synthetic primer
  source 1..49: mol_type = other DNA
tgcaggctttgtgctctggccgagtgctaggcctctctggaggttggta

SEQ ID NO: 92 - protein, 742 aa, Adeno-associated dependoparvovirus A
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 93 - skipped

SEQ ID NO: 94 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 7: note = A, N, Q, S, T, E, D, K, R, or H
  MOD_RES 8: note = K, R, E, or A
LALGXXXX

SEQ ID NO: 95 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 7: note = A, N, Q, S, T, E, D, K, R, or H
  MOD_RES 8: note = K, R, E, or A
LKLGXXXX

SEQ ID NO: 96 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 7: note = A, N, Q, S, T, E, D, K, R, or H
  MOD_RES 8: note = K, R, E, or A
VKLGXXXX

SEQ ID NO: 97 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 8: note = K, R, E, or A
VKLGXXTX

SEQ ID NO: 98 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 7: note = A, N, Q, S, T, E, D, K, R, or H
VKLGXXXK

SEQ ID NO: 99 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 8: note = K, R, E, or A
LALGXXTX

SEQ ID NO: 100 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 8: note = K, R, E, or A
LALGXXSX

SEQ ID NO: 101 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
LALGXXTR

SEQ ID NO: 102 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
LALGXXTK

SEQ ID NO: 103 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
LALGXXTE

SEQ ID NO: 104 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
LALGXXTA

SEQ ID NO: 105 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
LALGXXSK

SEQ ID NO: 106 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
  MOD_RES 8: note = K, R, E, or A
LKLGXXTX

SEQ ID NO: 107 - protein, 8 aa, synthetic construct
  REGION 1..8: note = Description of Artificial Sequence: Synthetic peptide
  MOD_RES 5: note = E, S, or Q
  MOD_RES 6: note = A, N, Q, S, T, E, D, K, R, or H
LKLGXXTK

SEQ ID NO: 108 - skipped

SEQ ID NO: 109 - RNA, 27 nt, synthetic construct
  misc_feature 1..27: note = Description of Artificial Sequence: Synthetic oligonucleotide
  source 1..27: mol_type = other RNA
cgcaatcagtgaatgcttatacatccg

SEQ ID NO: 110 - protein, 18 aa, synthetic construct
  REGION 1..18: note = Description of Artificial Sequence: Synthetic peptide
TPSGLALGETTRPATTTQ

SEQ ID NO: 111 - protein, 18 aa, synthetic construct
  REGION 1..18: note = Description of Artificial Sequence: Synthetic peptide
QRGNLALGETTKPARQAA

SEQ ID NO: 112 - protein, 16 aa, synthetic construct
  REGION 1..16: note = Description of Artificial Sequence: Synthetic peptide
NTPSALGETTKPGTTT

SEQ ID NO: 113 - protein, 16 aa, synthetic construct
  REGION 1..16: note = Description of Artificial Sequence: Synthetic peptide
NTPSALGETTKPGTTT

SEQ ID NO: 114 - protein, 18 aa, synthetic construct
  REGION 1..18: note = Description of Artificial Sequence: Synthetic peptide
QRGNLKLGQTTKPKRQAA

SEQ ID NO: 115 - protein, 18 aa, synthetic construct
  REGION 1..18: note = Description of Artificial Sequence: Synthetic peptide
QRGNLALGQTTKPKRQAA

SEQ ID NO: 116 - protein, 18 aa, synthetic construct
  REGION 1..18: note = Description of Artificial Sequence: Synthetic peptide
QRGNLKLGQTTKPARQAA

SEQ ID NO: 117 - protein, 18 aa, synthetic construct
  REGION 1..18: note = Description of Artificial Sequence: Synthetic peptide
QRGNLALGQTTKPARQAA

SEQ ID NO: 118 - protein, 18 aa, synthetic construct
  REGION 1..18: note = Description of Artificial Sequence: Synthetic peptide
TPSGLALGQTTKPATTTQ

SEQ ID NO: 119 - protein, 18 aa, synthetic construct
  REGION 1..18: note = Description of Artificial Sequence: Synthetic peptide
QRGNLALGQTTEPARQAA

SEQ ID NO: 120 - protein, 18 aa, synthetic construct
  REGION 1..18: note = Description of Artificial Sequence: Synthetic peptide
RGNRLALGQTTKPAQAAT

SEQ ID NO: 121 - protein, 18 aa, synthetic construct
  REGION 1..18: note = Description of Artificial Sequence: Synthetic peptide
NLQRLALGQTTKPAGNRQ

SEQ ID NO: 122 - protein, 18 aa, synthetic construct
  REGION 1..18: note = Description of Artificial Sequence: Synthetic peptide
QRGNVALGQTTKPARQAA

SEQ ID NO: 123 - protein, 18 aa, synthetic construct
  REGION 1..18: note = Description of Artificial Sequence: Synthetic peptide
LQRGLALGESTARGNRQA

SEQ ID NO: 124 - protein, 18 aa, synthetic construct
  REGION 1..18: note = Description of Artificial Sequence: Synthetic peptide
LQRGLALGETSKRANRQA

SEQ ID NO: 125 - protein, 18 aa, synthetic construct
  REGION 1..18: note = Description of Artificial Sequence: Synthetic peptide
LQRGLALGQSTKPANRQA

SEQ ID NO: 126 - protein, 18 aa, synthetic construct
  REGION 1..18: note = Description of Artificial Sequence: Synthetic peptide
NTPSLALGQTTKPAGTTT

SEQ ID NO: 127 - protein, 28 aa, synthetic construct
  REGION 1..28: note = Description of Artificial Sequence: Synthetic peptide
QRGNLALGQTTKPALALGQTTKPARQAA

SEQ ID NO: 128 - protein, 18 aa, synthetic construct
  REGION 1..18: note = Description of Artificial Sequence: Synthetic peptide
QRGNVKLGQTTKPARQAA

SEQ ID NO: 129 - protein, 11 aa, synthetic construct
  REGION 1..11: note = Description of Artificial Sequence: Synthetic peptide
LALGETSRPAG

SEQ ID NO: 130 - protein, 11 aa, synthetic construct
  REGION 1..11: note = Description of Artificial Sequence: Synthetic peptide
LKLGQTSKPAG

SEQ ID NO: 131 - protein, 11 aa, synthetic construct
  REGION 1..11: note = Description of Artificial Sequence: Synthetic peptide
LALGQTSKPAG

SEQ ID NO: 132 - protein, 11 aa, synthetic construct
  REGION 1..11: note = Description of Artificial Sequence: Synthetic peptide
LALGETSKPAG

SEQ ID NO: 133 - protein, 8 aa, synthetic construct
  REGION 1..8: note = Description of Artificial Sequence: Synthetic peptide
QRGNRQAA